(19) United States
(12) Patent Application Publication
(10) Pub. No.: US 2011/0142867 A1
(43) Pub. Date: Jun. 16, 2011

(54) IMMUNOTHERAPEUTIC METHODS AND SYSTEMS

(76) Inventors: Mark Larche, (US); Philip William Ledger, (US)

(21) Appl. No.: 12/860,216

(22) Filed: Aug. 20, 2010

Related U.S. Application Data
(63) Continuation of application No. 10/498,026, filed on Jun. 27, 2005, now abandoned.
(60) Provisional application No. 60/338,385, filed on Dec. 5, 2001.

(51) Int. Cl. A61K 39/00; A61K 39/35; A61P 19/02; A61P 25/28; A61P 3/10; A61P 27/02; A61P 11/00; A61P 1/00

(52) U.S. Cl. 424/185.1; 424/184.1; 424/275.1; 424/274.1

(57) ABSTRACT

Methods for desensitising an individual to a selected polypeptide antigen are provided. The methods entail administration of T cell epitope containing peptides from a polypeptide antigen in such a way as to establish a tolergeneic environment, that is, a state of hyporesponsiveness to the peptides. The selected polypeptide antigen is then administered such that the state of hyporesponsiveness and co-administration of the selected antigen are sufficient to desensitise the individual to the polypeptide antigen. Also provided are therapeutic systems useful in the methods of the invention, and the use of polypeptide antigens and peptides in the manufacture of medicaments in the methods of the invention.

### CROSS-REFERENCE TO RELATED APPLICATIONS

This application is a continuation of U.S. Ser. No. 10/498,026, filed Jun. 27, 2005, which is a §371 filing of PCT/GB2002/05548, filed Dec. 5, 2002, which claims the benefit of U.S. Ser. No. 60/338,385, filed Dec. 5, 2001, from which applications priority is claimed pursuant to the provisions of 35 U.S.C. §119 and §120, which applications are incorporated herein by reference in their entireties.

### FIELD OF THE INVENTION

The present invention relates to immunotherapeutic methods and systems and, in particular, it relates to methods of desensitising an individual to polypeptide antigens, particularly to polypeptide allergens.

### BACKGROUND OF THE INVENTION

The ability of the immune system to elicit a response to a particular molecule depends critically upon its ability to recognise the presence of an antigen. Classically, the term antigen has been associated with the ability of a molecule to be an antibody generator via induction of B-cells. It is now known, however, that T cells also possess the ability to recognise antigens. T-cell antigen recognition requires antigen presenting cells (APCs) to present antigen fragments (peptides) on their cell surface in association with molecules of the major histocompatibility complex (MHC). T cells use their antigen specific T-cell receptors (TCRs) to recognise the antigen fragments presented by the APC. Such recognition acts as a trigger to the immune system to generate a range of responses to eradicate the antigen which has been recognised.

T lymphocytes have been implicated in the pathogenesis of a wide variety of diseases involving immune recognition of antigens derived both from the internal (host) and external environments. Autoimmune diseases such as autoimmune thyroiditis, rheumatoid arthritis and lupus erythrematosus arise from the recognition by the immune system of host, or self, antigens.

Recognition of external antigens by the immune system of an organism, such as man, can in some cases result in diseases, known as atopic conditions. An example of the latter are the allergic diseases including asthma, atopic dermatitis and allergic rhinitis. In this group of diseases, B lymphocytes generate antibodies of the IgE class (in humans) which bind externally derived antigens, which are referred to in this context as allergens, since these molecules elicit an allergic response. Production of allergen-specific IgE is dependent upon T lymphocytes which are also activated by (are specific for) the allergen. Allergen-specific IgE antibodies bind to the surface of cells such as basophils and mast cells by virtue of the expression by these cells of surface receptors for IgE. Crosslinking of surface bound. IgE molecules by allergen results in degranulation of these effector cells causing release of inflammatory mediators such as histamine, 5-hydroxytryptamine and lipid mediators such as the sulphidoleukotrienes. In addition to IgE-dependent events, certain allergic diseases such as asthma are characterised by IgE-independent events. It has been demonstrated that the induction of the late phase reaction is at least in part, an IgE-independent event which is dependent upon the activation of allergen-specific T lymphocytes.

Allergic IgE-mediated diseases are currently normally treated with agents which provide symptomatic relief, or by prevention. Examples of such agents are anti-histamines, β₂ agonists, and glucocorticosteroids. In addition, some IgE-mediated diseases are treated by desensitisation procedures that involve the periodic injection of allergen components or extracts. Desensitisation treatments may induce an IgG response that competes with IgE for allergen, or they may induce specific suppressor T cells that block the synthesis of IgE directed against allergen. This form of treatment is not always effective and popes the risk of provoking serious side effects, particularly general anaphylactic shock. This can be fatal unless recognised immediately and treated with adrenaline.

### SUMMARY OF THE INVENTION

It is a primary object of the invention to provide a method for desensitising an individual to a selected polypeptide antigen. The method comprises: (a) administering a primary composition to the individual, wherein the primary composition comprises a first polypeptide antigen containing a T cell epitope, the individual has been previously exposed to the first polypeptide antigen, and administration of the primary composition is carried out in a manner sufficient to generate a hyporesponsive state against the first polypeptide antigen; and (b) administering a secondary composition to the individual, wherein the secondary composition comprises the selected polypeptide antigen and is coadministered with either the first polypeptide antigen or a larger molecule containing the first polypeptide antigen. The hyporesponsive state generated in step (a) and coadministration of the selected polypeptide antigen with the first polypeptide antigen or a larger molecule containing the first polypeptide antigen in step (b) are sufficient to desensitise the individual to the selected polypeptide antigen.

In certain aspects, the individual is allergic to the first polypeptide antigen. In other aspects, the first polypeptide antigen is an aero allergen. In a particularly preferred embodiment, the primary composition comprises one or more peptides the primary composition comprises one or more Fel d 1 peptides selected from the group consisting of EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOs 1-12.

The selected polypeptide antigen can be an allergen obtained or derived from any one or more of the following sources; latex; plants such as grass, tree, and ragweed; pollens; fungi and moulds; foods; stinging insects including wasps; the chirnomidae (non-biting midges); spiders and mites; flies such as housefly, fruit fly, sheep blow fly and screw worm fly; grain weevil; silkworm; honeybee; non-biting midge larvae; bee moth larvae; mealworm; cockroach; larvae of Tenibrio molitor beetle; and mammals such as cat, dog, horse, cow, pig, sheep, rabbit, rat, guinea pig, mice and gerbil.

In some embodiments, the selected polypeptide antigen is an allergen is selected from the group consisting of Der p 1; Der p 2; Der p 3; Der p 4; Der p 5; Der p 6; Der p 7; Der p 9; Der f 1; Der f 2; Der f 3; Der f 4; Der f 7; Fel d 1 chain 1 or 2; Hev b 1; Hev b 3; Lol p 1; Lol p 2a; Lol p 3; Lol p 5a; Lol p 5b; Lol p isoform 9; Lol p 11; Ole e 1; Par j P2; Par j P5; Par j P8; Par j P9; Par j 1; Phl p 1; Phl p 2; Phl p 5; Phl p 5b; Phl p 5a; VES V 5; VES M 1; VES V 1; VES V 2; VES VI; Bet v 1; Bet v 2; Bet v 3; Bet v 4; Que a I; Car b I; Aln g I; Rubisco; Ara h 1; Amb a 1; Amb a 2; Amb a 1.3; Amb a 1.2; Amb a 1.1; Cry j IB precursor; Cry j IA precursor; Cry j II precursor; Cry j II protein; Cry j I precursor; Can f 1; Can f 2; serum albumin fragment; Equ c1; Equ c 2; Eur m 1; POA P 9; Cr p1; Cr p2; Bla g 2; Bla g 4; and Bla g 5.

In other embodiments, the selected polypeptide antigen is obtained or derived from an autoantigen. In certain embodiments, the autoantigen is associated with any one of the following autoimmune disorders: Multiple Sclerosis; Diabetes; Rheumatoid Arthritis; Thyroiditis; Systemic Lupus Erythromatosus; Behcet's Disease; Coeliac Disease; or Myasthenia gravis. In particular preferred embodiments, the autoantigen is selected from the group consisting of: myelin basic protein (MBP); proteolipid protein (PLP); myelin oligodendrocyte glycoprotein (MOG); glutamic acid decarboxylase (GAD); insulin; IA-2 (a protein phosphatase-like molecule); collagen; heat shock proteins (HSP's); thyroglobulin; histone proteins; immunoglobulin heavy chain; S antigen from the eye (Sag); HLA-B44; HLA B51; HSP65; gliadin; and acetyl choline receptor. In still further embodiments, the selected polypeptide antigen is obtained or derived from a transplant antigen.

The compositions administered in the practice of the methods of the invention can be administered in a slow release formulation or in an oil and water emulsion. In addition, the compositions can be administered using intradermal injection, subcutaneous injection, intramuscular injection, intravenous injection, transdermal, intranasal, oral, intraocular, or intrathecal administration technique. In a preferred embodiment, one or more of the compositions is provided in a dry, powdered form and is administered using a transdermal particle injection technique.

It is also a primary object of the invention to provide a method for desensitising an individual to a selected polypeptide antigen. The method comprises: (a) administering a primary composition to the individual, wherein the primary composition comprises a peptide antigen obtained or derived from the selected polypeptide antigen and the peptide antigen contains a T cell epitope, the individual has been previously exposed to the peptide antigen, and administration of the primary composition is carried out in a manner sufficient to generate a hyporesponsive state against the peptide antigen; and (b) administering a secondary composition to the individual, wherein the secondary composition comprises the selected polypeptide antigen, whereby the hyporesponsive state generated in step (a) and administration of the secondary composition are sufficient to desensitise the individual to the selected polypeptide antigen.

In certain aspects, the individual is allergic to the first polypeptide antigen. In other aspects, the first polypeptide antigen is an aero allergen. In a particularly preferred embodiment, the primary composition comprises one or more peptides the primary composition comprises one or more Fel d 1 peptides selected from the group consisting of EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOs 1-12.

The selected polypeptide antigen can be an allergen obtained or derived from any one or more of the following sources: latex; plants such as grass, tree, and ragweed; pollens; fungi and moulds; foods; stinging insects including wasps; the chirnomidae (non-biting midges); spiders and mites; flies such as housefly, fruit fly, sheep blow fly and screw worm fly; grain weevil; silkworm; honeybee; non-biting midge larvae; bee moth larvae; mealworm; cockroach; larvae of Tenibrio molitor beetle; and mammals such as cat, dog, horse, cow, pig, sheep, rabbit, rat, guinea pig, mice and gerbil.

In some embodiments, the selected polypeptide antigen is an allergen is selected from the group consisting of Der p 1; Der p 2; Der p 3; Der p 4; Der p 5; Der p 6; Der p 7; Der p 9; Der f 1; Der f 2; Der f 3; Der f 4; Der f 7; Fel d 1 chain 1 or 2; Hev b 1; Hev b 3; Lol p 1; Lol p 2a; Lol p 3; Lol p 5a; Lol p 5b; Lol p isoform 9; Lol p 11; Ole e 1; Par j P2; Par j P5; Par j P8; Par j P9; Par j 1; Phl p 1; Phl p 2; Phl p 5; Phl p 5b; Phl p 5a; VES V 5; VES M 1; VES V 1; VES V 2; VES VI; Bet v 1; Bet v 2; Bet v 3; Bet v 4; Que a I; Car b I; Aln g I; Rubisco; Ara h 1; Amb a 1; Amb a 2; Amb a 1.3; Amb a 1.2; Amb a 1.1; Cry j IB precursor; Cry j IA precursor; Cry j II precursor; Cry j II protein; Cry j I precursor; Can f 1; Can f 2; serum albumin fragment; Equ c1; Equ c 2; Eur m 1; POA P 9; Cr p1; Cr p2; Bla g 2; Bla g 4; and Bla g 5.

The compositions administered in the practice of the methods of the invention can be administered in a slow release formulation or in an oil and water emulsion. In addition, the compositions can be administered using intradermal injection, subcutaneous injection, intramuscular injection, intravenous injection, transdermal, intranasal, oral, intraocular, or intrathecal administration technique. In a preferred embodiment, one or more of the compositions is provided in a dry, powdered form and is administered using a transdermal particle injection technique.

It is a still further primary object of the invention to provide therapeutic systems comprising compositions useful in the practice of the methods of the invention. It is also a primary object of the invention to provide for the use of a polypeptide antigen, peptide antigen, or whole antigen in the manufacture of a medicament suitable for use in the practice of the methods of the instant invention.

It is an advantage of the present invention that individuals who are multiply allergic to different allergens can be treated in a single, time-saving method. It is also an advantage of the present invention that a mild antigen (e.g., an aero allergen) can be used to establish a tolergeneic environment that then enables a more severe antigen (e.g., a nut allergen) to be administered and take advantage of the environment in a desensitisation to that more severe antigen.

These and other objects, aspects, embodiments and advantages of the present invention will readily occur to those of ordinary skill in the art in view of the disclosure herein.

### BRIEF DESCRIPTION OF THE FIGURES

FIG. 1. Administration of peptides followed by whole protein reduces the cutaneous early phase reaction to Fel d 1. In a placebo-controlled, double blind clinical trial, subjects were injected intradermally with whole Fel d 1 protein and the size of the reaction at 15 minutes measured (baseline). A series, of injections were administered to test subjects, wherein the injections were carried out using compositions of peptides derived from the amino acid sequence of Fel d 1. The dose of peptides started at 5 μg of each peptide and increased until a cumulative dose of 90 μg had been administered. Approximately 2-4 weeks later the whole Fel d 1 protein was injected (whole protein). 3-6 months later whole protein challenge was performed and the magnitude of the skin reaction at 15 minutes defined (outcome).

FIG. 2. Administration of peptides followed by whole protein reduces the cutaneous late-phase reaction to Fel d 1. In a placebo-controlled, double blind clinical trial, subjects were injected intradermally with whole Fel d 1 protein and the size of the reaction at 6 hours measured (baseline). A series of injections were administered to test subjects, wherein the injections were carried out using compositions of peptides derived from the amino acid sequence of Fel d 1. The dose of peptides started at 5 μg of each peptide and increased until a cumulative dose of 90 μg had been administered. Approximately 2-4 weeks later the whole Fel d 1 protein was injected (whole protein). 3-6 months later whole protein challenge was performed and the magnitude of the skin reaction at 6 hours defined (outcome).

FIG. 3. Peptides from the cat allergen Fel d 1 were administered intradermally at 2 weekly intervals in the following dose schedule: 0.1 μg, 1.0 μg, 5.0 μg, 10.0 μg, 25.0 μg. No isolated late asthmatic reactions were observed (n=8 subjects; pooled data). The Fel d 1 peptides that were administered were:

| | |
|---|---|
| EICPAVKRDVDLFLTGT; | (SEQ ID NO. 1) |
| LFLTGTPDEYVEQVAQY; | (SEQ ID NO. 2) |
| EQVAQYKALPVVLENA; | (SEQ ID NO. 3) |
| KALPVVLENARILKNCV; | (SEQ ID NO. 4) |
| RILKNCVDAKMTEEDKE; | (SEQ ID NO. 5) |
| KMTEEDKENALSLLDK; | (SEQ ID NO. 6) |
| KENALSLLDKIYTSPL; | (SEQ ID NO. 7) |
| LTKVNATEPERTAMKK; | (SEQ ID NO. 8) |
| TAMKKIQDCYVENGLI; | (SEQ ID NO. 9) |
| SRVLDGLVMTTISSSK; | (SEQ ID NO. 10) |
| ISSSKDCMGEAVQNTV; | (SEQ ID NO. 11) |
| and | |
| AVQNTVEDLKLNTLGR. | (SEQ ID NO. 12) |

### DETAILED DESCRIPTION OF THE INVENTION

Before describing the present invention in detail, it is to be understood that this invention is not limited to particularly exemplified molecules, methods or process parameters as such may, of course, vary. It is also to be understood that the terminology used herein is for the purpose of describing particular embodiments of the invention only, and is not intended to be limiting. In addition, the practice of the present will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise.

### A. DEFINITIONS

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

By “needleless syringe” is meant an instrument which delivers a particulate (e.g., powdered) composition transdermally without the aid of a conventional needle to pierce the skin. Needleless syringes for use with the present invention are discussed throughout this document, and are used to carry out transdermal particle injection techniques.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Thus, the term encompasses delivery from a needleless syringe deliver as described in U.S. Pat. No. 5,630,796, as well as particle-mediated delivery as described in U.S. Pat. No. 5,865,796.

A “peptide,” used interchangeably herein with the term “polypeptide,” is used in it broadest sense to refer to a composition of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

An “antigen” refers to any agent, generally a macromolecule, which can elicit an immunological response in an individual. The term may be used to refer to an individual macromolecule or to a homogeneous or heterogeneous population of antigenic macromolecules. Antigens include allergens and autoantigens. As used herein, “antigen” is generally used to refer to a polypeptide molecule or portion thereof which contains one or more epitopes. For purposes of the present invention, antigens can be obtained or derived from any appropriate source. Furthermore, for purposes of the present invention, an “antigen” includes a polypeptide having modifications, such as deletions, additions and substitutions (generally conservative in nature) to the native sequence, so long as the polypeptide maintains sufficient immunogenicity. These modifications may be deliberate, for example through site-directed mutagenesis, or may be accidental, such as through mutations of hosts which produce the antigens.

In various aspects of the invention, an antigen is described as a molecule containing one or more T cell epitopes. A “T cell epitope” refers generally to those features of a peptide structure which are capable of inducing a T cell response. In this regard, it is accepted in the art that T cell epitopes comprise linear peptide determinants that assume extended conformations within the peptide-binding cleft of MHC molecules, (Unanue et al. (1987) Science 236:551-557). As used herein, a T cell epitope is generally a peptide having at least about 3-5 amino acid residues, and preferably at least 5-10 or more amino acid residues. The ability of a particular antigen to stimulate a cell-mediated immunological response may be determined by a number of well-known assays, such as by lymphoproliferation (lymphocyte activation) assays, CTL cytotoxic cell assays, or by assaying for T-lymphocytes specific for the antigen in a sensitized subject. See, e.g., Erickson et al. (1993) J. Immunol. 151:4189-4199; and Doe et al. (1994) Eur. J. Immunol. 24:2369-2376:

An “allergen” is an antigen which can initiate a state of hypersensitivity or which can provoke an immediate hypersensitivity reaction in an individual already sensitized with the allergen. Allergens are commonly proteins or chemicals bound to proteins which have the property of being allergenic; however, allergens can also include organic or inorganic materials derived from a variety of man-made or natural sources such as plant materials, metals, ingredients in cosmetics or detergents, latexes, or the like. Allergens can elicit any type of hypersensitivity reaction in a sensitized individual. For example, penicillin allergies can manifest as all four types (Type I-IV) of hypersensitivity reactions, contact dermatitis can manifest as a Type IV reaction, and gluten allergy can manifest as a Type III reaction. However, allergens typically are associated with Type I immediate hypersensitivity reactions in sensitized individuals.

Protein or peptide allergens are typically molecules that comprise a region derived or obtained from known sources, including but not limited to protein allergens specific for the genus Dermatophagoides; the genus Euroglyphus; the genus Felis; the genus Ambrosia; the genus Lolium; the genus Phleum; the genus Cryptomeria; the genus Alternaria; the genus Alnus; the genus Betula; the genus Carpinus; the genus Quercus: the genus Olea; the genus Artemisia; the genus Plantago; the genus Parietaria; the genus Canine; the genus Blattella, the genus Apis; the genus Rubisco; the genus Vespula; and the genus Periplaneta.

Techniques for determining nucleic acid and amino acid “sequence identity” or “sequence homology” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman (1981) Advances in Applied Mathematics 2:482-489. This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986) Nucl. Acids Res. 14(6):6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5×Denhardt=s Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

As used herein, the term “autoimmune disease” means a set of sustained organ-specific or systemic clinical symptoms and signs associated with altered immune homeostasis that is manifested by qualitative and/or quantitative defects of expressed autoimmune repertoires (Autoimmunity Physiology and Disease, Coutinho and Kazatchkine, eds, Wiley-Liss, 1993, Chapter 27, page 433). Autoimmune diseases are characterized by cytotoxic immune responses to epitopes on self antigens natively found in the diseased individual. The immune system of the individual then activates an inflammatory cascade aimed at cells and tissues presenting those specific self antigens. The destruction of the antigen, tissue, cell type, or organ attacked by the individual's own immune system gives rise to the symptoms of the disease. Clinically significant autoimmune diseases include, for example, rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura. See e.g., Paul, W. E. (1993) Fundamental Immunology, Third Edition, Raven Press, New York, Chapter 30, pp. 1033-1097; and Cohen et al. (1994) Autoimmune Disease Models, A Guidebook, Academic Press, 1994.

The term “self antigen,” which is used interchangeably herein with the term “autoantigen,” means an antigen, or a molecule capable of being recognized during an immune response, that is normally part of the individual. This is in contrast with antigens which are foreign, or exogenous, which are not normally part of the individual's milieu. Each autoimmune disease is characterized by an immune response directed at a self antigen. Normally, there are no active immune responses to self antigens, and no symptoms appear. With the development of an immune response to a self antigen, autoimmune diseases may appear. Autoimmune diseases present clinically with different symptoms depending upon the specific self antigen against which an immune response is raised. This immune response results in the destruction of the structure containing the self antigen, and it is the loss of that structure with concurrent loss of that structure's normal function which results in symptoms of autoimmune disease.

Compositions that comprise an antigen or allergen are typically prepared as pharmaceutical compositions which can contain one or more added materials such as carriers, vehicles, and/or excipients. “Carriers,” “vehicles” and “excipients” generally refer to substantially inert materials which are non-toxic, not pharmacologically active and do not interact with other components of the composition in a deleterious manner. These materials can be used to increase the amount of solids in particulate compositions. Examples of suitable carriers or vehicles include water, silicone, gelatin, waxes, and like materials. Examples of normally employed excipients include pharmaceutical grades of dextrose, sucrose, lactose, trehalose, mannitol, sorbitol, inositol, dextran, starch, cellulose, sodium or calcium phosphates, calcium carbonate, calcium sulfate, sodium citrate, citric acid, tartaric acid, glycine, high molecular weight polyethylene glycols (PEG), and combinations thereof.

The terms “individual” and “subject” are used interchangeably herein to refer to any member of the subphylum cordata, including, without limitation, humans and other primates, including non-human primates such as chimpanzees and other apes and monkey species; mammals including for example farm animals such as cattle, sheep, pigs, goats and horses, domestic mammals such as dogs and cats, and laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

### B. GENERAL OVERVIEW

There exists a need for alternative therapeutic strategies to desensitise subjects to polypeptide antigens, particularly allergens. In particular, since many individuals are allergic, or may require desensitising to several polypeptide antigens, it would be beneficial to provide systems that allow for desensitisation to multiple polypeptide antigens, and in particular multiple allergens.

More particularly, we have found, surprisingly, that “tolerance” induced in an individual to a first polypeptide antigen or allergen can create in the individual a “tolergeneic environment” wherein inappropriate immune responses to other antigens can be downregulated in order to provide tolerance to other antigens. This surprising finding means that individuals allergic to multiple allergens can be treated in a greatly reduced time period, and that individuals seriously allergic to some allergens (e.g., peanuts) but more mildly allergic to other allergens (e.g., cat dander) can benefit from a therapy wherein tolerance to the milder allergen is established and then this tolergeneic environment is used to provide tolerance to the other, more extreme allergen. In addition, individuals suffering from an autoimmune disorder who are additionally sensitised (or otherwise immune) to an unrelated antigen or allergen can benefit from a treatment regime wherein tolerance to the unrelated antigen or allergen is first established and then this tolergeneic environment is used to provide tolerance to the autoantigen associated with the autoimmune disorder.

Accordingly, in a first aspect of the invention we provide a method of desensitising an individual to one or more polypeptide antigens each of which contains a T cell epitope, the method comprising: step (1) administering to the individual a T-cell-epitope-containing peptide, or a course of T-cell-epitope-containing peptides, of a first antigen to which the individual has been previously exposed, in order to generate a state of hyporesponsiveness to the first antigen; then step (2) administering to the individual a compound, that is, a composition that comprises the T cell epitope of a peptide administered in step (1) and optionally further comprising a T cell epitope of the one or more polypeptide antigens to which the individual is to be desensitised; and, if the composition administered in step (2) does not contain a T cell epitope of the said one or more polypeptide antigens, the method further comprises the step of (3) administering to the individual a composition which contains a T cell epitope of the one or more polypeptide antigens to which the individual is to be desensitised, wherein in steps (2) and (3) the composition is substantially localised at the site of administration and if steps (2) and (3) are carried out the compositions are administered to substantially the same site.

Preferably, the first antigen is an allergen. In addition, the individual to be treated is typically sensitised to the antigen in the sense that the individual displays a pathogenic recognition of the first antigen.

Put in another way, in a first aspect of the invention, a method is provided for desensitising an individual to one or more polypeptide antigens. The method entails, in a first step, administering to the individual a primary composition comprising a first polypeptide antigen containing a T cell epitope, wherein the individual has been previously exposed to the antigen and the administration is carried out in a manner sufficient to generate a hyporesponsive state against the first polypeptide antigen. Once a hyporesponsive state has been established toward the first polypeptide antigen, or at least a shift toward desensitisation has occurred, the method entails administration of a secondary composition comprising a second, different polypeptide antigen to which the individual is to be sensitised. Administration of the secondary composition is carried out in such a way as to take advantage of the tolergeneic environment established by use of the primary composition, where it is now possible to establish tolerance to the second, different polypeptide antigen. The secondary composition is coadministered with either the first polypeptide antigen or a larger molecule containing the first polypeptide antigen. By “coadministered” it is meant either the simultaneous or concurrent administration of polypeptide antigens, e.g., when the two are present in the same composition or administered in separate compositions at nearly the same time but at different sites, as well as the delivery of polypeptide antigens in separate compositions at different times. For example, the secondary composition may be delivered prior to or subsequent to delivery of the first polypeptide antigen (or a larger molecule comprising the first polypeptide antigen) at the same or a different site. The timing between deliveries can range from about several seconds apart to about several minutes apart, several hours apart, or even several days apart. Furthermore, different delivery methods can be employed.

The first polypeptide antigen is preferably an allergen to which the individual is sensitised to. In addition, in some embodiments, the second polypeptide antigen is also an allergen, albeit a second, different allergen. Suitable allergens for use in the methods of the invention can of course be obtained and/or produced using known methods. Classes of suitable allergens include, but are not limited to, pollens, animal dander, grasses, molds, dusts, antibiotics, stinging insect venoms, and a variety of environmental (including chemicals and metals), drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (dermatophagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common environmental allergens include metals (nickel and gold), chemicals (formaldehyde, trinitrophenol and turpentine), Latex, rubber, fiber (cotton or wool), burlap, hair dye, cosmetic, detergent and perfume allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der p I allergen (Hoyne et al. (1994) Immunology 83190-195), bee venom phospholipase A2 (PLA) (Akdis et al. (1996) J. Clin. Invest. 98:1676-1683), birch pollen allergen Bet v 1 (Bauer et al. (1997) Clin. Exp. Immunol. 107:536-541), and the multi-epitopic recombinant grass allergen rKBG83 (Cao et al. (1997) Immunology 90:46-51). These and other suitable allergens are commercially available and/or can be readily prepared as extracts following known techniques.

In preferred embodiments, the allergen used in the primary composition is a Fel d 1 (the feline skin and salivary gland allergen of the domestic cat Felis domesticus—the amino acid sequence of which is disclosed in International Publication WO 91/06571); Der p I, Der p II, Der II or Der fII (the major protein allergens from the house dust mite dermatophagoides—amino acid sequences disclosed in International Publication WO 94/24281); or allergens present in any of the following: grass, tree and weed (including ragweed) pollens; fungi and moulds; foods (e.g., fish, shellfish, crab, lobster, peanuts, nuts, wheat gluten, eggs and milk); stinging insects e.g., bee, wasp and hornet and the chirnomidae (non-biting midges); spiders and mites, including the house dust mite; allergens found in the dander, urine, saliva, blood or other bodily fluid of mammals such as cat, dog, cows, pigs, sheep, horse, rabbit, rat, guinea pig, mouse and gerbil; airborne particulates in general; latex; and protein detergent additives. Where the allergen is an insect protein, the peptides may be selected from any of housefly, fruit fly, sheep blow fly, screw worm fly, grain weevil, silkworm, honeybee, non-biting midge larvae, bee moth larvae, mealwomm, cockroach and larvae of Tenibrio molitor beetle. All these being insect allergens, they are of particular relevance to allergic problems arising in the workplace.

It is preferred that the allergen present in the primary composition is an aero allergen (i.e., an airborne allergen) since it is likely that the individual has previously been exposed to such an allergen even if he or she is not allergic to the allergen. Thus, particularly preferred allergens are those from cat or dog dander, those from pollen, those from fungi and moulds, those from house dust mite or other mites and those from certain foods such as nuts, especially peanuts. Latex can be an aero allergen as can peanut allergen when an aerosol is created, for example when frying in peanut oil.

A particularly preferred allergen for use in the methods described herein, particularly the allergen present in the primary composition, is Fel d 1. Thus, in step (1) it is preferred that one or more T-cell-epitope-containing peptides of Fel d 1 are administered to the individual. It is particularly preferred if one or more of the following peptides of Fel d 1 are administered: EQVAQYKALPVVLENA (SEQ ID NO. 2) or KALPVVLENARILKNCV (SEQ ID NO. 3), both of which are known to be MHC restricted. These peptides are described in more detail in commonly owned International Publication WO 99/34826 (PCT/GB99/00080). Other allergens useful in the present methods can be readily produced using publicly available sequence information for the allergens. The following is a list of known allergen sequences and database accession numbers (NCBI Entrez accession numbers). NCBI is the National Center for Biotechnology information and is a division of the US National Institutes of Health. The NCBI web site, from which access to the database may be sought, www.ncbi.nlm.nih.gov/. The allergens may be used as described above in order to identify MHC-restricted peptides capable of inducing LPR in individuals who possess a particular MHC molecule.

Allergen sequences and database accession numbers (NCBI Entrez accession numbers):

House dust mite
Dermatophagoides pteronyssinus
Der p 1
MKIVLAIASLLALSAVYARPSSIKTFEEYKKAFNKSYATFEDEEAARK
NFLESVKYVQSNGGAINHLSDLSLDEFKNRFLMSAEAFEHLKTQFDLN
AETNACSINGNAPAEIDLRQMRTVTPIRMQGGCGSCWAFSGVAATESA
YLAYRNQSLDLAEQELVDCASQHGCHGDTIPRGIEYIQHNGVVQESYY
RYVAREQSCRRPNAQRFGISNYCQIYPPNVNKIREALAQTHSAIAVII
GIKDLDAFRHYDGRTIIQRDNGYQPNYHAVNIVGYSNAQGVDYWIVRN
SWDTNWGDNGYGYFAANIDLMMIEEYPYVVIL
Der p 2
MMYKILCLSLLVAAVARDQVDVKDCANHEIKKVLVPGCHGSEPCIIHR
GKPFQLEAVFEANQNTKTAKIEIKASIDGLEVDVPGIDPNACHYMKCP
LVKGQQYDIKYTWNVPKIAPKSENVVVTVKVMGDDGVLACAIATHAKI
RD
Der p 3
MIIYNILIVLLLAINTLANPILPASPNATIVGGEKALAGECPYQISLQ
SSSHFCGGTILDEYWILTAAHCVAGQTASKLSIRYNSLKHSLGGEKIS
VAKIFAHEKYDSYQIDNDIALIKLKSPMKLNQKNAKAVGLPAKGSDVK
VGDQVRVSGWGYLEEGSYSLPSELRRVDIAVVSRKECNELYSKANAEV
TDNMICGGDVANGGKDSCQGDSGGPVVDVKNNQVVGIVSWGYGCARKG
YPGVYTRVGNFIDWIESKRSQ
Der p 4
KYXNPHFIGXRSVITXLME
Der p 5
MKFIIAFFVATLAVMTVSGEDKKHDYQNEFDFLLMERIHEQIKKGELA
LFYLQEQINHFEEKPTKEMKDKIVAEMDTIIAMIDGVRGVLDRLMQRK
DLDIFEQYNLEMAKKSGDILERDLKKEEARVKKIEV
Der p 6
AIGXQPAAEAEAPFQISLMK
Der p 7
MMKLLLIAAAAFVAVSADPIHYDKITEEINKAVDEAVAAIEKSETFDP
MKVPDHSDKFERHIGIIDLKGELDMRNIQVRGLKQMKRVGDANVKSED
GVVKAHLLVGVHDDVVSMEYDLAYKLGDLHPNTHVISDIQDFVVELSL
EVSEEGNMTLTSFEVRQFANVVNHIGGLSILDPIFAVLSDVLTAIFQD
TVRAEMTKVLAPAFKKELERNNQ
Der p9
IVGGSNASPGDAVYQIAL
Dermatophagoides farinae
Der f 1
MKFVLAIASLLVLTVYARPASIKTFEFKKAFNKNYATVEEEEVARKNF
LESLKYVEANKGAINHLSDLSLDEFKNRYLMSAEAFEQLKTQFDLNAE
TSACRINSVNVPSELDLRSLRTVTPIRMQGGCGSCWAFSGVAATESAY
LAYRNTSLDLSEQELVDCASQHGCHGDTIPRGIEYIQQNGVVEERSYP
YVAREQRCRRPNSQHYGISNYCQIYPPDVKQIREALTQTHTAIAVIIG
IKDLRAFQHYDGRTIIQHDNGYQPNYHAVNIVGYGSTQGDDYWIVRNS
WDTTWGDSGYGYFQAGNNLMMIEQYPYVVIM
Der f 2
MISKILCLSLLVAAVVADQVDVKDCANNEIKKVMVDGCHGSDPCIIHR
GKPFTLEALFDANQNTKTAKIEIKASLDGLEIDVPGIDTNACHFMKCP
LVKGQQYDIKYTWNVPKIAPKSENVVVTVKLIGDNGVLACAIATHGKI
RD
Der f 3
MMILTIVVLLAANILATPILPSSPNATIVGGVKAQAGDCPYQISLQSS
SHFCGGSILDEYWILTAAHCVNGQSAKKLSIRYNTLKHASGGEKIQVA
EIYQHENYDSMTIDNDVALIKLKTPMTLDQTNAKPVPLPAQGSDVKVG
DKIRVSGWGYLQEGSYSLPSELQRVDIDVVSREQCDQLYSKAGADVSE
NMICGGDVANGGVDSCQGDSGGPVVDVATKQIVGIVSWGYGCARKGYP
GVYTRVGNFVDWIESKRSQ
Der f 4
AVGGQDADLAEAPFQISLLK
Der f 7
MMKFLLIAAVAFVAVSADPIHYDKITEEINKAIDDAIAAIEQSETIDP
MKVPDHADKFERHVGIVDFKGELAMRNIEARGLKQMKRQGDANVKGEE
GIVKAHLLIGVHDDIVSMEYDLAYKLGDLHPTTHVISDIQDFVVALSL
EISDEGNITMTSFEVRQFANVVNHIGGLSILDPIFGVLSDVLTAIFQD
TVRKEMTKVLAPAFKRELEKN

### Additional Mite Allergen Sequences (NCBI Entrez Accession):

1170095; 1359436; 2440053; 666007; 487661; 1545803; 84702; 84699; 625532; 404370; 1091577; 1460058; 7413; 9072; 387592.

Cat
Felis sequences
1082946 Fel dI chain 2 precursor - cat
MRGALLVLALLVTQALGVKMAETCPIFYDVFFAVANGNELLLDLSLTKV
NATEPERTAMKKIQDCYVENGLISRVLDGLVMTTISSSKDCMGEAVQNTV
EDLKLNTLGR
1082945 Fel dI chain 1 short form - cat
MLDAALPPCPTVAATADCEICPAVKRDVDLFLTGTPDEYVEQVAQYKALP
VVLENARILKNCVDAKMTEEDKENALSLLDKIYTSPLC
1082944 Fel dI chain 1 long form precursor -
cat
MKGARVLVLLWAALLLIWGGNCEICPAVKRDVDLFLTGTPDEYVEQVAQ
YKALPVVLENARILKNCVDAKMTEEDKENALSLLDKIYTSPLC

### Additional Felis Sequences (NCBI Entrez Accession):

539716; 539715; 423193; 423192; 423191; 423190; 1364213; 1364212; 395407; 163827; 163823; 163825; 1169665; 232086; 1169666.

Latex
Hevea sequences:
Hev b 1
MAEDEDNQQGQGEGLKYLGFVQDAATYAVTTFSNVYLFAKDKSGPLQP
GVDIIEGPVKNVAVPLYNRFSYLPNGALKFVDSTVVASVTIIDRSLPPIV
KDASIQVVSAIRAAPEAARSLASSLPGQTKILAKVFYGEN
Hev b 3
MAEEVEEERLKYLDFVRAAGVYAVDSFSTLYLYAKDISGPLKPGVDTIEN
VVKTVVTPVYYIPLEAVKFVDKTVDVSVTSLDGVVPPVIKQVSAQTYSVA
QDAPRIVLDVASSVFNTGVQEGAKALYANLEPKAEQYAVITWRALNKLP
LVPQVANVVVPTAVYFSEKYNDVVRGTTEQGYRVSSYLPLLPTEKITKVF
GDEAS

### Additional Hevea Sequences (NCBI Entrez Accession):

3319923; 3319921; 3087805; 1493836; 1480457; 1223884; 3452147; 3451147; 1916805; 232267; 123335; 2501578; 3319662; 3288200; 1942537; 2392631; 2392630; 1421554; 1311006; 494093; 3183706; 3172534; 283243; 1170248; 1708278; 1706547; 464775; 266892; 231586; 123337; 116359; 123062; 2213877; 542013; 2144920; 1070656; 2129914; 2129913; 2129912; 100135; 82026; 1076559; 82028; 82027; 282933; 280399; 100138; 1086972; 108697; 1086976; 1086978; 1086978; 1086976; 1086974; 1086972; 913758; 913757; 913756; 234388; 1092500; 228691; 1177405; 18839; 18837; 18835; 18833; 18831; 1209317; 1184668; 168217; 168215; 168213; 168211; 168209; 348137.

Rye grass
Lolium sequences:
126385 Lol p 1
MASSSSVLLVVALFAVFLGSAHGIAKVPPGPNITAEYGDKWLDAKST
WYGKPTGAGPKDNGGACGYKNVDKAPFNGMTGCGNTPIFKDGRGCGS
CFEIKCTKPESCSGEAVTVTITDDNEEPIAPYHFDLSGHAFGSMAKK
GEEQNVRSAGELELQFRRVKCKYPDDTKPTFHVEKASNPNYLAILVK
YVDGDGDVVAVDIKEKGKDKWIELKESWGAVWRIDTPDKLTGPFTVR
YTTEGGTKSEFEDVIPEGWKADTSYSAK
126386 Lol p 2a
AAPVEFTVEKGSDEKNLALSIKYNKEGDSMAEVELKEHGSNEWLALK
KNGDGVWEIKSDKPLKGPFNFRFVSEKGMRNVFDDVVPADFKVGTTY
KPE
126387 Lol p 3
TKVDLTVEKGSDAKTLVLNIKYTRPGDTLAEVELRQHGSEEWEPMTK
KGNLWEVKSAKPLTGPMNFRFLSKGGMKNVFDEVIPTAFTVGKTYTP
EYN
2498581 Lol p 5a
MAVQKYTVALFLRRGPRGGPGRSYAADAGYTPAAAATPATPAATPAG
GWREGDDRRAEAAGGRQRLASRQPWPPLPTPLRRTSSRSSRPPSPSP
PRASSPTSAAKAPGLIPKLDTAYDVAYKAAEAHPRGQVRRLRHCPHR
SLRVIAGALEVHAVKPATEEVLAAKIPTGELQIVDKIDAAFKIAATA
ANAAPTNDKFTVFESAFNKALNECTGGAMRPTSSSPPSRPRSSRPTP
PPSPAAPEVKYAVFEAALTKAITAMTQAQKAGKPAAAAATAAATVAT
AAATAAAVLPPPLLVVQSLISLLIYY
2498582 Lol p 5b
MAVQKHTVALFLAVALVAGPAASYAADAGYAPATPATPAAPATAATP
ATPATPATPAAVPSGKATTEEQKLIEKINAGFKAAVAAAAVVPPADK
YKTFVETFGTATNKAFVEGLASGYADQSKNQLTSKLDAALKLAYEAA
QGATPEAKYDAYVATLTEALRVIAGTLEVHAVKPAAEEVKVGAIPAA
EVQLIDKVDAAYRTAATAANAAPANDKFTVFENTFNNAIKVSLGAAY
DSYKFIPTLVAAVKQAYAAKQATAPEVKYTVSETALKKAVTAMSEAE
KEATPAAAATATPTPAAATATATPAAAYATATPAAATATATPAAATA
TPAAAGGYKV
455288 Lol p isoform 9
MAVQKHTVALFLAVALVAGPAASYAADAGYAPATPATPAAPATAATP
ATPATPATPAAVPSGKATTEEQKLIEKINAGFKAAVAAAAVVPPADK
YKTFVETFGTATNKAFVEGLASGYADQSKNQLTSKLDAALKLAYEAA
QGATPEAKYDAYVATLTEALRVIAGTLEVHAVKPAAEEVKVGAIPAA
EVQLIDKVDAAYRTAATAANAAPANDKFTVFENTFNNALKVSLGAAY
DSYKFIPTLVAAVKQAYAAKQATAPEVKYTVSETALKKAVTAMSEAE
KEATPAAAATATPTPAAATATATPAAAYATATPAAATATATPAAATA
TPAAAGGYKV
1582249 Lol p 11
DKGPGFVVTGRVYCDPCRAGFETNVSHNVEGATVAVDCRPFDGGESK
LKAEATTDKDGWYKIEIDQDHQEEICEVVLAKSPDKSCSEIEEFRDR
ARVPLTSNXGIKQQGIRYANPIAFFRKEPLKECGGILQAY

### Additional Lolium Sequences (NCBI Entrez Accession):

135480; 417103; 687261; 687259; 1771355; 2388662; 631955; 542131; 542130; 542129; 100636; 626029; 542132; 320616; 320615; 320614; 100638; 100634; 82450; 626028; 100639; 283345; 542133; 1771353; 1763163; 1040877; 1040875; 250525; 551047; 515377; 510911; 939932; 439950; 2718; 168316; 168314; 485371; 2388664; 2832717; 2828273; 548867.

Olive tree
Olive sequences
416610 Ole e 1
EDIPQPPVSQFHIQGQVYCDTCRAGFITELSEFIPGASLRLQCKDKEN
GDVTFTEVGYTRAEGLYSMLVE
RDHKNEFCEITLISSGRKDCNEIPTEGWAKPSLKFKLNTVNGTTRTVN
PLGFFKKEALPKCAQVYNKLGM
YPPNM
Parietaria
Parietaria sequences:
2497750 Par j P2
MRTVSMAALVVIAAALAWTSSAEPAPAPAPGEEACGKVVQDIMPCLHF
VKGEEKEPSKECCSGTKKLSEEVKTTEQKREACKCIVRATKGISGIKN
ELVAEVPKKCDIKTTLPPITADFDCSKIQSTIFRGYY
1352506 Par j P5
MVRALMPCLPFVQGKEKEPSKGCCSGAKRLDGETKTGPQRVHACECIQ
TAMKTYSDIDGKLVSEVPKHCGIVDSKLPPIDVNMDCKTVGVVPRQPQ
LPVSLRHGPVTGPSDPAHKARLERPQIRVPPPAPEKA
1532056 Par j P8
MRTVSMAALVVIAAALAWTSSAELASAPAPGEGPCGKVVHHIMPCLKF
VKGEEKEPSKSCCSGTKKLSEEVKTTEQKREACKCIVAATKGISGIKN
ELVAEVPKKCGITTTLPPITADFDCSKIESTIFRGYY
1532058 Par j P9
MRTVSAPSAVALVVIVAAGLAWTSLASVAPPAPAPGSEETCGTVVRAL
MPCLPFVQGKEKEPSKGCCSGAKRLDGETKTGLQRVHACECIQTAMKT
YSDIDGKLVSEVPKHCGIVDSKLPPIDVNMDCKTLGVVPRQPQLPVSL
RHGPVTGPSDPAHKARLERPQIRVPPPAPEKA
2497749 Par j P9
MRTVSARSSVALVVIVAAVLVWTSSASVAPAPAPGSEETCGTVVGALM
PCLPFVQGKEKEPSKGCCSGAKRLDGETKTGPQRVHACECIQTAMKTY
SDIDGKLVSEVPKHCGIVDSKLPPIDVNMDCKTLGVLHYKGN
1086003 Par j 1
MVRALMPCLPFVQGKEKEPSKGCCSGAKRLDGETKTGPQRVHACECIQ
TAMKTYSDIDGKLVSEVPKHCGIVDSKLPPIDVNMDCKTVGVVPRQPQ
LPVSLRHGPVTGPSRSRPPTKHGWRDPRLEFRPPHRKKPNPAFSTLG

### Additional Parietaria Sequences (NCBI Entrez Accession):

543659; 1836011; 1836010; 1311513; 1311512; 1311511; 1311510; 1311509; 240971.

Timothy grass
Phleum sequences:
Phl p 1
MASSSSVLLVVVLFAVFLGSAYGIPKVPPGPNITATYGDKWLDAKSTW
YGKPTGAGPKDNGGACGYKDVDKPPFSGMTGCGNTPIFKSGRGCGSCF
EIKCTKPEACSGEPVVVHITDDNEEPIAPYHFDLSGHAFGAMAKKGDE
QKLRSAGELELQFRRVKCKYPEGTKVTFHVEKGSNPNYLALLVKYVNG
DGDVVAVDIKEKGKDKWIELKESWGAIWRIDTPDKLTGPFTVRYTTEG
GTKTEAEDVIPEGWKADTSYESK
Phl p 1
MASSSSVLLVVALFAVFLGSAHGIPKVPPGPNITATYGDKWLDAKSTW
YGKPTAAGPKDNGGACGYKDVDKPPFSGMTGCGNTPIFKSGRGCGSCF
EIKCTKPEACSGEPVVVHITDDNEEPIAAYHFDLSGIAFGSMAKKGDE
QKLRSAGEVEIQFRRVKCKYPEGTKVTFHVEKGSNPNYLALLVKFSGD
GDVVAVDIKEKGKDKWIALKESWGAIWRIDTPEVLKGPFTVRYTTEGG
TKARAKDVIPEGWKADTAYESK
Phlp 2
MSMASSSSSSLLAMAVLAALFAGAWCVPKVTFTVEKGSNEKHLAVLVK
YEGDTMAEVELREHGSDEWVAMTKGEGGVWTFDSEEPLQGPFNFRFLT
EKGMKNVFDDVVPEKYTIGATYAPEE
Phl p 5
ADLGYGGPATPAAPAEAAPAGKATTEEQKLIEKINDGFKAALAAAAGV
PPADKYKTFVATFGAASNKAFAEGLSAEPKGAAESSSKAALTSKLDAA
YKLAYKTAEGATPEAKYDAYVATLSEALRIIAGTLEVHAVKPAAEEVK
VIPAGELQVIEKVDSAFKVAATAANAAPANDKFTVFEAAFNNAIKAST
GGAYESYKFIPALEAAVKQAYAATVATAPEVKYTVFETALKKAFTAMS
EAQKAAKPATEATATATAAVGAATGAATAATGGYKV
Phl p 5
ADLGYGGPATPAAPAEAAPAGKATTEEQKLIEKINDGFKAALAAAAGV
PPADKYKTFVATFGAASNKAFAEGLSAEPKGAAESSSKAALTSKLDAA
YKLAYKTAEGATPEAKYDAYVATLSEALRIIAGTLEVHAVKPAAEEVK
VIPAGELQVIEKVDSAFKVAATAANAAPANDKFTVFEAAFNNAIKAST
GGAYESYKFIPALEAAVKQAYAATVATAPEVKYTVFETALKKAITAMS
EAQKAAKPATEATATATAAVGAATGAATAATGGYKV
Phl p 5b
AAAAVPRRGPRGGPGRSYTADAGYAPATPAAAGAAAGKATTEEQKLIE
DINVGFKAAVAAAASVPAADKFKTFEAAFTSSSKAAAAKAPGLVPKLD
AAYSVAYKAAVGATPEAKFDSFVASLTEALRVIAGALEVHAVKPVTEE
PGMAKIPAGELQIIDKIDAAFKVAATAAATAPADDKFTVFEAAFNKAI
KESTGGAYDTYKCIPSLEAAVKQAYAATVAAAPQVKYAVFEAALTKAI
TAMSEVQKVSQPATGAATVAAGAATTAAGAASGAATVAAGGYKV
Phl p 5a
ADLGYGPATPAAPAAGYTPATPAAPAGADAAGKATTEEQKLIEKINAG
FKAALAGAGVQPADKYRTFVATFGPASNKAFAEGLSGEPKGAAESSSK
AALTSKLDAAYKLAYKTAEGATPEAKYDAYVATLSEALRIIAGTLEVH
AVKPAAEEVKVIPAGELQVIEKVDAAFKVAATAANAAPANDKFTVFEA
AFNDEIKASTGGAYESYKFIPALEAAVKQAYAATVATAPEVKYTVFET
ALKKAITAMSEAQKAAKPAAAATATATAAVGAATGAATAATGGYKV
Phl p 5
MAVQKYTVALFLAVALVAGPAASYAADAGYAPATPAAAGAEAGKATTE
EQKLIEDINVGFKAAVAAAASVPAADKFKTFEAAFTSSSKAATAKAPG
LVPKLDAAYSVSYKAAGATPEAKFDSFVASLTEALRVIAGALEVHAVK
PVTEEPGMAKIPAGELQIIDKIDAAFKVAATAAATAPADTVFEAAFNK
AIKESTGGAYDTYKCIPSLEAAVKQAYAATVAAAPQVKYAVFEAALTK
AITAMSEVQKVSQPATGAATVAAGAATTAAGAASGAATVAAGGYKV
Phl p 5
MAVQKYTVALFLAVALVAGPAASYAADAGYAPATPAAAGAEAGKATTE
EQKLIEDINVGFKAAVAAAASVPAADKFKTFEAAFTSSSKAATAKAPG
LVPKLDAAYSVAYKAAVGATPEAKFDSFVASLTEALRVIAGALEVHAV
KPVTEDPAWPKIPAGELQIIDKIDAAFKVAATAAATAPADDKFTVFEA
AFNKAIKESTGGAYDTYKCIPSLEAAVKQAYAATVAAAPQVKYAVFEA
ALTKAITAMSEVQKVSQPATGAATVAAGAATTATGAASGAATVAAGGY
KV
Phl p 5
ADAGYAPATPAAAGAEAGKATTEEQKLIEDINVGFKAAVAAAASVPAA
DKFKTFEAAFTSSSKAATAKAPGLVPKLDAAYSVAYKAAVGATPEAKF
DSFVASLTEALRVIAGALEVHAVKPVTEEPGMAKIPAGELQIIDKIDA
AFKVAATAAATAPADDKFTVFEAAFNKAIKESTGGAYDTYKCIPSLEA
AVKQAYAATVAAAPQVKYAVFEAALTKAITAMSEVQKVSQPATGAATV
AAGAATTAAGAASGAATVAAGGYKV
Phl p 5
SVKRSNGSAEVHRGAVPRRGPRGGPGRSYAADAGYAPATPAAAGAEAG
KATTEEQKLIEDINVGFKAAVAAAASVPAADKFKTFEAAFTSSSKAAT
AKAPGLVPKLDAAYSVAYKAAVGATPEAKFDSFVASLTEALRVIAGAL
EVHAVKPVTEEPGMAKIPAGELQIIDKMAAFKVAATAAATAPADDKFT
VFEAAFNKAIKESTGGAYDTYKCIPSLEAAVKQAYAATVAAAPQVKYA
VFEAALTKAITAMSEVQKVSQPATGAATVAAGAATTAAGAASGAATVA
AGGYKV
Phl p 5
MAVHQYTVALFLAVALVAGPAGSYAADLGYGPATPAAPAAGYTPATPA
APAGAEPAGKATTEEQKLIEKINAGFKAALAAAAGVPPADKYRTFVAT
FGAASNKAFAEGLSGEPKGAAESSSKAALTSKLDAAYKLAYKTAEGAT
PEAKYDAYVATVSEALRIIAGTLEVHAVKPAAEEVKVIPAGELQVIEK
VDAAFKVAATAANAAPANDKFTVFEAAFNDAIKASTGGAYESYKFIPA
LEAAVKQAYAATVATAPEVKYTVFETALKKAITAMSEAQKAAKPAAAA
TATATAAVGAATGAATAATGGYKV
Phl p 5
ADLGYGGPATPAAPAEAAPAGKATTEEQKLIEKINDGFKAALAAAAGV
PPADKYKTFVATFGAASNKAFAEGLSAEPKGAAESSSKAALTSKLDAA
YKLAYKTAEGATPEAKYDAYVATLSEALRIIAGTLEVHAVKPAAEEVK
VIPAGELQVIEKVDSAFKVAATAANAAPANDKFTVFEAAFNNAIKAST
GGAYESYKFIPALEAAVKQAYAATVATAPEVKYTVFETALKKAFTAMS
EAQKAAKPATEATATATAAVGAATGAATAATGGYKV
Phl p5b
AAAAVPRRGPRGGPGRSYTADAGYAPATPAAAGAAAGKATTEEQKLIE
DINVGFKAAVAAAASVPAADKFKTFEAAFTSSSKAAAAKAPGLVPKLD
AAYSVAYKAAVGATPEAKFDSFVASLTEALRVIAGALEVHAVKPVTEE
PGMAKIPAGELQIIDKIDAAFKVAATAAATAPADDKFTVFEAAFNKAI
KESTGGAYDTYKCIPSLEAAVKQAYAATVAAAPQVKYAVFEAALTKAI
TAMSEVQKVSQPATGAATVAAGAATTAAGAASGAATVAAGGYKV
Phl p5a
ADLGYGPATPAAPAAGYTPATPAAPAGADAAGKATTEEQKLIEKINAG
FKAALAGAGVQPADKYRTFVATFGPASNKAFAEGLSGEPKGAAESSSK
AALTSKLDAAYKLAYKTAEGATPEAKYDAYVATLSEALRIIAGTLEVH
AVKPAAEEVKVIPAGELQVIEKVDAAFKVAATAANAAPANDKFTVFEA
AFNDEIKASTGGAYESYKFIPALEAAVKQAYAATVATAPEVKYTVFET
ALKKAITAMSEAQKAAKPAAAATATATAAVGAATGAATAATGGYKV
Phl p 5
AVPRRGPRGGPGRSYAADAGYAPATPAAAGAEAGKATTEEQKLIEDIN
VGFKAAVAAAASVPAGDKFKTFEAAFTSSSKAATAKAPGLVPKLDAAY
SVAYKAAVGATPEAKFDSFVASLTEALRVIAGALEVHAVKPVTEEPGM
AKIPAGELQIIDKIDAAFKVAATAAATAPADDKFTVFEAAFNKAIKES
TGGAYDTYKCIPSLEAAVKQAYAATVAAAPQVKYAVFEAALTKAITAM
SEVQKVSQPATGAATVAAGAATTATGAASGAATVAAGGYKV
Phl p 5b
MAVPRRGPRGGPGRSYTADAGYAPATPAAAGAAAGKATTEEQKLIEDI
NVGFKAAVAARQRPAADKFKTFEAASPRHPRPLRQGAGLVPKLDAAYS
VAYKAAVGATPEAKFDSFVASLTEALRVIAGALEVHAVKPVTEEPGMA
KIPAGELQIIDKIDAAFKVAATAAATAPADDKFTVFEAAFNKAIKEST
GGAYDTYKCIPSLEAAVKQAYAATVAAAAEVKYAVFEAALTKAITAMS
EVQKVSQPATGAATVAAGAATTAAGAASGAATVAAGGYKV
Phl p 5
MAVHQYTVALFLAVALVAGPAASYAADLGYGPATPAAPAAGYTPATPA
APAEAAPAGKATTEEQKLIEKINAGFKAALAAAAGVQPADKYRTFVAT
FGAASNKAFAEGLSGEPKGAAESSSKAALTSKLDAAYKLAYKTAEGAT
PEAKYDAYVATLSEALRIIAGTLEVHAVKPAAEEVKVIPAGELQVIEK
VDAAFKVAATAANAAPANDKFTVFEAAFNDAIKASTGGAYESYKFIPA
LEAAVKQAYAATVATAPEVKYTVFETALKKAITAMSEAQKAAKPAAAA
TATATAAVGAATGAATAATGGYKV
Phl p 5
EAPAGKATTEEQKLIEKINAGFKAALARRLQPADKYRTFVATFGPASN
KAFAEGLSGEPKGAAESSSKAALTSKLDAAYKLAYKTAEGATPEAKYD
AYVATLSEALRIIAGTLEVHAVKPAAEEVKVIPAAELQVIEKVDAAFK
VAATAANAAPANDKFTVFEAAFNDEIKASTGGAYESYKFIPALEAAVK
QAYAATVATAPEVKYTVFETALKKAITAMSEAQKAAKPPPLPPPPQPP
PLAATGAATAATGGYKV
Phl p 5
MAVHQYTVALFLAVALVAGPAASYAADLGYGPATPAAPAAGYTPATPA
APAEAAPAGKATTEEQKLIEKINAGFKAALAAAAGVQPADKYRTFVAT
FGAASNKAFAEGLSGEPKGAAESSSKAALTSKLDAAYKLAYKTAEGAT
PEAKYDAYVATLSEALRIIAGTLEVHAVKPAAEEVKVIPAGELQVIEK
VDAAFKVAATAANAAPANDKFTVFEAAFNDAIKASTGGAYESYKFTPA
LEAAVKQAYAATVATAPEVKYTVFETALKKAITAMSEAQKAAKPAAAA
TATATAAVGAATGAATAATGGYKV
Phl p 5b
MAVPRRGPRGGPGRSYTADAGYAPATPAAAGAAAGKATTEEQKLIEDI
NVGFKAAVAARQRPAADKFKTFEAASPRHPRPLRQGAGLVPKLDAAYS
VAYKAAVGATPEAKFDSFVASLTEALRVIAGALEVHAVKPVTEEPGMA
KIPAGELQIIDKIDAAFKVAATAAATAPADDKFTVFEAAFNKAIKEST
GGAYDTYKCIPSLEAAVKQAYAATVAAAAEVKYAVFEAALTKAITAMS
EVQKVSQPATGAATVAAGAATTAAGAASGAATVAAGGYKV
Phl p 5a
ADLGYGPATPAAPAAGYTPATPAAPAGADAAGKATTEEQKLIEKINAG
FKAALAGAGVQPADKYRTFVATFGPASNKAFAEGLSGEPKGAAESSSK
AALTSKLDAAYKLAYKTAEGATPEAKYDAYVATLSEALRIIAGTLEVH
AVKPAAEEVKVIPAGELQVIEKVDAAFKVAATAANAAPANDKFTVFEA
AFNDEIKASTGGAYESYKFIPALEAAVKQAYAATVATAPEVKYTVFET
ALKKAITAMSEAQKAAKPPPLPPPPQPPPLAATGAATAATGGYKV
Phl p 5
MAVHQYTVALFLAVALVAGPAASYAADLGYGPATPAAPAAGYTPATPA
APAEAAPAGKATTEEQKLIEKINAGFKAALAAAAGVQPADKYRTFVAT
FGAASNKAFAEGLSGEPKGAAESSSKAALTSKLDAAYKLAYKTAEGAT
PEAKYDAYVATLSEALRIIAGTLEVHAVKPAAEEVKVIPAGELQVIEK
VDAAFKVAATAANAAPANDKFTVFEAAFNDAIKASTGGAYESYKFIPA
LEAAVKQAYAATVATAPEVKYTVFETALKKAITAMSEAQKAAKPAAAA
TATATAAVGAATGAATAATGGYKV
Phl p 6
MAAHKFMVAMFLAVAVVLGLATSPTAEGGKATTEEQKLIEDVNASFRA
AMATTANVPPADKYKTFEAAFTVSSKRNLADAVSKAPQLVPKLDEVYN
AAYNAADHAAPEDKYEAFVLHFSEALRIIAGTPEVHAVKPGA
Phl p 6
SKAPQLVPKLDEVYNAAYNAADHAAPEDKYEAFVLHFSEALHIIAGTP
EVHAVKPGA
Phl p 6
ADKYKTFEAAFTVSSKRNLADAVSKAPQLVPKLDEVYNAAYNAADHAA
PEDKYEAFVLHFSEALHIIAGTPEVHAVKPGA
Phl p 6
TEEQKLIEDVNASFRAAMATTANVPPADKYKTLEAAFTVSSKRNLADA
VSKAPQLVPKLDEVYNAAYNAADHAAPEDKYEAFVLHFSEALRIIAGT
PEVHAVKPGA
Phl p 6
MAAHKFMVAMFLAVAVVLGLATSPTAEGGKATTEEQKLIEDINASFRA
AMATTANVPPADKYKTFEAAFTVSSKRNLADAVSKAPQLVPKLDEVYN
AAYNAADHAAPEDKYEAFVLHFSEALHIIAGTPEVHAVKPGA
Phl p 6
MVAMTLAVAVVLGLATSPTAEGGKATTEEQKLIEDVNASFRAAMATTA
NVPPADKYKTFEAAFTVSSKRNLADAVSKAPQLVPKLDEVYNAAYNAA
DHAAPEDKYEAFVLHFSEALRIIAGTPEVHAVKPGA
Phl p 7
MADDMERIFKRFDTNGDGKISLSELTDALRTLGSTSADEVQRMMAEID
TDGDGFIDFNEFISFCNANPGLMKDVAKVF
Phl p 11
MSWQTYVDEHLMCEIEGHHLASAAILGHDGTVWAQSADFPQFKPEEIT
GIMKDFDEPGHLAPTGMFVAGAKYMVIQGEPGRVIRGKKGAGGITIKK
TGQALVVGIYDEPMTPGQCNMVVERLGDYLVEQGM

### Additional Phleum Sequences (NCBI Entrez Accession):

458878; 548863; 2529314; 2529308; 2415702; 2415700; 2415698; 542168; 542167; 626037; 542169; 541814; 542171; 253337; 253336; 453976; 439960.

Wasp (and related)
Vespula sequences:
465054 ALLERGEN VES V 5
MEISGLVYLIIIVTIIDLPYGKANNYCKIKCLKGGVHTACKYGSLKPNCG
NKVVVSYGLTKQEKQDILKEHNDFRQKIARGLETRGNPGPQPPAKNMKNL
VWNDELAYVAQVWANQCQYGHDTCRDVAKYQVGQNVALTGSTAAKY
DDPVKLVKMWEDEVKDYNPKKKFSGNDFLKTGHYTQMVWANTKEVGC
GSIKYIQEKWHKHYLVCNYGPSGNFMNEELYQTK
1709545 ALLERGEN VES M 1
GPKCPFNSDTVSIIIETRENRNRDLYTLQTLQNHPEFKKKTITRPVVFIT
HGFTSSASEKNFINLAKALVDKDNYMVISIDWQTAACTNEYPGLKYAYYP
TAASNTRLVGQYIATITQKLVKDYKISMANIRLIGHSLGAHVSGFAGKRV
QELKLGKYSEIIGLDPARPSFDSNHCSERLCETDAEYVQIIHTSNYLGTE
KILGTVDFYMNNGKNNPGCGRFFSEVCSHTRAVIYMAECIKHECCLIGIP
RSKSSQPISRCTKQECVCVGLNAKKYPSRGSFYVPVESTAPFCNNKGKII
1352699 ALLERGEN VES V 1
MEENMNLKYLLLFVYFVQVLNCCYGHGDPLSYELDRGPKCPFNSDTVSII
IETRENRNRDLYTLQTLQNHPEFKKKTITRPVVFITHGFTSSASETNFIN
LAKALVDKDNYMVISIDWQTAACTNEAAGLKYLYYPTAARNTRLVGQYIA
TITQKLVKHYKISMANIRLIGHSLGAHASGFAGKKVQELKLGKYSEIIGL
DPARPSFDSNHCSERLCETDAEYVQIIHTSNYLGTEKTLGTVDFYMNNGK
NQPGCGRFFSEVCSHSRAVIYMAECIKHECCLIGIPKSKSSQPISSCTKQ
ECVCVGLNAKKYPSRGSFYVPVESTAPFCNNKGKII
1346323 ALLERGEN VES V 2
SERPKRVFNIYWNVPTFMCHQYDLYFDEVTNFNIKRNSKDDFQGDKIAIF
YDPGEFPALLSLKDGKYKKRNGGVPQEGNITIHLQKFIENLDKIYPNRNF
SGIGVIDFERWRPIFRQNWGNMKIHKNFSIDLVRNEHPTWNKKMIELEAS
KRFEKYARFFMEETLKLAKKTRKQADWGYYGYPYCFNMSPNNLVPECDV
TAMHENDKMSWLFNNQNVLLPSVYVRQELTPDQRIGLVQGRVKEAVRIS
NNLKHSPKVLSYWWYVYQDETNTFLTETDVKKTFQEIVINGGDGIIIWGS
SSDVNSLSKCKRLQDYLLTVLGPIAINVTEAVN
549194 ALLERGEN VES VI
5KVNYCKIKCLKGGVHTACKYGTSTKPNCGKMVVKAYGLTEAEKQEILK
VHNDFRQKVAKGLETRGNPGPQPPAKNMNNLVWNDELANIAQVWASQC
NYGHDTCKDTEKYPVGQNIAKRSTTAALFDSPGKLVKMWENEVKDFNPN
IEWSKNNLKKTGHYTQMVWAKTKEIGCGSVKYVKDEWYTHYLVCNYGP
SGNFRNEKLYEKK

### Additional Vespula Sequences (NCBI Entrez Accession):

549193; 549192; 549191; 549190; 549189; 117414; 126761; 69576; 625255; 627189; 627188; 627187; 482382; 112561; 627186; 627185; 1923233; 897645; 897647; 745570; 225764; 162551.

Tree allergen sequences (mainly birch)
sequences:
114922 Bet v 1
MGVFNYETETTSVIPAARLFKAFILDGDNLFPKVAPQAISSVENIEGNGG
PGTIKKISFPEGFPFKYVKDRVDEVDHTNFKYNYSVIEGGPIGDTLEKIS
NEIKIVATPDGGSILKISNKYHTKGDHEVKAEQVKASKEMGETLLRAVES
YLLAHSDAYN
130975 Bet v 2
MSWQTYVDEHLMCDIDGQASNSLASAIVGHDGSVWAQSSSFPQFKPQEIT
GIMKDFEEPGHLAPTGLHLGGIKYMVIQGEAGAVIRGKKGSGGITIKKTG
QALVFGIYEEPVTPGQCNMVVERLGDYLIDQGL
1168696 Bet v 3
MPCSTEAMEKAGHGHASTPRKRSLSNSSFRLRSESLNTLRLRRIFDLFDK
NSDGIITVDELSRALNLLGLETDLSELESTVKSFTREGNIGLQFEDFISL
HQSLNDSYFAYGGEDEDDNEEDMRKSILSQEEADSFGGFKVFDEDGDGYI
SARELQMVLGKLGFSEGSE1DRVEKMIVSVDSNRDGRVDFFEFKDMMRSV
LVRSS
809536 Bet v 4
MADDHPQDKAERERIFKRFDANGDGKISAAELGEALKTLGSITPDEVKHM
MAEIDTDGDGFISFQEFTDFGRANRGLLKDVAKIF
543675 Que a I - Quercus alba = oak trees
(fragment)
GVFTXESQETSVIAPAXLFKALFL
543509 Car b I - Carpinus betulus = hornbeam
trees (fragment)
GVFNYEAETPSVIPAARLFKSYVLDGDKLIPKVAPQAIXK
543491 Aln g I - Alnus glutinosa = alder trees
(fragment)
GVFNYEAETPSVIPAARLFKAFILDGDKLLPKVAPBAVSSVENI
1204056 Rubisco
VQCMQVWPPLGLKKFETLSYLPPLSSEQLAKEVDYLLRKNLIPCLEFELE
HGFVYREHNRSPGYYDGRYWTMWKLPMFGCNDSSQVLKELEECKKAYPS
AFIRIIGFDDK

### Additional Tree Allergen Sequences (NCBI Entrez Accession Number):

131919; 128193; 585564; 1942360; 2554672; 2392209; 2414158; 1321728; 1321726; 1321724; 1321722; 1321720; 1321718; 1321716; 1321714; 1321712; 3015520; 2935416; 464576; 1705843; 1168701; 1168710; 1168709; 1168708; 1168707; 1168706; 1168705; 1168704; 1168703; 1168702; 1842188; 2564228; 2564226; 2564224; 2564222; 2564220; 2051993; 1813891; 1536889; 534910; 534900; 534898; 1340000; 1339998; 2149808; 66207; 2129477; 1076249; 1076247; 629480; 481805; 81443; 1361968; 1361967; 1361966; 1361965; 1361964; 1361963; 1361962; 1361961; 1361960; 1361959; 320546; 629483; 629482; 629481; 541804; 320545; 81444; 541814; 629484; 474911; 452742; 1834387; 298737; 298736; 1584322; 1584321; 584320; 1542873; 1542871; 1542869; 1542867; 1542865; 1542863; 1542861; 1542859; 1542857; 1483232; 1483230; 1483228; 558561; 551640; 488605; 452746; 452744; 452740; 452738; 452736; 452734; 452732; 452730; 452728; 450885; 17938; 17927; 17925; 17921; 297538; 510951; 289331; 289329; 166953.

Peanut
Peanut sequences
1168391 Ara h 1
MRGRVSPLMLLLGILVLASVSATHAKSSPYQKKTENPCAQRCLQSCQQEP
DDLKQKACESRCTKLEYDPRCVYDPRGHTGTTNQRSPPGERTRGRQPGD
YDDDRRQPRREEGGRWGPAGPREREREEDWRQPREDWRRPSHQQPRKIR
PEGREGEQEWGTPGSHVREETSRNNPFYFPSRRFSTRYGNQNGRIRVLQR
FDQRSRQFQNLQNHRIVQIEAKPNTLVLPKHADADNILVIQQGQATVTVA
NGNNRKSFNLDEGHALRIPSGFISYILNRHDNQNLRVAKISMPVNTPGQF
EDFFPASSRDQSSYLQGFSRNTLEAAFNAEFNEIRRVLLEENAGGEQEER
GQRRWSTRSSENNEGVIVKVSKEHVEELTKHAKSVSKKGSEEEGDITNPI
NLREGEPDLSNNFGKLFEVKPDKKNPQLQDLDMMLTCVEIKEGALMLPHF
NSKAMVIVVVNKGTGNLELVAVRKEQQQRGRREEEEDEDEEEEGSNREVR
RYTARLKEGDVFIMPAAHPVAINASSELHLLGFGINAENNHRIFLAGDKD
NVIDQIEKQAKDLAFPGSGEQVEKLIKNQKESHFVSARPQSQSQSPSSPE
KESPEKEDQEEENQGGKGPLLSILKAFN
Ragweed
Ambrosia sequences
113478 Amb a 1
MGIKHCCYILYFTLALVTLLQPVRSAEDLQQILPSANETRSLTTCGTYNI
IDGCWRGKADWAENRKALADCAQGFAKGTIGGKDGDIYTVTSELDDDVAN
PKEGTLRFGAAQNRPLWIIFARDMVIRLDRELAINNDKTIDGRGAKVEII
NAGFAIYNVKNIIIHNIIMHDIVVNPGGLIKSHDGPPVPRKGSDGDAIGI
SGGSQIWIDHCSLSKAVDGLIDAKHGSTHFTVSNCLFTQHQYLLLFWDFD
ERGMLCTVAFNKFTDNVDQRMPNLRHGFVQVVNYERWGSYALGGSAGPTI
LSQGNRFLASDEKKEVVGRYGESAMSESINWNWRSYMDVFENGAIFVPSG
VDPVLTPEQNAGMIPAEPGEAVLRLTSSAGVLSCQPGAPC
113479 Amb a 2
MGIKHCCYILYFTLALVTLVQAGRLGEEVDILPSPNDTRRSLQGCEAHNI
IDKCWRCKPDWAENRQALGNCAQGFGKATHGGKWGDIYMVTSDQDDDVV
NPKEGTLRFGATQDRPLWIIFQRDMIIYLQQEMVVTSDKTIDGRGAKVEL
VYGGITLMNVKNVIIHNIDIHDVRVLPGGRIKSNGGPAIPRHQSDGDAIH
VTGSSDIWIDHCTLSKSFDGLVDVNWGSTGVTISNCKFTHHEKAVLLGAS
DTHFQDLKMHVTLAYNIFTNTVHERMPRCRFGFFQIVNNFYDRWDKYAIG
GSSNPTILSQGNKFVAPDFIYKKNVCLRTGAQEPEWMTWNWRTQNDVLEN
GAIFVASGSDPVLTAEQNAGMMQAEPGDMVPQLTMNAGVLTCSPGAPC
113477 Amb a 1.3
MGIKQCCYILYFTLALVALLQPVRSAEGVGEILPSVNETRSLQACEALNI
IDKCWRGKADWENNRQALADCAQGFAKGTYGGKWGDVYTVTSNLDDDV
ANPKEGTLRFAAAQNRPLWIIFKNDMVINLNQELVVNSDKTIDGRGVKVE
IINGGLTLMNVKNIIIHNINIHDVKVLPGGMIKSNDGPPILRQASDGDTI
NVAGSSQIWIDHCSLSKSFDGLVDVTLGSTHVTISNCKFTQQSKAILLGA
DDTHVQDKGMLATVAFNMFTDNVDQRMPRCRFGFFQVVNNNYDRWGTYAI
GGSSAPTILCQGNRFLAPDDQIKKNVLARTGTGAAESMAWNWRSDKDLLE
NGAIFVTSGSDPVLTPVQSAGMIPAEPGEAAIKLTSSAGVFSCHPGAPC
113476 Amb a 1.2
MGIKHCCYILYFTLALVTLLQPVRSAEDVEEFLPSANETRRSLKACEAHN
IIDKCWRCKADWANNRQALADCAQGFAKGTYGGKHGDVYTVTSDKDDD
VANPKEGTLRFAAAQNRPLWIIFKRNMVIHLNQELVVNSDKTIDGRGVKV
NIVNAGLTLMNVKNIIIHNINIHDIKVCPGGMIKSNDGPPILRQQSDGDA
INVAGSSQIWIDHCSLSKASDGLLDITLGSSHVTVSNCKFTQHQFVLLLG
ADDTHYQDKGMLATVAFNMFTDHVDQRMPRCRFGFFQVVNNNYDRWGTYA
IGGSSAPTILSQGNRFFAPDDIIKKNVLARTGTGNAESMSWNWRTDRDLL
ENGAIFLPSGSDPVLTPEQKAGMIPAEPGEAVLRLTSSAGVLSCHQGAPC
113475 Amb a 1.1
MGIKHCCYILYFTLALVTLLQPVRSAEDLQEILPVNETRRLTTSGAYNII
DGCWRGKADWAENRKALADCAQGFGKGTVGGKDGDIYTVTSELDDDVAN
PKEGTLRFGAAQNRPLWIIFERDMVIRLDKEMVVNSDKTIDGRGAKVEII
NAGFTLNGVKNVIIHNINMHDVKVNPGGLIKSNDGPAAPRAGSDGDAISI
SGSSQIWIDHCSLSKSVDGLVDAKLGTTRLTVSNSLFTQHQFVLLFGAGD
ENIEDRGMLATVAFNTFTDNVDQRMPRCRHGFFQVVNNNYDKWGSYAIGG
SASPTILSQGNRFCAPDERSKKNVLGRHGEAAAESMKWNWRTNKDVLENG
AIFVASGVDPVLTPEQSAGMIPAEPGESALSLTSSAGVLSCQPGAPC
Cedar sequences
493634 Cry j IB precursor
MDSPCLVALLVFSFVIGSCFSDNPIDSCWRGDSNWAQNRMKLADCAVGF
GSSTMGGKGGDLYTVTNSDDDPVNPPGTLRYGATRDRPLWIIFSGNMNIK
LKMPMYIAGYKTFDGRGAQVYIGNGGPCVFIKRVSNVIIHGLYLYGCSTS
VLGNVLINESFGVEPVHPQDGDALTLRTATNIWIDHNSFSNSSDGLVDVT
LTSTGVTISNNLFFNHHKVMSLGHDDAYSDDKSMKVTVAFNQFGPNCGQR
MPRARYGLVHVANNNYDPWTIYAIGGSSNPTILSEGNSFTAPNESYKKQV
TIRIGCKTSSSCSNWVWQSTQDVFYNGAYFVSSGKYEGGNIYTKKEAFNV
ENGNATPHLTQNAGVLTCSLSKRC
493632 Cry j IA precursor
MDSPCLVALLVLSFVIGSCFSDNPIDSCWRGDSNWAQNRMKLADCAVGF
GSSTMGGKGGDLYTVTNSDDDPVNPAPGTLRYGATRDRPLWIIFSGNMNI
KLKMPMYIAGYKTFDGRGAQVYIGNGGPCVFIKRVSNVIIHGLHLYGCST
SVLGNVLINESFGVEPVHPQDGDALTLRTATNIWIDHNSFSNSSDGLVDV
TLSSTGVTISNNLFFNHHKVMLLGHDDAYSDDKSMKVTVAFNQFGPNCGQ
RMPRARYGLVHVANNNYDPWTIYAIGGSSNPTILSEGNSFTAPNESYKKQ
VTIRIGCKTSSSCSNWVWQSTQDVFYNGAYFVSSGKYEGGNIYTKKEAFN
VENGNATPQLTKNAGVLTCSLSKRC
1076242 Cry j II precursor - Japanese cedar
MAMKLIAPMAFLAMQLIIMAAAEDQSAQIMLDSVVEKYLRSNRSLRKVE
HSRHDAINIFNVEKYGAVGDGKHDCTEAFSTAWQAACKNPSAMLLVPGS
KKFVVNNLFFNGPCQPHFTFKVDGIIAAYQNPASWKNNRIWLQFAKLTGF
TLMGKGVIDGQGKQWWAGQCKWVNGREICNDRDRPTAIKFDFSTGLIIQ
GLKLMNSPEFHLVFGNCEGVKIIGISITAPRDSPNTDGIDIFASKNFHLQ
KNTIGTGDDCVAIGTGSSNIVIEDLICGPGHGISIGSLGRENSRAEVSYV
HVNGAKFIDTQNGLRIKTWQGGSGMASHIIYENVEMINSENPILINQFYC
TSASACQNQRSAVQIQDVTYKNIRGTSATAAAIQLKCSDSMPCKDIKLSD
ISLKLTSGKIASCLNDNANGYFSGHVIPACKNLSPSAKRKESKSHKHPKT
VMVENMRAYDKGNRTRILLGSRPPNCTNKCHGCSPCKAKLVIVHRIMPQE
YYPQRWICSCHGKIYHP
1076241 Cry j II protein - Japanese cedar
MAMKFIAPMAFVAMQLIIMAAAEDQSAQIMLDSDIEQYLRSNRSLRKVEH
SRHDAINIFNVEKYGAVGDGKHDCTEAFSTAWQAACKKPSAMLLVPGNK
KFVVNNLFFNGPCQPHFTFKVDGIIAAYQNPASWKNNRIWLQFAKLTGFT
LMGKGVIDGQGKQWWAGQCKWVNGREICNDRDRPTAIKFDFSTGLIIQG
LKLMNSPEFHLVEGNCEGVKIIGISITAPRDSPNTDGIDIFASKNFHLQK
NTIGTGDDCVAIGTGSSNIVIEDLICGPGHGISIGSLGRENSRAEVSYVH
VNGAKFIDTQNGLRIKTWQGGSGMASHIIYENVEMINSENPILINQFYCT
SASACQNQRSAVQIQDVTYKNIRGTSATAAAIQLKCSDSMPCKDIKLSDI
SLKLTSGKIASCLNDNANGYFSGHVIPACKNLSPSAKRKESKSHKHPKTV
MVKNMGAYDKGNRTRILLGSRPPNCTNKCHGCSPCKAKLVIVHRIMPQEY
YPQRWMCSRHGKIYHP
541803 Cry j I precursor - Japanese cedar
MDSPCLVALLVLSFVIGSCFSDNPIDSCWRGDSNWAQNRMKLADCAVGF
GSSTMGGKGGDLYTVTNSDDDPVNPPGTLRYGATRDRPLWIIFSGNMNIK
LKMPMYIAGYKTFDGRGAQVYIGNGGPCVFIKRVSNVIIHGLHLYGCSTS
VLGNVLINESFGVEPVHPQDGDALTLRTATNIWIDHNSFSNSSDGLVDVT
LSSTGVTISNNLFFNHHKVMLLGHDDAYSDDKSMKVTVAFNQFGPNCGQR
MPRARYGLVHVANNNYDPWTIYAIGGSSNPTILSEGNSFTAPNESYKKQV
TIRIGCKTSSSCSNWVWQSTQDVFYNGAYFVSSGKYEGGNIYTKKEAFNV
ENGNATPQLTKNAGVLTCSLSKRC
541802 Cry j I precursor- Japanese cedar
MDSPCLVALLVFSFVIGSCFSDNPIDSCWRGDSNWAQNRMKLADCAVGF
GSSTMGGKGGDLYTVTNSDDDPVNPAPGTLRYGATRDRPLWIIFSGNMNI
KLKMPMYIAGYKTFDGRGAQVYIGNGGPCVFIKRVSNVIIHGLYLYGCST
SVLGNVLINESFGVEPVHPQDGDALTLRTATNIWIDHNSFSNSSDGLVDV
TLTSTGVTISNNLFFNHHKVMSLGHDDAYSDDKSMKVTVAFNQFGPNCGQ
RMPRARYGLVHVANNNYDPWTIYAIGGSSNPTILSEGNSFTAPNESYKKQ
VTIRIGCKTSSSCSNWVWQSTQDVFYNGAYFVSSGKYEGGNIYTKKEAFN
VENGNATPHLTQNAGVLTCSLSKRC
Dog
Canis sequences:
Can f 1
MKTLLLTIGFSLIAILQAQDTPALGKDTVAVSGKWYLKAMTADQEVPEKP
DSVTPMILKAQKGGNLEAKITMLTNGQCQNITVVLHKTSEPGKYTAYEGQ
RVVFIQPSPVRDHYILYCEGELHGRQIRMAKLLGRDPEQSQEALEDFREF
SRAKGLNQEILELAQSETCSPGGQ
Serum albumin fragment
EAYKSEIAHRYNDLGEEHFRGLVL
Serum albumin fragment
LSSAKERFKCASLQKFGDRAFKAWSVARLSQRFPKADFAEISKVVTDLTK
VHKECCHGDLLECADDRADLAKYMCENQDSISTKLKECCDKPVLEKSQC
LAEVERDELPGDLPSLAADFVEDKEVCKNYQEAKDVFLGTFLYEYSRRHP
EYSVSLLLRLAKEYEATLEKCCATDDPPTCYAKVLDEFKPLVDEPQNLVK
TNCELFEKLGEYGFQNALLVRYTKKAPQVSTPTLVVEVSRKLGKVGTKCC
KKPESERMSCADDFLS
Can f 2
MQLLLLTVGLALICGLQAQEGNHEEPQGGLEELSGRWHSVALASNKSDLI
KPWGHFRVFIHSMSAKDGNLHGDILIPQDGQCEKVSLTAFKTATSNKFDL
EYWGHNDLYLAEVDPKSYLILYMINQYNDDTSLVAHLMVRDLSRQQDFL
PAFESVCEDIGLHKDQIVVLSDDDRCQGSRD

### Additional Dog Allergen Protein (NCBI Entrez Accession):

1731859

Horse
Equus sequences:
1575778 Equ cl
MKLLLLCLGLILVCAQQEENSDVAIRNFDISKISGEWYSIFLASDVKEKI
EENGSMRVFVDVIRALDNSSLYAEYQTKVNGECTEFPMVFDKTEEDGVYS
LNYDGYNVFRISEFENDEHIILYLVNFDKDRPFQLFEFYAREPDVSPEIK
EEFVKIVQKRGIVKENIIDLTKIDRCFQLRGNGVAQA
3121755 Equ c 2
SQXPQSETDYSQLSGEWNTIYGAASNIXK
Euroglyphus (mite)
Euroglyphus sequences:
Eur m 1 (variant)
TYACSINSVSLPSELDLRSLRTVTPIRMQGGCGSCWAFSGVASTESAYLA
YRNMSLDLAEQELVDCASQNGCHGDTIPRGIEYIQQNGVVQEHYYPYVAR
EQSCHRPNAQRYGLKNYCQISPPDSNKIRQALTQTHTAVAVIIGIKDLNA
FRHYDGRTIMQHDNGYQPNYHAVNIVGYGNTQGVDYWIVRNSWDTTWGD
NGYGYFAANINL
Eur m 1 (variant)
TYACSINSVSLPSELDLRSLRTVTPIRMQGGCGSCWAFSGVASTESAYLA
YRNMSLDLAEQELVDCASQNGCHGDTIPRGIEYIQQNGVVQEHYYPYVAR
EQSCHRPNAQRYGLKNYCQISPPDSNKIRQALTQTHTAVAVIIGIKDLNA
FRHYDGRTIMQHDNGYQPNYHAVNIVGYGNTQGVDYWIVRNSWDTTWGD
NGYGYFAANINL
Eur m 1 (variant)
ETNACSINGNAPAEIDLRQMRTVTPIRMQGGCGSCWAFSGVAATESAYLA
YRNQSLDLAEQELVDCASQHGCHGDTDPRGIEYIQHNGVVQESYYRYVAR
EQSCRRPNAQRFGISNYCQIYPPNANKIREALAQTHSAIAVIIGIKDLDA
FRHYDGRTIIQRDNGYQPNYHAVNIVGYSNAQGVDYWIVRNSWDTNWGDN
GYGYFAANIDL
Eur m 1 (variant)
ETSACRINSVNVPSELDLRSLRTVTPIRMQGGCGSCWAFSGVAATESAYL
AYRNTSLDLSEQELVDCASQHGCHGDTIPRGIEYIQQNGVVEERSYPYVA
REQQCRRPNSQHYGISNYCQIYPPDVKQIREALTQTHTAIAVIIGIKDLR
AFQHYDGRTIIQHDNGYQPNYHAVNIVGYGSTQGVDYWIVRNSWDTTWGD
SGYGYFQAGNNL
Poa (grass) sequences
113562 POLLEN ALLERGEN POA P 9
MAVQKYTVALFLVALVVGPAASYAADLSYGAPATPAAPAAGYTPAAPA
GAAPKATTDEQKMIEKINVGFKAAVAAAGGVPAANKYKTFVATFGAASN
KAFAEALSTEPKGAAVDSSKAALTSKLDAAYKLAYKSAEGATPEAKYDD
YVATLSEALRIIAGTLEVHGVKPAAEEVKATPAGELQVIDKVDAAFKVAA
TAANAAPANDKFTVFEAAFNDAIKASTGGAYQSYKFIPALEAAVKQSYAA
TVATAPAVKYTVFETALKKAITAMSQAQKAAKPAAAATGTATAAVGAA
TGAATAAAGGYKV
113561 POA P 9
MAVHQYTVALFLAVALVAGPAASYAADVGYGAPATLATPATPAAPAAG
YTPAAPAGAAPKATTDEQKLIEKINAGFKAAVAAAAGVPAVDKYKTFVA
TFGTASNKAFAEALSTEPKGAAAASSNAVLTSKLDAAYKLAYKSAEGATP
EAKYDAYVATLSEALRIIAGTLEVHAVKPAGEEVKAIPAGELQVIDKVDA
AFKVAATAANAAPANDKFTVFEAAFNDAIKASTGGAYQSYKFIPALEAAV
KQSYAATVATAPAVKYTVFETALKKAITAMSQAQKAAKPAAAVTATAT
GAVGAATGAVGAATGAATAAAGGYKTGAATPTAGGYKV
113560 POA P 9
MDKANGAYKTALKAASAVAPAEKFPVFQATFDKNLKEGLSGPDAVGFA
KKLDAFIQTSYLSTKAAEPKEKFDLFVLSLTEVLRFMAGAVKAPPASKFP
AKPAPKVAAYTPAAPAGAAPKATTDEQKLIEKINVGFKAAVAAAAGVPAA
SKYKTFVATFGAASNKAFAEALSTEPKGAAVASSKAVLTSKLDAAYKLA
YKSAEGATPEAKYDAYVATLSEALRIIAGTLEVHGVKPAABEVKAIPAGE
LQVIDKVDAAFKVAATAANAAPANDKFTVFEAAFNDAIKASTGGAYQSY
KFIPALEAAVKQSYAATVATAPAVKYTVFETALKKAITAMSQAQKAAKP
AAAVTGTATSAVGAATGAATAAAGGYKV
Cockroach sequences
2833325 Cr p1
MKTALVFAAVVAFVAARFPDHKDYKQLADKQFLAKQRDVLRLFHRVHQ
HNILNDQVEVGIPMTSKQTSATTVPPSGEAVHGVLQEGHARPRGEPFSVN
YEKHREQAIMLYDLLYFANDYDTFYKTACWARDRVNEGMFMYSFSIAVF
HRDDMQGVMLPPPYEVYPYLFVDHDVIHMAQKYWMKNAGSGEHHSHVI
PVNFTLRTQDHLLAYFTSDVNLNAFNTYYRYYYPSWYNTTLYGHNIDRR
GEQFYYTYKQIYARYFLERLSNDLPDVYPFYYSKPVKSAYNPNLRYHNGE
EMPVRPSNMYVTNFDLYYIADIKNYEKRVEDAIDFGYAFDEHMKPHSLY
HDVHGMEYLADMIEGNMDSPNFYFYGSIYHMYHSMIGHIVDPYHKMGL
APSLEHPETVLRDPVFYQLWKRVDHLFQKYKNRLPRYTHDELAFEGVKV
ENVDVGKLYTYFEQYDMSLDMAVYVNNVDQISNVDVQLAVRLNHKPFT
YNIEVSSDKAQDVYVAVFLGPKYDYLGREYDLNDRRHYFVEMDRFPYHV
GAGKTVIERNSHDSNIIAPERDSYRTFYKKVQEAYEGKSQYYVDKGHNYC
GYPENLLIPKGKKGGQAYTFYVIVTPYVKQDEHDFEPYNYKAFSYCGVGS
ERKYPDNKPLGYPFDRKIYSNDFYTPNMYFKDVIIFHKKYDEVGVQGH
2231297 Cr p2
INEIHSIIGLPPFVPPSRRHARRGVGINGLIDDVIAILPVDELKALFQEK
LETSPDFKALYDAIRSPEFQSIISTLNAMQRSEHHQNLRDKGVDVDHFIQ
LIRALFGLSRAARNLQDDLNDFLHSLEPISPRHRHGLPRQRRRSARVSAY
LHADDFHKIITTIEALPEFANFYNFLKEHGLDVVDYINEIHSIIGLPPFV
PPSRRHARRGVGINGLIDDVIAILPVDELKALFQEKLETSPDFKALYDAI
RSPEFQSIISTLNAMPEYQELLQNLRDKGVDVDHFIRVDQGTLRTLSSGQ
RNLQDDLNDFLALIPTDQILAIAMDYLANDAEVQELVAYLQSDDFHKIIT
TIEALPEFANFYNFLKEHGLDVVDYINELHSIIGLPPFVPPSQRHARRGV
GINGLIDDVIAILPVDELKALFQEKLETSPDFKALYDAIDLRSSRA
1703445 Bla g 2
MIGLKLVTVLFAVATITHAAELQRVPLYKLVHVFINTQYAGITKIGNQNF
LTVFDSTSCNVVVASQECVGGACVCPNLQKYEKLKPKYISDGNVQVKFFD
TGSAVGRGIEDSLTISNLTTSQQDIVLADELSQEVCILSADVVVGIAAPG
CPNALKGKTVLENFVEENLIAPVFSLHHARFQDGEHFGEIIFGGSDWKYV
DGEFTYVPLVGDDSWKFRLDGVKIGDTTVAPAGTQAIIDTSKAIIVGPKA
YVNPINEAIGCVVEKTTTRRICKLDCSKIPSLPDVTFVINGRNFNISSQY
YIQQNGNLCYSGFQPCGHSDHFFIGDFFVDHYYSEFNWENKTMGFGRSVE
SV
1705483 Bla g 4
AVLALCATDTLANEDCFRHESLVPNLDYERFRGSWIIAAGTSEALTQYKC
WIDRFSYDDALVSKYTDSQGKNRTTIRGRTKFEGNKFTIDYNDKGKAFSA
PYSVLATDYENYAIVEGCPAAANGHVIYVQLRFSVRRFHPKLGDKEMIQH
YTLDQVNQHKKAIEEDLKHFNLKYEDLHSTCH
2326190 Bla g 5
YKLTYCPVKALGEPIRFLLSYGEKDFEDYRFQEGDWPNLKPSMPFGKTPV
LEIDGKQTHQSVAISRYLGKQFGLSGKDDWENLEIDMIVDTISDFRAAIA
NYHYDADENSKQKKWDPLKKETIPYYTKKFDEVVKANGGYLAAGKLTWA
DFYFVAILDYLNHMAKEDLVANQPNLKALREKVLGLPAIKAWVAKRPPT
DL

### Additional Cockroach Sequences (NCBI Entrez Accession Numbers):

2580504; 1580797; 1580794; 1362590; 544619; 544618; 1531589; 1580792; 1166573; 1176397; 2897849.

### Allergen (General) Sequences:

### NCBI Accession Numbers

2739154; 3719257; 3703107; 3687326; 3643813; 3087805; 1864024; 1493836; 1480457; 2598976; 2598974; 1575778; 763532; 746485; 163827; 163823; 3080761; 163825; 3608493; 3581965; 2253610; 2231297; 2897849; 3409499; 3409498; 3409497; 3409496; 3409495; 3409494; 3409493; 3409492; 3409491; 3409490; 3409489; 3409488; 3409487; 3409486; 3409485; 3409484; 3409483; 3409482; 3409481; 3409480; 3409479; 3409478; 3409477; 3409476; 3409475; 3409474; 3409473; 3409472; 3409471; 3409470; 3409469; 3409468; 3409467; 3409466; 3409465; 3409464; 3409463; 3409462; 3409461; 3409460; 3409459; 3409458; 3409457; 3409456; 3318885; 3396070; 3367732; 1916805; 3337403; 2851457; 2851456; 1351295; 549187; 136467; 1173367; 2499810; 2498582; 2498581; 1346478; 1171009; 126608; 114091; 2506771; 1706660; 1169665; 1169531; 232086; 416898; 114922; 2497701; 1703232; 1703233; 1703233; 1703232; 3287877; 3122132; 3182907; 3121758; 3121756; 3121755; 3121746; 3121745; 3319925; 3319923; 3319921; 3319651; 3318789; 3318779; 3309647; 3309047; 3309045; 3309043; 3309041; 3309039; 3288200; 3288068; 2924494; 3256212; 3256210; 3243234; 3210053; 3210052; 3210051; 3210050; 3210049; 3210048; 3210047; 3210046; 3210045; 3210044; 3210043; 3210042; 3210041; 3210040; 3210039; 3210038; 3210037; 3210036; 3210035; 3210034; 3210033; 3210032; 3210031; 3210030; 3210029; 3210028; 3210027; 3210026; 3210025; 3210024; 3210023; 3210022; 3210021; 3210020; 3210019; 3210018; 3210017; 3210016; 3210015; 3210014; 3210013; 3210012; 3210011; 3210010; 3210009; 3210008; 3210007; 3210006; 3210005; 3210004; 3210003; 3210002; 3210001; 3210000; 3209999; 3201547; 2781152; 2392605; 2392604; 2781014; 1942360; 2554672; 2392209; 3114481; 3114480; 2981657; 3183706; 3152922; 3135503; 3135501; 3135499; 3135497; 2414158; 1321733; 1321731; 1321728; 1321726; 1321724; 1321722; 1321720; 1321718; 1321716; 1321714; 1321712; 3095075; 3062795; 3062793; 3062791; 2266625; 2266623; 2182106; 3044216; 2154736; 3021324; 3004467; 3005841; 3005839; 3004485; 3004473; 3004471; 3004469; 3004465; 2440053; 1805730; 2970629; 2959898; 2935527; 2935416; 809536; 730091; 585279; 584968; 2498195; 2833325; 2498604; 2498317; 2498299; 2493414; 2498586; 2498585; 2498576; 2497749; 2493446; 2493445; 1513216; 729944; 2498099; 548449; 465054; 465053; 465052; 548671; 548670; 548660; 548658; 548657; 2832430; 232084; 2500822; 2498118; 2498119; 2498119; 2498118; 1708296; 1708793; 416607; 416608; 416608; 416607; 2499791; 2498580; 2498579; 2498578; 2498577; 2497750; 1705483; 1703445; 1709542; 1709545; 1710589; 1352699; 1346568; 1346323; 1346322; 2507248; 11352240; 1352239; 1352237; 1352229; 1351935; 1350779; 1346806; 1346804; 1346803; 1170095; 1168701; 1352506; 1171011; 1171008; 1171005; 1171004; 1171002; 1171001; 1168710; 1168709; 1168708; 1168707; 1168706; 1168705; 1168704; 1168703; 1168702; 1168696; 1168391; 1168390; 1168348; 1173075; 1173074; 1173071; 1169290; 1168970; 1168402; 729764; 729320; 729979; 729970; 729315; 730050; 730049; 730048; 549194; 549193; 549192; 549191; 549190; 549189; 549188; 549185; 549184; 549183; 549182; 549181; 549180; 549179; 464471; 585290; 416731; 1169666; 113478; 113479; 113477; 113476; 113475; 130975; 119656; 113562; 113561; 113560; 416610; 126387; 126386; 126385; 132270; 416611; 416612; 416612; 416611; 730035; 127205; 1352238; 125887; 549186; 137395; 730036; 133174; 114090; 131112; 126949; 129293; 124757; 129501; 416636; 2801531; 2796177; 2796175; 2677826; 2735118; 2735116; 2735114; 2735112; 2735110; 2735108; 2735106; 2735104; 2735102; 2735100; 2735098; 2735096; 2707295; 2154730; 2154728; 1684720; 2580504; 2465137; 2465135; 2465133; 2465131; 2465129; 2465127; 2564228; 2564226; 2564224; 2564222; 2564220; 2051993; 1313972; 1313970; 1313968; 1313966; 2443824; 2488684; 2488683; 2488682; 2488681; 2488680; 2488679; 2488678; 2326190; 2464905; 2415702; 2415700; 2415698; 2398759; 2398757; 2353266; 2338288; 1167836; 414703; 2276458; 1684718; 2293571; 1580797; 1580794; 2245508; 2245060; 1261972; 2190552; 1881574; 511953; 1532058; 1532056; 1532054; 1359436; 666007; 487661; 217308; 1731859; 217306; 217304; 1545803; 1514943; 577696; 516728; 506858; 493634; 493632; 2154734; 2154732; 543659; 1086046; 1086045; 2147643; 2147642; 1086003; 1086002; 1086001; 543675; 543623; 543509; 543491; 1364099; 2147108; 2147107; 1364001; 1085628; 631913; 631912; 631911; 2147092; 477301; 543482; 345521; 542131; 542130; 542129; 100636; 2146809; 480443; 2114497; 2144915; 72355; 71728; 319828; 1082946; 1082945; 1082944; 539716; 539715; 423193; 423192; 423191; 423190; 1079187; 627190; 627189; 627188; 627187; 482382; 1362656; 627186; 627185; 627182; 482381; 85299; 85298; 2133756; 2133755; 1079186; 627181; 321044; 321043; 112559; 112558; 1362590; 2133564; 1085122; 1078971; 627144; 627143; 627142; 627141; 280576; 102835; 102834; 102833; 102832; 84703; 84702; 84700; 84699; 84698; 84696; 477888; 477505; 102575; 402572; 478272; 2130094; 629813; 629812; 542172; 542168; 542167; 481432; 320620; 280414; 626029; 542132; 320615; 320614; 100638; 100637; 100635; 82449; 320611; 320610; 280409; 320607; 320606; 539051; 539050; 539049; 539048; 322803; 280407; 100501; 100498; 100497; 100496; 1362137; 1362136; 1362135; 1362134; 1362133; 1362132; 1362131; 1362130; 1362129; 1362128; 100478; 2129891; 1076531; 1362049; 1076486; 2129817; 2129816; 2129815; 2129814; 2129813; 2129812; 2129805; 2129804; 2129802; 2129801; 2129800; 2129799; 479902; 479901; 2129477; 1076247; 629480; 1076242; 1076241; 541803; 541802; 280372; 280371; 1361968; 1361967; 1361966; 1361965; 1361964; 1361963; 1361962; 1361961; 1361960; 1361959; 320546; 2119763; 543622; 541804; 478825; 478824; 478823; 421788; 320545; 81444; 626037; 626028; 539056; 483123; 481398; 481397; 100733; 100732; 100639; 625532; 1083651; 322674; 322673; 81719; 81718; 2118430; 2118429; 2118428; 2118427; 419801; 419800; 419799; 419798; 282991; 100691; 322995; 322994; 101824; 626077; 414553; 398830; 1311457; 1916292; 1911819; 1911818; 1911659; 1911582; 467629; 467627; 467619; 467617; 915347; 1871507; 1322185; 1322183; 897645; 897647; 1850544; 1850542; 1850540; 288917; 452742; 1842045; 1839305; 1836011; 1836010; 1829900; 1829899; 1829898; 1829897; 1829896; 1829895; 1829894; 1825459; 1808987; 159653; 1773369; 1769849; 1769847; 608690; 1040877; 1040875; 1438761; 1311513; 1311512; 1311511; 1311510; 1311509; 1311689; 1246120; 1246119; 1246118; 1246117; 1246116; 1478293; 1478292; 1311642; 1174278; 1174276; 1086972; 1086974; 1086976; 1086978; 1086978; 1086976; 1086974; 1086972; 999009; 999356; 999355; 994866; 994865; 913758; 913757; 913756; 913285; 913283; 926885; 807138; 632782; 601807; 546852; 633938; 544619; 544618; 453094; 451275; 451274; 407610; 407609; 404371; 409328; 299551; 299550; 264742; 261407; 255657; 250902; 250525; 1613674; 1613673; 1613672; 1613671; 1613670; 1613304; 1613303; 1613302; 1613240; 1613239; 1613238; 1612181; 1612180; 1612179; 1612178; 1612177; 1612176; 1612175; 1612174; 1612173; 1612172; 1612171; 1612170; 1612169; 1612168; 1612167; 1612166; 1612165; 1612164; 1612163; 1612162; 1612161; 1612160; 1612159; 1612158; 1612157; 1612156; 1612155; 1612154; 1612153; 1612152; 1612151; 1612150; 1612149; 1612148; 1612147; 1612146; 1612145; 1612144; 1612143; 1612142; 1612141; 1612140; 1612139; 1093120; 447712; 447711; 447710; 1587177; 158542; 1582223; 1582222; 1531589; 1580792; 886215; 1545897; 1545895; 1545893; 1545891; 1545889; 1545887; 1545885; 1545883; 1545881; 1545879; 1545877; 1545875; 166486; 1498496; 1460058; 972513; 1009442; 1009440; 1009438; 1009436; 1009434; 7413; 1421808; 551228; 452606; 32905; 1377859; 1364213; 1364212; 395407; 22690; 22688; 22686; 22684; 488605; 17680; 1052817; 1008445; 1008443; 992612; 706811; 886683; 747852; 939932; 19003; 1247377; 1247375; 1247373; 862307; 312284; 999462; 999460; 999458; 587450; 763064; 886209; 1176397; 1173557; 902012; 997915; 997914; 997913; 997912; 997911; 997910; 99790; 997908; 997907; 997906; 997905; 997904; 997903; 997902; 997901; 997900; 997899; 997898; 997897; 997896; 997895; 997894; 997893; 997892; 910984; 910983; 910982; 910981; 511604; 169631; 169629; 169627; 168316; 168314; 607633; 555616; 293902; 485371; 455288; 166447; 166445; 166443; 166435; 162551; 160780; 552080; 156719; 156715; 515957; 515956; 515955; 515954; 515953; 459163; 166953; 386678; 169865.

In other preferred embodiments, the polypeptide antigen is an autoantigen, typically wherein the autoantigen is administered with the secondary composition, that is, is step (2) or (3). Peptide autoantigens useful in the instant methods include, but are not limited to insulin, glutamic acid decarboxylase (64K), PM-1 and carboxypeptidase in diabetes; myelin basic protein in multiple sclerosis; rh factor in erythroblastosis fetalis; acetylcholine receptors in myasthenia gravis; thyroid receptors in Graves' Disease; basement membrane proteins in Good Pasture's syndrome; and thyroid proteins in thyroiditis.

In any regard, once selected, the peptide allergens used herein can be obtained and/or produced using a variety of methods known to those skilled in the art. In particular, the allergens can be isolated directly from native sources, using standard purification techniques. Alternatively, the allergens can be recombinantly produced using expression systems well known in the art and purified using known techniques. The peptide allergens can also be synthesized, based on described amino acid sequences or amino acid sequences derived from the DNA sequence of a nucleic acid molecule of interest, via chemical polymer syntheses such as solid phase peptide synthesis. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides Analysis, Synthesis, Biology, supra, Vol. 1, for classical solution synthesis.

By “T-cell-epitope-containing peptide of an antigen we mean any suitable peptide of an antigen that contains a T-cell epitope. Typically, the peptide of the antigen is a peptide that contains a single T cell epitope but it may contain two or more T cell epitopes. However, as described in more detail below, a mixture of peptides may be given. It is not necessary for each of the peptides in the mixture to be biologically active within the individual provided that one or more of them are recognised by T cells within the individual. For this to occur, the peptide or peptides will need to be presented by (restricted by) an MHC molecule expressed by the individual. Typically, depending on the antigen (allergen) the mixture of peptides may contain 3 or 4 peptides but may contain 5 or 6 or 7 or 8 or 9 or 10 or 11 or 12, or more different peptides.

It can readily be determined whether a peptide contains a T cell epitope of an antigen by using in vitro proliferation assays or cytokine assays, which are known in the art, such as those described in commonly owned WO 99/34826 (PCT/GB99/00080). Preferably, the peptide of the antigen (allergen) administered in step (1), that is, in the primary composition, is one that does not stimulate B cells. Typically, the peptide of the antigen (allergen) is a peptide that has between 6 and 30 amino acids, preferably between 6 and 20 amino acids, more preferably between 7 and 18 amino acids and most preferably between 10 and 14 amino acids. Preferably, the peptide of the antigen (allergen) has the same contiguous amino acid sequence as is found in the relevant portion of the native antigen (allergen).

By “peptide of an antigen” we include the meaning that the peptide is chemically derived from the polypeptide antigen, for example by proteolytic cleavage and we also include the meaning that the peptide is derived in an intellectual sense from the polypeptide antigen, for example by making use of the amino acid sequence of the polypeptide antigen and synthesising peptides based on the sequence.

By “polypeptide antigen from which the peptides in step (1) are derived” we mean a polypeptide antigen that contains the same T cell epitope present in the peptide or peptides administered in step (1), that is, in the primary composition. By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159:3230-3237. This approach involves making pseudopeptides containing changes involving the backbone, and not the orientation of side chains. It has been shown that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion. It is also possible to modify the structure of peptides for such purposes as increasing solubility, enhancing therapeutic or preventive efficacy, or stability (e.g., shelf life ex vivo, and resistance to proteolytic degradation in vivo). A modified peptide can be produced in which the amino acid sequence has been altered, such as by amino acid substitution, deletion, or addition, to modify immunogenicity and/or reduce allergenicity, or to which a component has been added for the same purpose. For example, the amino acid residues essential to T cell epitope function can be determined using known techniques (e.g., substitution of each residue and determination of presence or absence of T cell reactivity). Those residues shown to be essential can be modified (e.g., replaced by another amino acid whose presence is shown to enhance T cell reactivity); as can those which are not required for T cell reactivity (e.g., by being replaced by another amino acid whose incorporation enhances T cell reactivity but does not diminish binding to relevant MHC). Another example of a modification of peptides is substitution of cysteine residues preferably with alanine, or glutamic acid, or alternatively with serine or threonine to minimize dimerization via disulfide linkages. In order to enhance stability and/or reactivity, peptides can also be modified to incorporate one or more polymorphisms in the amino acid sequence of a protein allergen resulting from natural allelic variation. Additionally, D-amino acids, non-natural amino acids or non-amino acid analogues can be substituted or added to produce a modified peptide within the scope of this invention. Furthermore, peptides can be modified using well-known polyethylene glycol (PEG) methods to produce a peptide conjugated with PEG. Modifications of peptides can also include reduction/alkylation (Tarr in: Methods of Protein Microcharacterization, J. E. Silver ed. Humana Press, Clifton, N.J., pp 155-194 (1986)); acylation (Tarr, supra); esterification (Tarr, supra); chemical coupling to an appropriate carrier (Mishell and Shiigi, eds, Selected Methods in Cellular Immunology, W H Freeman, San Francisco, Calif. (1980); U.S. Pat. No. 4,939,239); or mild formalin treatment (Marsh International Archives of Allergy and Applied Immunology 41: 199-215 (1971)).

Peptides may be synthesised by the Fmoc-polyamide mode of solid-phase peptide synthesis as disclosed by Lu et al (1981) J. Org. Chem. 46:3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesised. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers. Reagents for peptide synthesis are generally available from Calbiochem-Novabiochem (UK) Ltd, Nottingham NG7 2QJ, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

Preferably, the polypeptide antigens used herein are those wherein restriction to a MHC Class II molecule possessed by the individual can be demonstrated for the peptide and the peptide is able to induce a late phase response in an individual who possesses the said MHC Class II molecule. Such peptides, compositions of such peptides and methods of identifying such peptides are described in commonly owned International Publication WO 99/34826.

Although the peptide administered in step (1) may be a single peptide, the primary composition may conveniently comprise a plurality of T-cell-epitope containing antigens, for example, a plurality of peptides. The peptides in the composition may or may not be multiple overlapping peptides (MOPs) derived from the same polypeptide antigen. The plurality of peptides may be derived from the whole of the polypeptide antigen and therefore the peptides may span the to whole of the polypeptide chain or chains of the antigen. However, they may be derived from only portions of the polypeptide antigen such that some portions of the polypeptide antigen are not represented in the plurality of peptides (for example, some peptides derived from an antigen may not be very soluble in aqueous solution and so may not be useful, and other peptides may not show restriction to MHC Class II molecules). MOPs or any peptides derived from the antigen and present in the composition can be designed by reference to the amino acid sequence of the polypeptide antigen. Typically, the MOPs are at least seven amino acid residues and more typically between around 10 to 14 amino acid residues in length. It is preferred that the peptides of MOPs have a reduced ability to bind IgE compared to longer peptides containing the same sequence. It is particularly preferred if the peptides or MOPs are substantially incapable of cross-linking IgE on the surface of a mast cell. Typically, when the MOPs overlap, the overlap is around one or two or three or four amino acid residues.

The method may be used to desensitise an individual to a polypeptide allergen. By “desensitising an individual to a polypeptide allergen” is meant inhibition or dampening of allergic tissue reactions induced by allergens in appropriately sensitised individuals. It will be appreciated that whether or not an individual is sensitive to a particular polypeptide allergen can be assessed using well known procedures such as skin prick testing with solutions of allergen extracts, induction of cutaneous late phase responses (LPRs), clinical history, allergen challenge and radio-allergosorbent test (RAST) for measurement of allergen specific IgE, and whether or not a particular individual is one who is expected to benefit from treatment may be determined by the physician based, for example, on such tests.

The method may be used to desensitise an individual to a polypeptide autoantigen. By “desensitising an individual to a polypeptide autoantigen” is meant inhibition or dampening of autologous or allogeneic tissue reactions induced by said antigens in appropriately sensitised individuals. It will be appreciated that whether or not an individual is sensitive to a particular autoantigen can be assessed using well known procedures such as clinical history, in vitro autoantigen challenge of peripheral blood mononuclear cells with the antigen in question, measurement of antigen-specific immunoglobulins (Ig), for example the presence in the serum of clinically significant autoantibodies or antibodies against a graft antigen, or host antigens following allograft; or the presence of visible or measurable inflammation within the tissue. For example, tissue that forms part of an allograft or the tissues of a host having received an allograft, or the central nervous system of an individual with multiple sclerosis, or insulitis in an individual with type 1 diabetes, swollen joints in an individual with rheumatoid arthritis and any other clinical test that may be used to determine autoimmune disease or graft versus host disease or host versus graft disease. Whether or not a particular individual is one who is expected to benefit from treatment may be determined by the physician based, for example, on such tests.

The composition administered in step (2) may comprise a T cell epitope of a peptide administered in step (1) and at least one T cell epitope of a different polypeptide antigen to which the individual is to be desensitised. In a preferred embodiment, the composition is a fusion of a T-cell-epitope containing peptide as defined in step (1) and a different substantially whole antigen to which the individual is to be desensitised. It may be beneficial to use a “non-anaphylactic” version of the antigen when it is an allergen. IgE recognises determinants (epitopes) on the outer surface of a whole protein. Thus, if the peptides or sequences of polypeptide to which you wish to desensitise, are hidden within the chimeric protein, for example, you may effectively sequester them from IgE, thus reducing the chance of IgE-mediated events like anaphylaxis which you may run the risk of if you were simply to administer the whole protein.

By “substantially localised” we include the meaning that upon administration or delivery to a site in the individual which has access to the immune system, at least some of the composition resides at the site for an appreciable length of time such as, for example, 24 to 48 hours and does not diffuse away or disperse to any significant extent. It will be appreciated that some of the composition may diffuse or disperse away from the site of administration or delivery but it remains substantially localised in the context of the invention if sufficient remains present at a particular site to be taken up by antigen presenting cells (APCs) and for recruited T cells to recognise both sets of epitopes in the same APC. Thus, there is preferably a “depot” produced of the composition, or local fixation at the site of administration occurs. The composition may be substantially localised by physical or chemical means. Thus, typically, the composition may be substantially localised by virtue of its molecular weight or by virtue of being present in a slow release formulation or by virtue of being conjugated to a further molecule that leads to substantial localisation at the site of administration. It will be appreciated that for many antigens, the native antigen polypeptide (such as a recombinantly produced antigen) alone is sufficient to be substantially localised when administered. However, it may be beneficial if the antigen is combined with some other agent. Typically, the composition may be a polymer of a peptide or an aggregate or an emulsion thereof. However, in each case, in the composition administered in step (2), the T cell epitope of a peptide administered in step (1) must be accessible to a T cell and the T cell epitope containing portion is kept at the site of administration, at least in sufficient amount and for a sufficient time to have the desired effect as described above.

Depot forming agents include water and mineral oil emulsions where the composition, such as a polypeptide, is solubilised in the aqueous phase prior to emulsification. Conjugates may include conjugates of the composition with a bacterial product which may stimulate uptake by an APC.

The method may be used to desensitise the individual to the polypeptide antigen from which the peptides in step (1) are derived, in which case in step (2) the individual may be administered a composition which contains the T cell epitope of a peptide administered in step (1) and further T cell epitopes of said antigen. Thus, in one embodiment a single composition is administered in step (2) which composition is the substantially whole antigen corresponding to the peptide or peptides in step (1).

In a preferred embodiment, additionally, or alternatively, the method may be used to desensitise the individual to one or more polypeptides other than the polypeptide antigen from which the peptides in step (1) are derived. Thus, in this embodiment, the composition may be one which contains a T cell epitope of a peptide administered in step (1) and which contains a T cell epitope of the one or more polypeptide antigens to which the individual is to be desensitised. Alternatively, in this embodiment, if the composition administered in step (2) does not contain a T cell epitope of the said one or more polypeptide antigens the method comprises the step of (3) further administering to the individual a composition which contains a T cell epitope of the one or more polypeptide antigens to which the individual is to be desensitised.

The composition administered in step (2) can comprises substantially whole antigen from which the peptides in step (1) are derived. In addition, the composition in step (3) can comprises substantially whole antigen to which the individual is to be desensitised.

Without being bound by any theory, we believe that the peptides administered in step (1) are able to generate a state of general hyporesponsiveness which extends not only to the antigen from which the antigen is obtained or derived, but also to other polypeptide antigens. Thus, a type of tolerance is induced by the peptide or peptides administered in step (1). While again not being bound by any theory, we believe that the presence in the composition administered in step (2) of a T cell epitope of a peptide administered in step (1) means that appropriate T cells (i.e., newly induced tolerogenic cells which are specific for it) are drawn into the environment where the composition is substantially localised. When the further composition (e.g., whole antigen to which the individual is to be desensitised in step (3)) is administered, we believe that pathogenic T cells specific for the further antigen are recruited and, at the same local site, the further-antigen-specific cells come under the influence of the tolerogenic cells and are rendered tolerant. The compositions administered in steps (2) and (3) may also contain B cell (antibody) epitopes, particularly when the compositions do not contain allergens. It is known in the art that some T cell epitopes are also B cell epitopes and that some B cell epitopes are T cell epitopes. However, although T cell epitopes are always linear determinants of a polypeptide, B cell epitopes may be non-linear, eg made up of different portions of the polypeptide. Again not being bound by any theory, we believe that antigen-specific B cells are drawn into the environment by the presence in the composition administered in step (2) and (step (3), if carried out) of B-cell (antibody) epitopes of the polypeptide antigen, and that this is advantageous the case of desensitisation to autoimmune proteins or to transplantation allergens (such as in autoimmunity and graft-versus-host or host-versus-graft disease). It is preferred that B cell epitopes are not present, or are hidden, in the compositions administered in steps (2) or (3) in order to try to reduce IgE responses and the possibility of anaphylaxis.

In a second aspect of the invention, we provide a method of desensitising an individual to one or more polypeptide antigens the method comprising: step (1) administering to the individual a composition comprising a T-cell-epitope-containing peptide, or a course of T-cell-epitope-containing peptides, of a first antigen to which the individual has been previously exposed, in order to generate in the individual a state of hyporesponsiveness to the first antigen; then step (2) administering to the individual substantially whole antigen corresponding to the peptide or peptides in step (1); and, if the polypeptide antigen to which the individual is to be desensitised is not the substantially whole first antigen administered in step (2), the method comprises the step of (3) further administering to the individual substantially whole polypeptide antigen or antigens to which the individual is to be desensitised. Preferably, the first antigen is an allergen.

Put in another way, in a second aspect of the invention, a method is provided for desensitising an individual to one or more polypeptide antigens. The method entails, in a first step, administering to the individual a primary composition comprising a peptide antigen containing a T cell epitope, wherein the individual has been previously exposed to the antigen and the administration is carried out in a manner sufficient to generate a hyporesponsive state against the peptide antigen. Once a hyporesponsive state has been established toward the peptide antigen, or at least a shift toward desensitisation has occurred, the method entails administration of a secondary composition comprising substantially a whole antigen from which the peptide antigen was obtained or derived from. Administration of the secondary composition is carried out in such a way as to take advantage of the tolergeneic environment established by use of the primary composition, where it is now possible to establish tolerance to the whole antigen. The secondary composition can be coadministered with another, different antigen (i.e., different to both the first polypeptide antigen and the whole antigen from which it was obtained or derived), wherein it is desired to also desensitise the individual to the second antigen.

Here again, the antigen employed in step (1), that is, in the primary composition, may be any suitable antigen. Typically, it is an allergen. It is not necessary for the individual to be allergic to the antigen specified in step (1) but it may be preferred. In this regard, the individual need not be atopic and need not have generated an IgE response to the whole antigen. However, it is necessary for the individual to have been previously exposed to at least the first antigen. Typically, the individual has mounted a T cell response (Th1 or Th2) to the first antigen from which the peptides are derived, and have specific T cells (of any kind) present that may then be rendered “tolerogenic” by peptide treatment. Thus, in one embodiment, it is determined whether the individual has previously been exposed to an antigen before commencing treatment. This can be determined by measuring serum antibodies to the antigen and/or by carrying out a T cell proliferation assay and/or cytokine assay. It will be appreciated that it is preferred that the peptides administered in the first step are ones derived from a ubiquitous allergen such as house dust mite allergen or cat dander allergen or tree or grass pollen allergen in which case it may not be necessary to test the individual.

By “substantially whole antigen” or “substantially whole allergen” we include the meaning that at least 50% of the antigen or allergen molecule is present, preferably at least 70% or 80% or 90% and, most preferably, all of the antigen or allergen. The percentage of the antigen or allergen can be determined by reference to the number of amino acids in the substantially whole antigen or allergen and the number in the native antigen or allergen.

Typically, the substantially whole antigen or allergen has at least 50% of the T cell epitopes of the whole antigen or allergen, preferably at least 70% or 80% or 90% and most preferably all of the T cell epitopes of the native antigen or allergen.

In both the first and second aspects (methods) of the invention, the T-cell-epitope-containing peptide, or composition of T-cell-epitope-containing peptides, may be administered in step (1) as a single dose. However, it is preferred that the peptide or plurality of peptides is administered as a course so that an optimal state of hyporesponsiveness is achieved before administering the composition or substantially whole allergen in step (2).

In preferred embodiments, administration of the primary composition is carried out in an escalating dose regime, that is, escalating doses of the primary composition comprising the peptide or peptides can administered over a suitable period of time. Suitable escalating doses of the peptide or peptides may be determined by the person skilled in the art, for example, by carrying out symptom-free updosing which results in tolerance (i.e., reduced reactivity to whole injected antigen in the skin). The symptom tested may conveniently be an isolated late asthmatic reaction. Skin tests to whole antigen are generally a good way of assessing induction of tolerance, with or without in vitro T cell proliferation and/or cytokine assays.

For escalating dosage regimes, typically 5 μg of each peptide is initially administered, and then increasing doses of the primary composition are given until a cumulative dose of 90 μg has been administered. The increasing doses of peptide are typically administered with intervals of several days (e.g., 10 to 60 days, but typically 10 to 20 days) between each administration. The total cumulative dose may thus be given over a period of 8 to 16 weeks.

Accordingly, in a preferred embodiment, the suitable course of administration may be doses of 5 μg, 10 μg, 25 μg and then 50 μg of each peptide with intervals of between 3 days to 8 weeks, preferably between 10 days and 4 weeks, and more preferably around 10 to 14 days, between the doses given. The doses may start lower, such as 0.1 μg, or 1.0 μg. Conveniently, a “maintenance” dose may be administered subsequently, such as one or two or three doses of 100 μg with a dose interval of two weeks.

Typically, the lowest initial dose will be between about 0.01 to 0.1 μg, more particularly between about 0.05 to 0.1 μg. The highest initial dose may be higher for non-allergic individuals (for example in treating autoimmunity where there is no risk of anaphylaxis) and may be in the mg range, but preferably will be 100 to 500 μg.

The course of administration that gives rise to the state of hyporesponsiveness may be any suitable course. For example, in commonly owned International Publication WO 99/34826 (PCT/GB99/00080), a method is disclosed for defining the dose for therapy which involves injecting peptides into groups (cohorts) of asthmatic subjects until 50% of them develop a late asthmatic reaction (defined as a greater then 20% fall in forced expiratory volume in one second (FEV1)). From this “upper dose” it is possible to work backwards to establish the starting dose. Typically, if the 50% LAR dose is 5 μg of peptide, the initial therapy dose should be one 50^{th} of that i.e., 0.1 μg.

By “state of hyporesponsiveness to the antigen in the individual” we mean that the individual becomes hyporesponsive to the antigen, for example, as measured by a reduction in size of skin reactions to the antigen. Hyporesponsiveness can readily be determined using well-known clinical parameters. The hyporesponsive individual will contain antigen-specific T cells since their ability to proliferate in response to antigen is reduced as is their ability to secrete disease-associated cytokines such as, in the case of allergic reactions, IL-4 and IL-5 and, possibly IFN-γ. It will be appreciated that the state of hyporesponsiveness includes antigen-specific non-responsiveness, i.e., tolerance.

Challenge testing; such as intradermal injection or inhaled challenge (in an asthmatic) or intraocular administration (for allergic conjunctivitis) may be used. Intradermal testing may be simplest and works on individuals with IgE-mediated allergy. For individuals who have been exposed, but are not allergic, it is preferred to use in vitro proliferation and/or cytokine assays to show down regulation of T cell responses.

It may be advantageous to administer, at or around the time of the administration of the secondary composition or substantially whole antigen in steps (2) and/or (3), and at a suitable dose (eg 1 ng/kg to 10 μg/kg) immunomodulating compositions, such as interleukin-10 (IL-10) or transforming growth factor-β (TGF-β) or interferon-α (IFN-α) or combination thereof may be desirable to aid the production of a hyporesponsive or tolerogenic state. A relatively high dose of IL-10 or TGF-β or IFN-α may be administered.

In relation to any of the previously disclosed methods of the invention, the polypeptide antigen to which the individual is desensitised is any one of an allergen, an autoantigen or a transplant antigen. The allergen may be any allergen disclosed herein above. In addition, the autoantigen may be any autoantigen disclosed herein above.

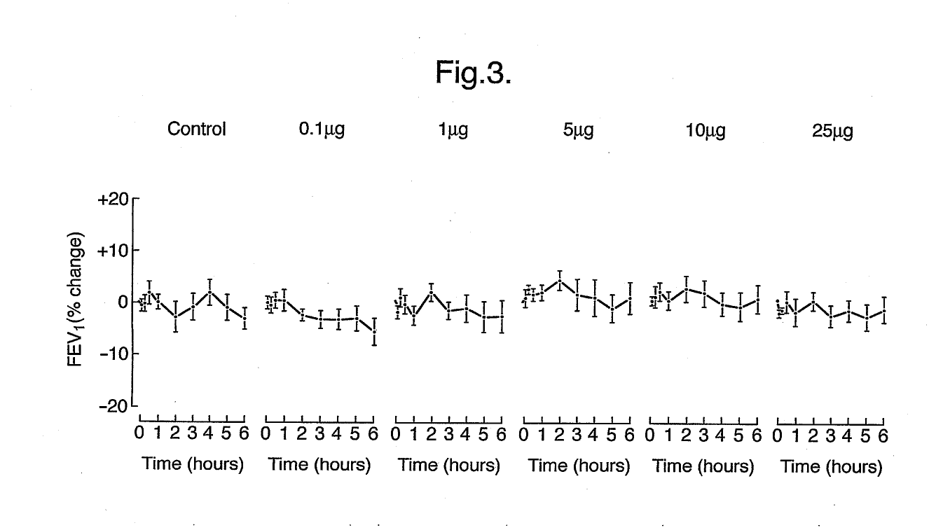

Thus, in a preferred embodiment of the invention, the individual is administered a course of peptides obtained or derived from the allergen to which he is to be desensitised, which leads to a state of hyporesponsiveness or tolerance in the individual to the allergen. There is a window within the state of hyporesponsiveness or tolerance which, following the first administration, takes, typically around two weeks to develop, for example between 10 and 14 days, and becomes optimal after which it is gradually lost unless further peptide or whole protein doses are administered. Further doses may improve the degree of tolerance induced as manifest by a more marked reduction in allergen reactivity following challenge test, after a course of several peptide administrations versus just one (see, for example, FIG. 3). The state of hyporesponsiveness may last for different lengths of time following the administration, depending on the number of administrations of peptides, and during this time the individual is administered whole allergen corresponding to the peptides used in producing the state of hyporesponsiveness. The optimal window may be between 2 weeks and 2 months. Typically, a single administration (injection) of peptide lasts for several months in giving only 50% reactivity after four months (ie the subsequent administration (eg injection) of peptide(s) may give a late phase response (eg late asthmatic response) of about 50% of the original LPR, when the second administration (eg injection) is four months after the first). Typically, full reactivity (responsiveness) has returned after 8 to 12 months. However, with multiple injections and a higher overall dose of peptide, hyporesponsiveness may last longer.

Typically, a series of administrations of compositions comprising increasing doses (with or without a “maintenance” dose) of peptides are carried out over a prescribed period of time with an interval between administrations of several days (we have now shown that the optimal interval is approximately 2-8 weeks). Following peptide administration, administration of the whole protein (with or without other proteins to which the induction of hyporesponsiveness is also desirable) is carried out. This may be followed by further injections of the whole protein antigen. Finally, a challenge test may be performed in which the subject's response to challenge with the whole protein antigen is measured.

Thus, as an example and as described in more detail in the Experimental Section herein below, an individual is selected who is known to be allergic to cat dander and, in particular, the cat dander allergen, Fel d 1. The individual is administered a course of T-cell-epitope-containing peptides of the Fel d 1 allergen (for example, a composition comprising the following peptides:

| | |
|---|---|
| EICPAVKRDVDLFLTGT; | (SEQ ID NO. 1) |
| LFLTGTPDEYVEQVAQY; | (SEQ ID NO. 2) |
| EQVAQYKALPVVLENA; | (SEQ ID NO. 3) |
| KALPVVLENARILKNCV; | (SEQ ID NO. 4) |
| RILKNCVDAKMTEEDKE; | (SEQ ID NO. 5) |
| KMTEEDKENALSLLDK; | (SEQ ID NO. 6) |
| KENALSLLDKIYTSPL; | (SEQ ID NO. 7) |
| LTKVNATEPERTAMKK; | (SEQ ID NO. 8) |
| TAMKKIQDCYVENGLI; | (SEQ ID NO. 9) |
| SRVLDGLVMTTISSSK; | (SEQ ID NO. 10) |
| ISSSKDCMGEAVQNTV; | (SEQ ID NO. 11) |
| and | |
| AVQNTVEDLKLNTLGR. | (SEQ ID NO. 12)) |

A state of hyporesponsiveness to Fel d 1 is generated in the individual, as can be tested by, for example, skin testing the individual by intradermal injection of Fel d 1. One week after the administration of the last dose of the peptide, and while still in a state of hyporesponsiveness, the individual is administered whole Fel d 1 allergen. Typically, a small quantity of Fel d 1 is administered, for example 4 or 5 ng. Typically, this is followed by higher doses, for example 100 ng to 100 μg, and possibly more, which are typically delivered in 2 weekly intervals to allow the previously administered Fel d 1 to expand the tolerogenic T cell pool before administration of the further dose.

It will be appreciated that similar treatment regimes can be used for other antigens. If the antigen is an autoantigen it may be preferable to use higher doses, e.g., 1 to 10 mg of autoantigen.

It will be appreciated that in the methods of the invention, the whole antigen that is administered to the individual is part of the therapeutic regime and not to test sensitivity to the whole antigen. Thus, the method of desensitising the individual specifically includes the step of administering the whole antigen for the purpose of increasing the desensitivity to the antigen and not for the purpose of testing the effect of the administration of peptides alone on the sensitivity of the individual to whole antigen.

It is preferred if the method of the invention is not one which includes administration of a primary composition comprising an allergen that an individual may be seriously allergic to, for example, a primary composition comprising peptides of bee venom phospholipase A2 followed by administration of whole bee venom phospholipase A2, or a primary composition comprising peptides of peanut allergen followed by administration of whole peanut allergen.

In a more preferred embodiment of the invention, the individual is administered a course of peptide antigens to which he or she previously has been exposed and which lead to a state of hyporesponsiveness or tolerance in the individual to the antigen and then the individual is administered, while still in the state of hyporesponsiveness, the whole antigen corresponding to, that is, from which the peptide or peptides used in producing the state of hyporesponsiveness were obtained or derived from and a further, different whole antigen, e.g., an allergen. Typically, between 1 ng and 10 mg of whole antigen is administered, for example, between 10 ng and 1 mg, or between 100 ng and 100 μg, for example between 1 and 10 μg. In this way, the individual can be desensitised to the further, different whole antigen (and also to the whole antigen corresponding to the peptide even if the individual is not allergic to that whole antigen), in the sense that the individual has T cells that can recognise the whole antigen corresponding to the peptide, i.e., has “antigen-primed” T cells regardless of their Th1/Th2 phenotype.

Typically, the individual is allergic to the further, different whole antigen and it is to this antigen that the individual is to be desensitised. It will be appreciated that the method can be used to desensitise the individual to one or two or three or four or more antigens using this technique. Thus, as an example, treatment for an individual allergic to cat, house dust mite and grasses can entail administration of a primary composition comprising peptides of cat dander allergen Fel d 1, administered in a course suitable to induce a state of hyporesponsiveness to the cat dander antigen. While still in the state of hyporesponsiveness the individual is administered a secondary composition comprising whole cat allergen (Fel d 1), whole house dust mite allergen and whole grass allergen, or a plurality of secondary compositions comprising each whole allergen. By using this method the individual can be desensitised to cat allergen, house dust mite allergen and grass allergen.

It will be appreciated that it is not essential for all of the whole antigens to be administered simultaneously, although this is particularly preferred for convenience and to make best use of the tolerogenic environment. If the two different antigens are not administered simultaneously or substantially simultaneously, it is much preferred if the antigen corresponding to the peptides is administered first and the second antigen is administered at the same site as the first antigen.

It will be appreciated that it may be desirable to administer additional doses of one or more of the whole antigens. Typically, the additional doses would be administered twice weekly or monthly. Additional doses can be given if, following testing of the individual for reactivity to the antigens by e.g., antigen challenge (by measuring size of reaction) it is suggested that further doses are required.

The methods of the invention thus provide the opportunity to treat individuals who are allergic to multiple allergens in a relatively short period of time. The method is also beneficial in the treatment of individuals who are very seriously allergic to some things but more mildly allergic to others. Thus, for example, the peptide or polypeptide antigen administered in the primary composition (in step (1)) can be directed at a mild allergen and then the selected, more severe polypeptide antigen could be administered using escalating doses of that severe allergen. Thus, as an example, where the individual to be treated is mildly allergic to cat dander but is very seriously allergic to peanuts, the individual can be treated with a course of T-cell-epitope-containing peptides of cat dander allergen to induce a state of hyporesponsiveness. The individual would then have a dose of whole cat allergen at the same time as a very low dose of whole peanut allergen. The individual is subsequently administered escalating doses of whole peanut allergen together with further doses of cat allergen.

It will be appreciated that combinations of peptides and whole allergens may be selected in order to tailor the treatment of the individual depending on the needs of the individual (e.g., to which allergens the individual needs desensitising and to which allergens the individual is mildly allergic or severely allergic).

In addition, the methods of the invention, particularly the first method, can be used to desensitise the individual to an autoantigen, that is, where the selected polypeptide antigen is an autoantigen. The autoantigen may be any associated with any autoimmune disease. The following is a non-exhaustive list of autoimmune diseases and implicated autoantigens that may be used in the method of the invention: Multiple sclerosis—MBP (myelin basic protein), PLP (proteolipid protein), and/or MOG (myelin oligodendrocyte glycoprotein); Diabetes—GAD (glutamic acid decarboxylase), insulin, and/or IA-2 (a protein tyrosine phosphatase-like molecule); Rheumatoid Arthritis—collagen and/or HSP's (heat shock proteins); Thyroiditis—thyroglobulin; Systemic Lupus Erythromatosus—histone proteins and/or immunoglobulin heavy chain; Behcet's disease—Sag (S antigen from the eye), HLA-B44, HLA-B51, and/or HSP65; Coeliac Disease/Dermatitis herpetiformis—gliadin (rather than use whole gliadin, it may be useful to use a fraction of gliadin which is able to down regulate gliadin-specific T-cell proliferation. A suitable fraction may be the α fraction disclosed in Maurano et al (2001) Scand. J. Immunol. 53:290-295); and Myasthenia gravis-acetyl choline receptor.

The methods of the invention can also be generally applied to treat Th1 type autoimmunity, such as rheumatoid arthritis, diabetes and multiple sclerosis (MS) in individuals that are not allergic. Thus, in one embodiment an individual has MS but is not allergic to cats. However, the individual will have been exposed to cat dander allergen. The individual is given a suitable course of cat dander peptides to induce a suitable hyporesponsive or tolerogenic environment to the cat dander allergen and then a secondary composition comprising cat dander peptides or substantially whole cat dander allergen is coadministered with myelin basic protein. In this way, the cat-specific T cells, present in the environment to which the myelin basic protein is injected, will down-regulate the individual's inappropriate immune response to myelin basic protein-specific cells, thereby treating the MS.

The methods of the invention can also be generally applied to treat graft or transplant rejection. For example, the transplant antigen (selected polypeptide antigen to which the individual would be desensitised to) may be a HLA-A2 molecule or other molecule encoded by an MHC gene. Approximately 50% of Caucasians express the Class I MHC molecule HLA-A2. This is a very immunogenic molecule and when a non-A2 recipient gets an A2 graft there is a strong T and B cell response to the molecule. Host T cell receptors recognize a peptide from HLA-A2 presented by their own MHC molecules and react (this is similar to Behcet's disease mentioned above where a peptide common to both the HLA-B44 and HLA-B51 molecules appears to act as an antigenic peptide). Thus, therapy with the peptide may lead to a state of successful engraftment.

Suitably, the methods of the invention may be used to desensitise to any protein that causes a pathogenic T cell response, including proteins from latex or from the mite, Trichopyton tonsurans, which cause Th1 hypersensitivity in the skin.

It will be appreciated that the methods of the invention are particularly suited for desensitising an individual to more than one polypeptide antigen simultaneously.

The route of administration used to administer any one of the primary composition, secondary composition and/or a composition comprising the first polypeptide antigen or a larger molecule containing the first polypeptide antigen can be carried out using any known technique including intradermal injection, subcutaneous injection, intramuscular injection, intravenous injection, transdermal, intranasal, oral including inhaled via the mouth, intraocular or intrathecal administration techniques.

Preferred routes of administration are intradermal, intramuscular, intravenous and subcutaneous injections, wherein intradermal or subcutaneous injection routes are particularly preferred. Another preferred route of administration is by using a transdermal particle injection technique.

It is particularly preferred that the primary compositions, secondary compositions, and any other compositions (e.g., those coadministered with the secondary compositions) are administered to substantially the same site in order to make best use of the localised, tolerogenic environment generated by administration of the polypeptide antigen from the primary composition or larger molecule containing the polypeptide antigen from the primary composition. However, the skilled artisan will understand, upon reading the instant specification, that the primary and secondary compositions can be administered to different sites, and using different administration techniques.

It will be appreciated by the skilled artisan, upon reading the instant specification, that it may be desirable to administer the secondary composition over a period of time and in a course of administration, for example, using a regime of escalating doses. In this way it is believed that the hyporesponsiveness or tolerogenic environment may be expanded. Typically, the escalating doses will fall within the range of about 1 ng to 1 mg, typically 10 ng to 100 μg, or more typically 100 ng to 10 μg.

If multiple administrations are conducted in the second step of the invention, it is preferred that the administrations are to substantially the same site in the individual. Typically, when a plurality of selected polypeptide antigens are administered in the secondary composition (or secondary compositions), the antigens are administered simultaneously, to the same site, and typically as a single combined composition. However, if multiple antigens are selected for desensitisation, it is of course possible to administer a plurality of different secondary compositions to two or more different sites.

Whilst it may be possible for the peptides, polypeptides, or substantially whole antigens to be presented in raw form, it is preferable to present them as a pharmaceutical formulation for use in the various compositions administered to the individual in the practice of the instant methods. A pharmaceutical formulation comprises one or more peptide, polypeptide, or substantially whole antigen as defined herein together with one or more auxiliary substances such as pharmaceutically acceptable carriers or vehicles therefor and optionally one or more other ingredients such as an excipient or stabilizer. The various carriers, vehicles, and excipients must be “acceptable” in the sense of being compatible with the other ingredients of the formulation and not deleterious to the recipient thereof. Typically, carriers or vehicles for injection, and the final formulation, are sterile and pyrogen-free.

More particularly, auxiliary substances, such as wetting or emulsifying agents, pH buffering substances and the like, may be present in the compositions of the present invention as an excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer for the peptide, polypeptide, protein or other like molecules in the composition. Examples of suitable carriers that also act as stabilizers for peptides, polypeptides and proteins include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991).

Formulations useful in the practice of the instant methods include those suitable for oral (e.g., sublingual or inhaled), parenteral (including subcutaneous, transdermal, intradermal, intramuscular and intravenous and rectal), or transdermal administration, although the most suitable route may depend upon for example the condition and disorder of the recipient. The formulations may conveniently be presented in unit dosage form and may be prepared by any of the methods well known in the art of pharmacy. The methods disclosed herein will thus normally include the step of bringing into association a composition of the present invention as herein defined or a pharmacologically acceptable salt or solvate thereof (“active ingredient”) with a carrier, vehicle or excipient which constitutes one or more accessory ingredients.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Formulations for inhalation may be presented in any of the ways known to be effective e.g., metered dose inhalers.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

The peptides, polypeptides or substantially whole antigens/allergens of the invention may also be administered intranasally, by inhalation, orally or via injection at a dose of from 0.0001 to 1 μg/kg per dose. Preferred are doses in the region of 10 ng to 10 mg per human individual, such as 10 μg to 1 mg or 100 μg to 1 mg, advantageously about 80 μg.

Preferred unit dosage formulations are those containing an effective dose of the active ingredient as herein described, or an appropriate fraction thereof. It will be appreciated that when a course of administration of different doses of the peptide or polypeptides in step (1), that is, in the primary composition, or the composition or compositions used in step (2), that is, in the secondary composition or other compositions are to be administered, unit dosage formulations for each dose in the course may be prepared. Thus, typically, for a course of peptides or polypeptides to be administered, a unit dosage of the peptide is available for each, typically escalating, dose.

In some preferred embodiments, one or more of the primary composition, secondary composition and any further composition (e.g., the composition comprising the first polypeptide antigen or a larger molecule containing the first polypeptide antigen) can be formulated as a particulate (powdered) composition. More particularly, formulation of particles comprising the peptide, polypeptide or whole antigen can be carried out using standard pharmaceutical formulation chemistries. For example, the antigen molecules can be combined with one or more pharmaceutically acceptable excipient or vehicle to provide a suitable formulated composition.

The formulated compositions can then be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in International Publication No. WO 97/48485.

These methods can be used to obtain nucleic acid particles having a size ranging from about 0.01 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Similarly, particles of selected adjuvants having a size ranging from about 0.1 to about 250 μm, preferably about 0.1 to about 150 μm, and most preferably about 20 to about 60 μm; a particle density ranging from about 0.1 to about 25 g/cm³, and a bulk density of preferably about 0.5 to about 3.0 g/cm³, and most preferably about 0.8 to about 1.5 g/cm³ can be obtained.

Single unit dosages or multidose containers, in which the particles may be packaged prior to use, can comprise a hermetically sealed container enclosing a suitable amount of the particles comprising the antigen of interest. The particulate compositions can be packaged as a sterile formulation, and the hermetically sealed container can be designed to preserve sterility of the composition until use in the methods of the invention. If desired, the containers can be adapted for direct use in a needleless syringe system. Such containers can take the form of capsules, foil pouches, sachets, cassettes, and the like. Appropriate needleless syringes are described herein and are otherwise known in the art.

The container in which the particles are packaged can further be labelled to identify the composition and provide relevant dosage information. In addition, the container can be labelled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

Following their formation, the particulate composition (e.g., powder) can be delivered transdermally to the individual's tissue using a suitable transdermal particle injection technique. Various particle acceleration devices suitable for transdermal particle injection are known in the art, and will find use in the practice of the invention. A particularly preferred transdermal particle injection system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect needleless syringe device”). Other needleless syringe configurations are known in the art and are described herein. Preferably, the particulate compositions will be delivered using a needleless syringe system such as those described in International Publication Nos. WO 94/24263, WO 96/04947, WO 96/12513, and WO 96/20022. Delivery of particles from such needleless syringe systems is typically practised with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these needleless syringe systems can be provided in a preloaded condition containing a suitable dosage of the particles comprising the peptide, polypeptide or whole antigen of interest. The loaded syringe can be packaged in a hermetically sealed container, which may further be labelled as described above.

The powdered compositions are administered to the individual to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid molecule per dose, depends on the subject to be treated. Doses for physiologically active peptides and proteins generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

In a third aspect of the invention, we provide a therapeutic system (or, as it may be termed, a kit of parts) for desensitising an individual to one or more polypeptide antigens. The system comprises (1) a T-cell-containing peptide, or a plurality of T-cell-epitope-containing peptides, of an antigen and (2) a composition which contains the T cell epitope of a peptide as defined in (1) and further contains a T cell epitope of the one or more polypeptide antigens to which the individual is to be desensitised wherein the composition is capable of remaining substantially at the site of its administration.

Put in another way, in a third aspect of the invention, we provide a therapeutic system for desensitising an individual to one or more polypeptide antigens. The system comprises a primary composition comprising a first polypeptide antigen containing a T cell epitope, and a secondary composition comprising the first polypeptide antigen and at least one second, different antigen to which the individual is to be desensitised.

Typically, compositions capable of remaining at the site of administration are, or are formulated to be able to localise at the site of administration. Suitable compositions and formulations are described above in relation to the methods of the invention. Conveniently, the secondary composition comprises a substantially whole antigen corresponding to the peptide or peptides present in the primary composition. Preferably the polypeptide antigen is an allergen. Typically, the secondary composition comprises a T cell epitope of the first polypeptide antigen and at least one T cell epitope of a different polypeptide antigen. The secondary composition may comprise a fusion of a T-cell-epitope-containing peptide and a different substantially whole polypeptide antigen.

In a fourth aspect of the invention, we provide a therapeutic system (or, as it may be termed, a kit of parts) for desensitising an individual to one or more polypeptide antigens each of which contains a T cell epitope. The system comprises (1) one or more peptide antigens containing T cell epitope, (2) a composition which contains a T cell epitope of a peptide defined in (1), and (3) a composition which contains a T cell epitope of a different polypeptide antigen wherein the compositions defined in (2) and (3) are capable of localising at the site of their administration. Typically, the composition defined in (2) is the substantially whole antigen corresponding to the peptide or peptides as defined in (1) and the composition defined in (3) is a substantially whole antigen. Preferably, the antigen is an allergen.

In a fifth aspect of the invention, we provide a therapeutic system (or, as it may be termed, a kit of parts) for desensitising an individual to one or more polypeptide antigens, the system comprising one or more peptide antigen containing a T cell epitope and a first substantially whole antigen corresponding to the peptide antigen or antigens. Typically, the system further comprises a second substantially whole polypeptide antigen that is different to the first substantially whole allergen. Preferably, the antigen is an allergen.

Thus, the invention includes various therapeutic systems (or kits of parts) for desensitising an individual to a selected antigen (e.g., an allergen). The systems contain a peptide antigen as defined herein above and substantially whole antigen, wherein both components are to be used in a treatment regime for desensitising the individual to the antigen. Typically, the kit of parts includes instructions for using the two components in a therapeutic regime. Suitably, the peptide antigen is one which, when administered to a suitable individual gives rise to a state of hyporesponsiveness in the individual to the antigen from which it is derived. An example of this type of therapeutic system is one containing a Fel d 1 peptide such as one or more of the following peptides: EICPAVKRDVDLFLTGT (SEQ ID NO. 1); LFLTGTPDEYVEQVAQY (SEQ ID NO. 2); EQVAQYKALPVVLENA (SEQ ID NO. 3); KALPVVLENARILKNCV (SEQ ID NO. 4); RILKNCVDAKMTEEDKE (SEQ ID NO. 5); KMTEEDKENALSLLDK (SEQ ID NO. 6); KENALSLLDKIYTSPL (SEQ ID NO. 7); LTKVNATEPERTAMKK (SEQ ID NO. 8); TAMKKIQDCYVENGLI (SEQ ID NO. 9); SRVLDGLVMTTISSSK (SEQ ID NO. 10); ISSSKDCMGEAVQNTV (SEQ ID NO. 11); AVQNTVEDLKLNTLGR (SEQ ID NO. 12); and peptides substantially homologous to any one or more of SEQ ID NOs 1-12, and whole Fel d 1. Thus, the Fel d 1 peptide and the whole Fel d 1 are to be used therapeutically in a treatment regime. This type of system may optionally contain whole allergen (e.g., whole Fel d 1) to establish the effectiveness of the desensitisation using the other components.

The invention also includes a therapeutic system (or kit of parts) which contains a course of peptides of an antigen (e.g., an allergen) as defined and substantially whole antigen. The system may include a course of peptides and a course of substantially whole antigen. The course of peptides of an antigen is one which, when administered to a suitable individual, gives rise to a state of hyporesponsiveness in the individual to the antigen from which it is obtained or derived from. Typically, as described above, the course of peptides may be a course of peptides of escalating dose. In one embodiment, the therapeutic system may contain a single peptide antigen containing a T cell epitope but present separately packaged in a series of escalating concentrations or in escalating unit dosages for administration in escalating doses to the individual. In a further embodiment, the system may contain different peptide antigens either separately packaged or packaged together (e.g., present in the same sterile and pyrogen free aqueous solution). Suitably, in some embodiments, the plurality of peptides are packaged in a series of escalating concentrations or escalating unit dosages for administration in escalating doses to the individual. Other suitable combinations may be readily devised by the person skilled in the art given the teachings herein.

The allergen may also be packaged in a series of escalating concentrations or in escalating unit dosages for administration in escalating doses to the individual.

Thus, as an example, a therapeutic system for desensitising an individual to cat dander contains any one or more peptides selected from EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOs 1-12. separately packaged in unit dosages of 5 μg, 10 μg, 25 μg and 50 μg and whole Fel d 1 antigen separately packaged in unit dosages of 1 ng to 50 μg. Suitable instructions may be provided to indicate to the physician how to administer the peptides in order to give an optimal state of hyporesponsiveness to Fel d 1 before administration of the whole Fel d 1 allergen. Similar therapeutic systems and kits may be devised for desensitisation to other antigens.

In a preferred embodiment, the invention includes a therapeutic system for desensitising an individual to multiple antigens (e.g., multiple allergens). The system contains a peptide antigen to which the individual has been exposed previously, substantially whole antigen corresponding to the peptide, that is, from which the peptide antigen was obtained or derived, and one or more substantially whole antigens to which the individual is to be desensitised. Typically, the therapeutic system contains the peptide, peptides, or course of peptides as described above and the antigen corresponding to the peptide, or peptides. However, in addition, the therapeutic system contains the one or more whole antigens to which the individual is to be desensitised.

Thus, in one embodiment the therapeutic system is for desensitising the individual to peanut allergen. The individual has been exposed to cat dander allergen (but may not be allergic to cat dander) but is allergic to peanut allergen. The system contains a course of Fel d 1 peptides for producing a state of hyporesponsiveness, whole Fel d 1, and course of whole peanut allergen of escalating dosages.

In another embodiment the therapeutic system is for desensitising the individual to cat dander, house dust mites and grass pollen. The individual is allergic to cat dander allergen, house dust mite allergen and grass pollen. The system contains a course of Fel d 1 peptides for producing a state of hyporesponsiveness, whole Fel d 1 and whole house dust mite allergen and whole grass pollen allergen.

In a sixth aspect of the invention, we provide for the use of a T-cell epitope-containing peptide of an antigen (e.g., an allergen) to which the individual has been exposed, in the manufacture of a medicament for generating in the individual a state of hyporesponsiveness to the antigen to allow desensitisation to one or more polypeptide antigens each of which contains a T cell epitope. Preferably, the antigen is an allergen.

In a seventh aspect of the invention, we provide for the use of a composition which contains a T cell epitope of one or more polypeptide antigens to which the individual is to be desensitised, in the manufacture of a medicament for administration to an individual who has been administered a T-cell-epitope-containing peptide, or a course of T-cell-epitope-containing peptides, of an antigen, such as an allergen, to which the individual has been exposed, in order to generate in the individual a state of hyporesponsiveness to the antigen and, if the composition does not contain a T cell epitope of the peptide or peptides administered, the individual has further been administered a composition which contains said T cell epitope, wherein the compositions are substantially localised at the site of administration.

In an eighth aspect of the invention, we provide for the use of a T-cell-epitope-containing peptide of an antigen, preferably an allergen, to which the individual has been exposed, in the manufacture of a medicament for generating in the individual a state of hyporesponsiveness to the antigen to allow for desensitisation to one or more substantially whole allergens.

In a ninth aspect of the invention, we provide for the use of a substantially whole first antigen, preferably an allergen, to which an individual has been exposed, in the manufacture of a medicament for desensitising the individual to one or more polypeptide antigens wherein the individual has been administered a T-cell-epitope-containing peptide, or a course of T-cell-epitope-containing peptides, of the first antigen and subsequently the individual is administered substantially whole antigen to which the individual is to be desensitised.

In a tenth aspect of the invention, we provide for the use of a substantially whole antigen, preferably an allergen, to which an individual has been exposed in the manufacture of a medicament for desensitising the individual to the antigen wherein the individual has been administered a T-cell-epitope-containing peptide, or a course of T-cell-epitope-containing peptides, of the antigen.

### C. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

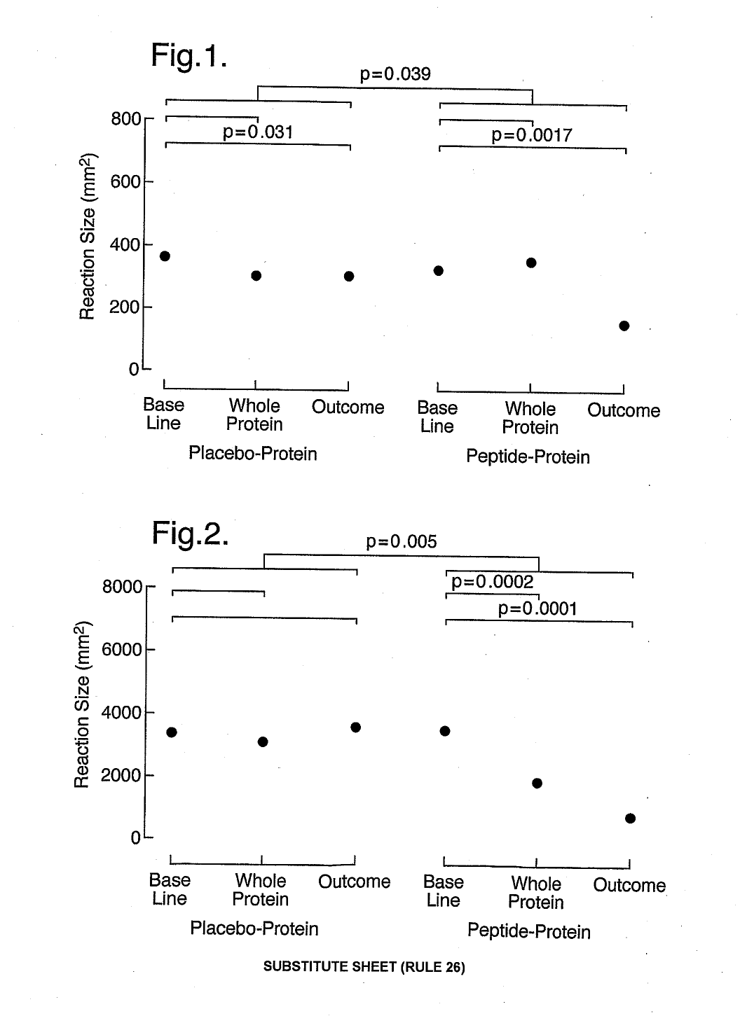

FIG. 1. Administration of peptides followed by whole protein reduces the cutaneous early phase reaction to Fel d 1. In a placebo-controlled, double blind clinical trial, subjects were injected intradermally with whole Fel d 1 protein and the size of the reaction at 15 minutes measured (baseline). A series of injections were administered of peptides derived from the sequence of Fel d 1. The dose of peptides started at 5 μg of each peptide and increased until a cumulative dose of 90 μg had been administered. Approximately 2-4 weeks later the whole protein was injected (whole protein). 3-6 months later whole protein challenge was performed and the magnitude of the skin reaction at 15 minutes defined (outcome).

FIG. 2. Administration of peptides followed by whole protein reduces the cutaneous late-phase reaction to Fel d 1. In a placebo-controlled, double blind clinical trial, subjects were injected intradermally with whole Fel d 1 protein and the size of the reaction at 6 hours measured (baseline). A series of injections were administered of peptides derived from the sequence of Fel d 1. The dose of peptides started at 5 μg of each peptide and increased until a cumulative dose of 90 μg had been administered. Approximately 2-4 weeks later the whole protein was injected (whole protein). 3-6 months later whole protein challenge was performed and the magnitude of the skin reaction at 6 hours defined (outcome).

FIG. 3. Peptides from the cat allergen Fel d 1 were administered intradermally at 2 weekly intervals in the following dose schedule: 0.1 μg, 1.0 μg, 5.0 μg, 10.0 μg, 25.0 μg. No isolated late asthmatic reactions were observed (n=8 subjects; pooled data). EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12).

### Example 1

### Desensitisation to Fel d 1

In this example, in a double-blind, placebo-controlled study, 16 subjects were injected intradermally with a mixture of the peptides: EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12) (starting at 5 μg of each peptide and increasing in dose until a cumulative dose of 90 μg was achieved) derived from the sequence of the major cat allergen Fel d 1. A control group of 8 individuals received diluent alone (placebo). Subsequently (after approximately one month) they were injected intradermally with the whole protein. Finally after 3-6 months their response to intradermal challenge with the whole protein was assessed in terms of the size (in mm²) of the skin reaction measured at 15 minutes and 6 hours post injection. The reactions to the injected proteins were compared to the reaction to equivalent protein challenges performed before the peptide injection.

FIG. 1 shows that injection of peptides followed by protein leads to a statistically significant reduction in the size of the skin response to challenge with the whole protein at 15 minutes.

FIG. 2 shows that injection of peptide alone is able to cause a significant reduction in the size of the reaction to the whole protein injection, but that the effect of the peptide injections and the protein injection is even greater than the peptide injection alone, since the combination of the two markedly reduce the size of the reaction to the 3-6 month “outcome” injection measured 6 hours after protein challenge.

### Example 2

### Desensitisation to House Dust Mite Allergen, Der p 1

In this example, subjects are injected intradermally with a mixture of peptides: EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOs 1-12. (starting at 5 μg of each peptide and increasing in dose until a cumulative dose of 90 μg is achieved) derived from the sequence of the major cat allergen Fel d 1. A control group receives diluent alone (placebo). Subsequently (after approximately one month) they are injected intradermally with the whole Fel d 1 protein which is mixed with the house dust mite protein allergen Der p 1. Finally after 3-6 months their response to intradermal challenge with the whole Fel d 1 protein and the whole Der p 1 protein is assessed in terms of the size (in mm²) of the skin reaction measured at 15 minutes and 6 hours post injection. The reactions to the injected proteins are compared to the reaction to equivalent protein challenges performed before the peptide injection.

Skin reaction to both Fel d 1 and to Der p1 are expected to be reduced at 15 minutes after injection and/or 6 hours after injection.

### Example 3

### Desensitisation to House Dust Mite Allergen, Der p 1

In this example, subjects are injected intradermally with a mixture of peptides: EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOs 1-12. (starting at 5 μg of each peptide and increasing in dose until a cumulative dose of 90 μg is achieved) derived from the sequence of the major cat allergen Fel d 1. A control group receives diluent alone (placebo). Subsequently (after approximately one month) they are injected intradermally with an emulsion containing the Fel d 1 peptides administered previously together with a peptide from the house dust mite protein allergen Der p 1. Finally after 3-6 months their response to intradermal challenge with the whole Fel d 1 protein and the whole Der p 1 protein is assessed in terms of the size (in mm²) of the skin reaction measured at 15 minutes and 6 hours post injection. The reactions to the injected proteins are compared to the reaction to equivalent protein challenges performed before the peptide injection.

Skin reaction to both Fel d 1 and to Der p1 are expected to be reduced at 15 minutes after injection and/or 6 hours after injection.

### Example 4

### Desensitisation to Myelin Basic Protein (MBP)

In this example, subjects are injected intradermally with a mixture of peptides EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10); ISSSKDCMGEAVQNTV (SEQ ID NO. 11), AVQNTVEDLKLNTLGR (SEQ ID NO. 12), and peptides substantially homologous to any one or more of SEQ ID NOs 1-12. (starting at 5 μg of each peptide and increasing in dose until a cumulative dose of 90 μg is achieved) derived from the sequence of the major cat allergen Fel d 1. A control group receives diluent alone (placebo). Subsequently (after approximately one month) they are injected intradermally with the whole Fel d 1 protein which is mixed with the autoantigen myelin basic protein (MBP). Finally after 3-6 months their response to intradermal challenge with the whole Fel d 1 protein is assessed in terms of the size (in mm²) of the skin reaction measured at 15 minutes and 6 hours post injection. The induction of hyporesponsiveness to MBP is measured by assessment of symptom scores for example using the “expanded disability status scale” (Kurtze J F. Rating neurological impairment in multiple sclerosis: an expanded disability status scale. (EDSS). Neurology 33; 1444-52 (1983) and/or the “Scripps neurological rating scale” Sharrack B & Hughes R A. Clinical scales for multiple sclerosis. J. Neurol. Sci. 135; 1-9 (1996) and the use of magnetic resonance imaging (MRI) of the brain. Additionally, in vitro T cell responses to MBP are expected to reduce in terms of proliferative capacity and production of IFNγ. The reaction to the injected Fel d 1 protein is compared to the reaction to equivalent protein challenge performed before the peptide injection:

Skin reaction to Fel d 1 is reduced at 15 minutes after injection and/or 6 hours after injection. Furthermore, the symptoms scores recorded with the instruments listed above are expected to show an improvement associated with a reduction in reactivity to MBP.

### Example 5

### Desensitisation to Both Cat and Dog Allergen

In this example, an individual with cat-induced allergic asthma and also with allergic sensitisation to dog allergen(s) was injected, intradermally with a mixture of peptides from the major cat allergen, Fel d 1. The peptides were specifically EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12). The peptides were injected in incremental divided doses at approximately 2 week intervals. The doses were administered in a 100 microlitre volume and contained the following concentrations of each of the 12 peptides: 1 μg, 5 μg, 10 μg, 25 μg, 50 μg, 100 μg and 100 μg in that order.

At various times during the treatment, the individual was also injected intradermally with whole cat dander extract 30 biological units (BU; purchased from ALK, Horsholm, Denmark) and with dog dander extract (30BU) via the same route.

The magnitude of the early-phase (15 minutes) and late-phase (6 hours) allergic skin response to both allergen extracts was measured before and 4 weeks after the course of peptide injections. The magnitude of the reactions was as follows:

TABLE 1

| | |
|---|---|
| EPR (mm²) | LPR (mm²) |

| Allergen | Before | After | Before | After |
|---|---|---|---|---|
| Cat dander | 75 | 128 | 488 | 0 |
| Dog dander | 61 | 55.3 | 644 | 0 |

In addition to the changes in skin late-phase reactivity to the two allergens, the clinical efficacy of the peptide treatment with respect to symptoms was confirmed by improvement in nasal symptom scores following nasal allergen challenge with cat dander extract (dog allergen not done).

The results demonstrate that treatment with peptides derived from one protein can be used to induce hyporesponsiveness not only to the parent protein but to another protein, provided the whole proteins are co-administered during or shortly after administration of a course of peptides.

1-118. (canceled)

119. A method of desensitizing an individual to a second polypeptide allergen or autoantigen, which method comprises the steps of:
(a) parenterally administering a primary composition comprising a first polypeptide allergen or autoantigen, or a portion thereof which comprises a T cell epitope, to the individual in a manner sufficient to generate a hyporesponsive state against the first polypeptide allergen or autoantigen; and
(b) parenterally administering a secondary composition comprising the second polypeptide allergen or autoantigen, or a portion thereof which comprises a T cell epitope to the individual, wherein said secondary composition is coadministered with the first polypeptide allergen or autoantigen or said portion thereof, whereby the hyporesponsive state generated in step (a) and said coadministration are sufficient to desensitize the individual to the second polypeptide allergen or autoantigen; wherein:
(i) the individual has been previously exposed to the first polypeptide allergen or autoantigen; and
(ii) the first polypeptide allergen or autoantigen is different from the second polypeptide allergen or autoantigen.

120. The method of claim 119 wherein a plurality of secondary compositions are administered to the individual in step (b) and each secondary composition contains a different polypeptide allergen or autoantigen or portion thereof which comprises a T cell epitope, thereby desensitizing the individual to a plurality of polypeptide allergens or autoantigens.

121. The method of claim 119 wherein the individual is allergic to the first polypeptide allergen or autoantigen or the second polypeptide allergen or autoantigen.

122. The method of claim 119 wherein the first or second polypeptide allergen or autoantigen is an aero allergen.

123. The method of claim 122, wherein the aero allergen is selected from the group consisting of a cat dander allergen, a dog dander allergen, a house dust mite allergen, a pollen allergen, a grass allergen, and a food allergen.

124. The method of claim 123, wherein the cat dander allergen is Fel d1.

125. The method of claim 119 wherein the primary composition comprises a plurality of first polypeptide allergens or autoantigens, or portions thereof each containing a T cell epitope.

126. The method of claim 125, wherein the primary composition comprises one or more Fel d 1 peptides selected from the group consisting of EICPAVKRDVDLFLTGT (SEQ ID NO. 1), LFLTGTPDEYVEQVAQY (SEQ ID NO. 2), EQVAQYKALPVVLENA (SEQ ID NO. 3), KALPVVLENARILKNCV (SEQ ID NO. 4), RILKNCVDAKMTEEDKE (SEQ ID NO. 5), KMTEEDKENALSLLDK (SEQ ID NO. 6), KENALSLLDKIYTSPL (SEQ ID NO. 7), LTKVNATEPERTAMKK (SEQ ID NO. 8), TAMKKIQDCYVENGLI (SEQ ID NO. 9), SRVLDGLVMTTISSSK (SEQ ID NO. 10), ISSSKDCMGEAVQNTV (SEQ ID NO. 11), AVQNTVEDLKLNTLGR (SEQ ID NO. 12), and peptides substantially homologous to any one or more of SEQ ID NOS. 1-12.

127. The method of claim 119 wherein step (a) comprises a series of administrations of the primary composition.

128. The method of claim 127, wherein the primary composition is administered to the individual in a series of escalating doses of the first polypeptide allergen or autoantigen or said portion thereof carried out over a period of time.

129. The method of claim 119, wherein the second polypeptide allergen or autoantigen is an allergen selected the group consisting of one or more of the following sources: latex; plants; pollens; fungi; molds; foods; stinging insects; the chirnomidae (non-biting midges); spiders; mites; flies; grain weevil; silkworm; honeybee; non-biting midge larvae; bee moth larvae; mealworm; cockroach; larvae of Tenibrio molitor beetle; and mammals.

130. The method of claim 129, wherein the allergen is selected from the group consisting of Der p 1; Der p 2; Der p 3; Der p 4; Der p 5; Der p 6; Der p 7; Der p 9; Der f 1; Der f 2; Der f 3; Der f 4; Der f 7; Fel d 1 chain 1 or 2; Hey b 1; Hey b 3; Lol p 1; Lol p 2a; Lol p 3; Lol p 5a; Lol p 5b; Lol p isoform 9; Lol p 11; Ole e 1; Par j P2; Par j P5; Par j P8; Par j P9; Par j 1; Phl p 1; Phl p 2; Phl p 5; Phl p 5b; Phl p 5a; VES V 5; VES M 1; VES V 1; VES V 2; VES VI; Bet v 1; Bet v 2; Bet v 3; Bet v 4; Que a I; Car b I; Aln g I; Rubisco; Ara h 1; Amb a 1; Amb a 2; Amb a 1.3; Amb a 1.2; Amb a 1.1; Cry j IB precursor; Cry j IA precursor; Cry j II precursor; Cry j II protein; Cry j I precursor; Can f 1; Can f 2; serum albumin fragment; Equ c1; Equ c 2; Eur m 1; POA P 9; Cr p1; Cr p2; Bla g 2; Bla g 4; and Bla g 5.

131. The method of claim 119, wherein the second polypeptide allergen or autoantigen is an autoantigen associated with any one of the following autoimmune disorders: Multiple Sclerosis; Diabetes; Rheumatoid Arthritis; Thyroiditis; Systemic Lupus Erythematosus; Behcet's Disease; Coeliac Disease; or Myasthenia gravis.

132. The method of claim 131, wherein the autoantigen is selected from the group consisting of: myelin basic protein (MBP); proteolipid protein (PLP); myelin oligodendrocyte glycoprotein (MOG); glutamic acid decarboxylase (GAD); insulin; IA-2 (a protein phosphatase-like molecule); collagen; heat shock proteins (HSP's); thyroglobulin; histone proteins; immunoglobulin heavy chain; S antigen from the eye (Sag); HLA-B44; HLA B51; HSP65; gliadin; and acetyl choline receptor.

133. The method of claim 119 wherein the secondary composition and the first polypeptide allergen or autoantigen or said portion thereof are administered as two separate compositions.

134. The method of claim 133, wherein the secondary composition is administered to a first site and the composition comprising the first polypeptide allergen or autoantigen or said portion thereof is administered to a second site.

135. The method of claim 133, wherein the secondary composition and the composition comprising the first polypeptide allergen or autoantigen or said portion thereof are administered to the same site.

136. The method of claim 119 wherein step (b) comprises administration of a plurality of secondary compositions, each said secondary composition comprising a different polypeptide allergen or autoantigen or portion thereof containing a T cell epitope, whereby the individual is desensitized to more than one polypeptide allergen or autoantigen and wherein the plurality of secondary compositions are combined.

137. The method of claim 136, wherein the plurality of secondary compositions are further combined with the composition comprising the first polypeptide allergen or autoantigen or said portion thereof.

138. The method of claim 136, wherein the plurality of secondary compositions and the composition comprising the first polypeptide allergen or autoantigen or said portion thereof are administered to the same site.

139. The method of claim 136, wherein the secondary compositions are administered to a first site and the composition comprising the first polypeptide allergen or autoantigen or said portion thereof is administered to a second site.

140. The method of claim 119 wherein in step (b) the first polypeptide allergen or autoantigen or said portion thereof is to the individual to an administration site and remains substantially localized at said administration site.

141. The method of claim 140, wherein the first polypeptide allergen or autoantigen or said portion thereof is administered in a slow release formulation or in an oil and water emulsion.

142. The method of claim 119 wherein at least one of said primary composition, secondary composition, or a composition comprising the first polypeptide allergen or autoantigen or said portion thereof is administered using intradermal injection, subcutaneous injection, intramuscular injection, intravenous injection or transdermal administration technique.

143. The method of claim 142, wherein at least one of said primary composition, secondary composition, or the composition comprising the first polypeptide allergen or autoantigen or said portion thereof comprises an aqueous or liquid carrier or vehicle.

144. The method of claim 142, wherein at least one of said primary composition, secondary composition, or the composition comprising the first polypeptide allergen or autoantigen, or portion thereof is provided in dry powdered form.

145. The method of claim 144 wherein said dry powdered composition is administered using a transdermal particle injection technique.

146. The method of claim 119 wherein the primary and secondary composition are administered in the form of separate compositions.

147. The method of claim 129 wherein the second polypeptide allergen or autoantigen is an allergen from one or more of the sources selected from the group consisting of grasses, trees, ragweeds, wasps, houseflies, fruit flies, sheep blow flies, screw worm flies, cats, dogs, horses, cows, pigs, sheep, rabbits, rats, guinea pigs, mice and gerbils.


SEQUENCE LISTING (118 sequences)

SEQ ID NO: 1 - protein, 17 aa, Felis domesticus
EICPAVKRDVDLFLTGT

SEQ ID NO: 2 - protein, 17 aa, Felis domesticus
LFLTGTPDEYVEQVAQY

SEQ ID NO: 3 - protein, 16 aa, Felis domesticus
EQVAQYKALPVVLENA

SEQ ID NO: 4 - protein, 17 aa, Felis domesticus
KALPVVLENARILKNCV

SEQ ID NO: 5 - protein, 17 aa, Felis domesticus
RILKNCVDAKMTEEDKE

SEQ ID NO: 6 - protein, 16 aa, Felis domesticus
KMTEEDKENALSLLDK

SEQ ID NO: 7 - protein, 16 aa, Felis domesticus
KENALSLLDKIYTSPL

SEQ ID NO: 8 - protein, 16 aa, Felis domesticus
LTKVNATEPERTAMKK

SEQ ID NO: 9 - protein, 16 aa, Felis domesticus
TAMKKIQDCYVENGLI

SEQ ID NO: 10 - protein, 16 aa, Felis domesticus
SRVLDGLVMTTISSSK

SEQ ID NO: 11 - protein, 16 aa, Felis domesticus
ISSSKDCMGEAVQNTV

SEQ ID NO: 12 - protein, 16 aa, Felis domesticus
AVQNTVEDLKLNTLGR

SEQ ID NO: 13 - protein, 320 aa, Dermatophagoides pteronyssinus
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 14 - protein, 146 aa, Dermatophagoides pteronyssinus
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 15 - protein, 261 aa, Dermatophagoides pteronyssinus
MIIYNILIVLLLAINTLANPILPASPNATIVGGEKALAGECPYQISLQSSSHFCGGTILDEYWILTAAHCVAGQTASKLSIRYNSLKHSLGGEKISVAKIFAHEKYDSYQIDNDIALIKLKSPMKLNQKNAKAVGLPAKGSDVKVGDQVRVSGWGYLEEGSYSLPSELRRVDIAVVSRKECNELYSKANAEVTDNMICGGDVANGGKDSCQGDSGGPVVDVKNNQVVGIVSWGYGCARKGYPGVYTRVGNFIDWIESKRSQ

SEQ ID NO: 16 - protein, 19 aa, Dermatophagoides pteronyssinus
  MISC_FEATURE (3)..(16): Xaa = any amino acid
KYXNPHFIGXRSVITXLME

SEQ ID NO: 17 - protein, 132 aa, Dermatophagoides pteronyssinus
MKFIIAFFVATLAVMTVSGEDKKHDYQNEFDFLLMERIHEQIKKGELALFYLQEQINHFEEKPTKEMKDKIVAEMDTIIAMIDGVRGVLDRLMQRKDLDIFEQYNLEMAKKSGDILERDLKKEEARVKKIEV

SEQ ID NO: 18 - protein, 20 aa, Dermatophagoides pteronyssinus
  MISC_FEATURE (4)..(4): Xaa = any amino acid
AIGXQPAAEAEAPFQISLMK

SEQ ID NO: 19 - protein, 215 aa, Dermatophagoides pteronyssinus
MMKLLLIAAAAFVAVSADPIHYDKITEEINKAVDEAVAAIEKSETFDPMKVPDHSDKFERHIGIIDLKGELDMRNIQVRGLKQMKRVGDANVKSEDGVVKAHLLVGVHDDVVSMEYDLAYKLGDLHPNTHVISDIQDFVVELSLEVSEEGNMTLTSFEVRQFANVVNHIGGLSILDPIFAVLSDVLTAIFQDTVRAEMTKVLAPAFKKELERNNQ

SEQ ID NO: 20 - protein, 18 aa, Dermatophagoides pteronyssinus
IVGGSNASPGDAVYQIAL

SEQ ID NO: 21 - protein, 319 aa, Dermatophagoides farinae
MKFVLAIASLLVLTVYARPASIKTFEFKKAFNKNYATVEEEEVARKNFLESLKYVEANKGAINHLSDLSLDEFKNRYLMSAEAFEQLKTQFDLNAETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQRCRRPNSQHYGISNYCQIYPPDVKQIREALTQTHTAIAVIIGIKDLRAFQHYDGRTIIQHDNGYQPNYHAVNIVGYGSTQGDDYWIVRNSWDTTWGDSGYGYFQAGNNLMMIEQYPYVVIM

SEQ ID NO: 22 - protein, 146 aa, Dermatophagoides farinae
MISKILCLSLLVAAVVADQVDVKDCANNEIKKVMVDGCHGSDPCIIHRGKPFTLEALFDANQNTKTAKIEIKASLDGLEIDVPGIDTNACHFMKCPLVKGQQYDIKYTWNVPKIAPKSENVVVTVKLIGDNGVLACAIATHGKIRD

SEQ ID NO: 23 - protein, 259 aa, Dermatophagoides farinae
MMILTIVVLLAANILATPILPSSPNATIVGGVKAQAGDCPYQISLQSSSHFCGGSILDEYWILTAAHCVNGQSAKKLSIRYNTLKHASGGEKIQVAEIYQHENYDSMTIDNDVALIKLKTPMTLDQTNAKPVPLPAQGSDVKVGDKIRVSGWGYLQEGSYSLPSELQRVDIDVVSREQCDQLYSKAGADVSENMICGGDVANGGVDSCQGDSGGPVVDVATKQIVGIVSWGYGCARKGYPGVYTRVGNFVDWIESKRSQ

SEQ ID NO: 24 - protein, 20 aa, Dermatophagoides farinae
AVGGQDADLAEAPFQISLLK

SEQ ID NO: 25 - protein, 213 aa, Dermatophagoides farinae
MMKFLLIAAVAFVAVSADPIHYDKITEEINKAIDDAIAAIEQSETIDPMKVPDHADKFERHVGIVDFKGELAMRNIEARGLKQMKRQGDANVKGEEGIVKAHLLIGVHDDIVSMEYDLAYKLGDLHPTTHVISDIQDFVVALSLEISDEGNITMTSFEVRQFANVVNHIGGLSILDPIFGVLSDVLTAIFQDTVRKEMTKVLAPAFKRELEKN

SEQ ID NO: 26 - protein, 109 aa, Felis domesticus
MRGALLVLALLVTQALGVKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 27 - protein, 88 aa, Felis domesticus
MLDAALPPCPTVAATADCEICPAVKRDVDLFLTGTPDEYVEQVAQYKALPVVLENARILKNCVDAKMTEEDKENALSLLDKIYTSPLC

SEQ ID NO: 28 - protein, 92 aa, Felis domesticus
MKGARVLVLLWAALLLIWGGNCEICPAVKRDVDLFLTGTPDEYVEQVAQYKALPVVLENARILKNCVDAKMTEEDKENALSLLDKIYTSPLC

SEQ ID NO: 29 - protein, 138 aa, Hevea brasiliensis
MAEDEDNQQGQGEGLKYLGFVQDAATYAVTTFSNVYLFAKDKSGPLQPGVDIIEGPVKNVAVPLYNRFSYIPNGALKFVDSTVVASVTIIDRSLPPIVKDASIQVVSAIRAAPEAARSLASSLPGQTKILAKVFYGEN

SEQ ID NO: 30 - protein, 204 aa, Hevea brasiliensis
MAEEVEEERLKYLDFVRAAGVYAVDSFSTLYLYAKDISGPLKPGVDTIENVVKTVVTPVYYIPLEAVKFVDKTVDVSVTSLDGVVPPVIKQVSAQTYSVAQDAPRIVLDVASSVFNTGVQEGAKALYANLEPKAEQYAVITWRALNKLPLVPQVANVVVPTAVYFSEKYNDVVRGTTEQGYRVSSYLPLLPTEKITKVFGDEAS

SEQ ID NO: 31 - protein, 263 aa, Lolium perenne
MASSSSVLLVVALFAVFLGSAHGIAKVPPGPNITAEYGDKWLDAKSTWYGKPTGAGPKDNGGACGYKNVDKAPFNGMTGCGNTPIFKDGRGCGSCFEIKCTKPESCSGEAVTVTITDDNEEPIAPYHFDLSGHAFGSMAKKGEEQNVRSAGELELQFRRVKCKYPDDTKPTFHVEKASNPNYLAILVKYVDGDGDVVAVDIKEKGKDKWIELKESWGAVWRIDTPDKLTGPFTVRYTTEGGTKSEFEDVIPEGWKADTSYSAK

SEQ ID NO: 32 - protein, 97 aa, Lolium perenne
AAPVEFTVEKGSDEKNLALSIKYNKEGDSMAEVELKEHGSNEWLALKKNGDGVWEIKSDKPLKGPFNFRFVSEKGMRNVFDDVVPADFKVGTTYKPE

SEQ ID NO: 33 - protein, 97 aa, Lolium perenne
TKVDLTVEKGSDAKTLVLNIKYTRPGDTLAEVELRQHGSEEWEPMTKKGNLWEVKSAKPLTGPMNFRFLSKGGMKNVFDEVIPTAFTVGKTYTPEYN

SEQ ID NO: 34 - protein, 308 aa, Lolium perenne
MAVQKYTVALFLRRGPRGGPGRSYAADAGYTPAAAATPATPAATPAGGWREGDDRRAEAAGGRQRLASRQPWPPLPTPLRRTSSRSSRPPSPSPPRASSPTSAAKAPGLIPKLDTAYDVAYKAAEAHPRGQVRRLRHCPHRSLRVIAGALEVHAVKPATEEVLAAKIPTGELQIVDKIDAAFKIAATAANAAPTNDKFTVFESAFNKALNECTGGAMRPTSSSPPSRPRSSRPTPPPSPAAPEVKYAVFEAALTKAITAMTQAQKAGKPAAAAATAAATVATAAATAAAVLPPPLLVVQSLISLLIYY

SEQ ID NO: 35 - protein, 339 aa, Lolium perenne
MAVQKHTVALFLAVALVAGPAASYAADAGYAPATPATPAAPATAATPATPATPATPAAVPSGKATTEEQKLIEKINAGFKAAVAAAAVVPPADKYKTFVETFGTATNKAFVEGLASGYADQSKNQLTSKLDAALKLAYEAAQGATPEAKYDAYVATLTEALRVIAGTLEVHAVKPAAEEVKVGAIPAAEVQLIDKVDAAYRTAATAANAAPANDKFTVFENTFNNAIKVSLGAAYDSYKFIPTLVAAVKQAYAAKQATAPEVKYTVSETALKKAVTAMSEAEKEATPAAAATATPTPAAATATATPAAAYATATPAAATATATPAAATATPAAAGGYKV

SEQ ID NO: 36 - protein, 339 aa, Lolium perenne; identical to SEQ ID NO: 35

SEQ ID NO: 37 - protein, 134 aa, Lolium perenne
  MISC_FEATURE (103)..(103): Xaa = any amino acid
DKGPGFVVTGRVYCDPCRAGFETNVSHNVEGATVAVDCRPFDGGESKLKAEATTDKDGWYKIEIDQDHQEEICEVVLAKSPDKSCSEIEEFRDRARVPLTSNXGIKQQGIRYANPIAFFRKEPLKECGGILQAY

SEQ ID NO: 38 - protein, 145 aa, Olea europaea
EDIPQPPVSQFHIQGQVYCDTCRAGFITELSEFIPGASLRLQCKDKENGDVTFTEVGYTRAEGLYSMLVERDHKNEFCEITLISSGRKDCNEIPTEGWAKPSLKFKLNTVNGTTRTVNPLGFFKKEALPKCAQVYNKLGMYPPNM

SEQ ID NO: 39 - protein, 133 aa, Parietaria judaica
MRTVSMAALVVIAAALAWTSSAEPAPAPAPGEEACGKVVQDIMPCLHFVKGEEKEPSKECCSGTKKLSEEVKTTEQKREACKCIVRATKGISGIKNELVAEVPKKCDIKTTLPPITADFDCSKIQSTIFRGYY

SEQ ID NO: 40 - protein, 133 aa, Parietaria judaica
MVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSDPAHKARLERPQIRVPPPAPEKA

SEQ ID NO: 41 - protein, 133 aa, Parietaria judaica
MRTVSMAALVVIAAALAWTSSAELASAPAPGEGPCGKVVHHIMPCLKFVKGEEKEPSKSCCSGTKKLSEEVKTTEQKREACKCIVAATKGISGIKNELVAEVPKKCGITTTLPPITADFDCSKIESTIFRGYY

SEQ ID NO: 42 - protein, 176 aa, Parietaria judaica
MRTVSAPSAVALVVIVAAGLAWTSLASVAPPAPAPGSEETCGTVVRALMPCLPFVQGKEKEPSKGCCSGAKRLDGETKTGLQRVHACECIQTAMKTYSDIDGKLVSEVPKHCGIVDSKLPPIDVNMDCKTLGVVPRQPQLPVSLRHGPVTGPSDPAHKARLERPQIRVPPPAPEKA

SEQ ID NO: 43 - protein, 138 aa, Parietaria judaica
MRTVSARSSVALVVIVAAVLVWTSSASVAPAPAPGSEETCGTVVGALMPCLPFVQGKEKEPSKGCCSGAKRLDGETKTGPQRVHACECIQTAMKTYSDIDGKLVSEVPKHCGIVDSKLPPIDVNMDCKTLGVLHYKGN

SEQ ID NO: 44 - protein, 143 aa, Parietaria judaica
MVRALMPCLPFVQGKEKEPSKGCCSGAKRLDGETKTGPQRVHACECIQTAMKTYSDIDGKLVSEVPKHCGIVDSKLPPIDVNMDCKTVGVVPRQPQLPVSLRHGPVTGPSRSRPPTKHGWRDPRLEFRPPHRKKPNPAFSTLG

SEQ ID NO: 45 - protein, 263 aa, Phleum pratense
MASSSSVLLVVVLFAVFLGSAYGIPKVPPGPNITATYGDKWLDAKSTWYGKPTGAGPKDNGGACGYKDVDKPPFSGMTGCGNTPIFKSGRGCGSCFEIKCTKPEACSGEPVVVHITDDNEEPIAPYHFDLSGHAFGAMAKKGDEQKLRSAGELELQFRRVKCKYPEGTKVTFHVEKGSNPNYLALLVKYVNGDGDVVAVDIKEKGKDKWIELKESWGAIWRIDTPDKLTGPFTVRYTTEGGTKTEAEDVIPEGWKADTSYESK

SEQ ID NO: 46 - protein, 262 aa, Phleum pratense
MASSSSVLLVVALFAVFLGSAHGIPKVPPGPNITATYGDKWLDAKSTWYGKPTAAGPKDNGGACGYKDVDKPPFSGMTGCGNTPIFKSGRGCGSCFEIKCTKPEACSGEPVVVHITDDNEEPIAAYHFDLSGIAFGSMAKKGDEQKLRSAGEVEIQFRRVKCKYPEGTKVTFHVEKGSNPNYLALLVKFSGDGDVVAVDIKEKGKDKWIALKESWGAIWRIDTPEVLKGPFTVRYTTEGGTKARAKDVIPEGWKADTAYESK

SEQ ID NO: 47 - protein, 122 aa, Phleum pratense
MSMASSSSSSLLAMAVLAALFAGAWCVPKVTFTVEKGSNEKHLAVLVKYEGDTMAEVELREHGSDEWVAMTKGEGGVWTFDSEEPLQGPFNFRFLTEKGMKNVFDDVVPEKYTIGATYAPEE

SEQ ID NO: 48 - protein, 276 aa, Phleum pratense
ADLGYGGPATPAAPAEAAPAGKATTEEQKLIEKINDGFKAALAAAAGVPPADKYKTFVATFGAASNKAFAEGLSAEPKGAAESSSKAALTSKLDAAYKLAYKTAEGATPEAKYDAYVATLSEALRIIAGTLEVHAVKPAAEEVKVIPAGELQVIEKVDSAFKVAATAANAAPANDKFTVFEAAFNNAIKASTGGAYESYKFIPALEAAVKQAYAATVATAPEVKYTVFETALKKAFTAMSEAQKAAKPATEATATATAAVGAATGAATAATGGYKV

SEQ ID NO: 49 - protein, 276 aa, Phleum pratense
ADLGYGGPATPAAPAEAAPAGKATTEEQKLIEKINDGFKAALAAAAGVPPADKYKTFVATFGAASNKAFAEGLSAEPKGAAESSSKAALTSKLDAAYKLAYKTAEGATPEAKYDAYVATLSEALRIIAGTLEVHAVKPAAEEVKVIPAGELQVIEKVDSAFKVAATAANAAPANDKFTVFEAAFNNAIKASTGGAYESYKFIPALEAAVKQAYAATVATAPEVKYTVFETALKKAITAMSEAQKAAKPATEATATATAAVGAATGAATAATGGYKV

SEQ ID NO: 50 - protein, 284 aa, Phleum pratense
AAAAVPRRGPRGGPGRSYTADAGYAPATPAAAGAAAGKATTEEQKLIEDINVGFKAAVAAAASVPAADKFKTFEAAFTSSSKAAAAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 51 - protein, 286 aa, Phleum pratense
ADLGYGPATPAAPAAGYTPATPAAPAGADAAGKATTEEQKLIEKINAGFKAALAGAGVQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDEIKASTGGAYESYKFIPALEAAVKQAYAATVATAPEVKYTVFETALKKAITAMSEAQKAAKPAAAATATATAAVGAATGAATAATGGYKV

SEQ ID NO: 52 - protein, 287 aa, Phleum pratense
MAVQKYTVALFLAVALVAGPAASYAADAGYAPATPAAAGAEAGKATTEEQKLIEDINVGFKAAVAAAASVPAADKFKTFEAAFTSSSKAATAKAPGLVPKLDAAYSVSYKAAVGATPEAKFDSFVASLTEALRVIAGALEVHAVKPVTEEPGMAKIPAGELQIIDKIDAAFKVAATAAATAPADTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 53 - protein, 290 aa, Phleum pratense
MAVQKYTVALFLAVALVAGPAASYAADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEDPAWPKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTATGAASGAATVAAGGYKV

SEQ ID NO: 54 - protein, 265 aa, Phleum pratense
ADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 55 - protein, 295 aa, Phleum pratense
SVKRSNGSAEVHRGAVPRRGPRGGPGRSYAADAGYAPATPAAAGAEAGKATTEEQKLIEDINVGFKAAVAAAASVPAADKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 56 - protein, 312 aa, Phleum pratense
MAVHQYTVALFLAVALVAGPAGSYAADLGYGPATPAAPAAGYTPATPAAPAGAEPAGKATTEEQKLIEKINAGFKAALAAAAGVPPADKYRTFVATFGAASNKAFAEGLSGEPKGAAESSSKAALTSKLDAAYKLAYKTAEGATPEAKYDAYVATVSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 57 - protein, 276 aa, Phleum pratense; identical to SEQ ID NO: 48

SEQ ID NO: 58 - protein, 284 aa, Phleum pratense; identical to SEQ ID NO: 50

SEQ ID NO: 59 - protein, 286 aa, Phleum pratense; identical to SEQ ID NO: 51

SEQ ID NO: 60 - protein, 281 aa, Phleum pratense
AVPRRGPRGGPGRSYAADAGYAPATPAAAGAEAGKATTEEQKLIEDINVGFKAAVAAAASVPAGDKFKTFEAAFTSSSKAATAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTATGAASGAATVAAGGYKV

SEQ ID NO: 61 - protein, 280 aa, Phleum pratense
MAVPRRGPRGGPGRSYTADAGYAPATPAAAGAAAGKATTEEQKLIEDINVGFKAAVAARQRPAADKFKTFEAASPRHPRPLRQGAGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAAEVKYAVFEAALTKAITAMSEVQKVSQPATGAATVAAGAATTAAGAASGAATVAAGGYKV

SEQ ID NO: 62 - protein, 312 aa, Phleum pratense
MAVHQYTVALFLAVALVAGPAASYAADLGYGPATPAAPAAGYTPATPAAPAEAAPAGKATTEEQKLIEKINAGFKAALAAAAGVQPADKYRTFVATFGAASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 63 - protein, 257 aa, Phleum pratense
EAPAGKATTEEQKLIEKINAGFKAALARRLQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAAELQVIEKVDAAFKVAATAANAAPANDKFTVFEAAFNDEIKASTGGAYESYKFIPALEAAVKQAYAATVATAPEVKYTVFETALKKAITAMSEAQKAAKPPPLPPPPQPPPLAATGAATAATGGYKV

SEQ ID NO: 64 - protein, 312 aa, Phleum pratense; identical to SEQ ID NO: 62

SEQ ID NO: 65 - protein, 280 aa, Phleum pratense; identical to SEQ ID NO: 61

SEQ ID NO: 66 - protein, 285 aa, Phleum pratense
ADLGYGPATPAAPAAGYTPATPAAPAGADAAGKATTEEQKLIEKINAGFKAALAGAGVQPADKYRTFVATFGPASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDEIKASTGGAYESYKFIPALEAAVKQAYAATVATAPEVKYTVFETALKKAITAMSEAQKAAKPPPLPPPPQPPPLAATGAATAATGGYKV

SEQ ID NO: 67 - protein, 312 aa, Phleum pratense; identical to SEQ ID NO: 62

SEQ ID NO: 68 - protein, 138 aa, Phleum pratense
MAAHKFMVAMFLAVAVVLGLATSPTAEGGKATTEEQKLIEDVNASFRAAMATTANVPPADKYKTFEAAFTVSSKRNLADAVSKAPQLVPKLDEVYNAAYNAADHAAPEDKYEAFVLHFSEALRIIAGTPEVHAVKPGA

SEQ ID NO: 69 - protein, 57 aa, Phleum pratense
SKAPQLVPKLDEVYNAAYNAADHAAPEDKYEAFVLHFSEALHIIAGTPEVHAVKPGA

SEQ ID NO: 70 - protein, 80 aa, Phleum pratense
ADKYKTFEAAFTVSSKRNLADAVSKAPQLVPKLDEVYNAAYNAADHAAPEDKYEAFVLHFSEALHIIAGTPEVHAVKPGA

SEQ ID NO: 71 - protein, 106 aa, Phleum pratense
TEEQKLIEDVNASFRAAMATTANVPPADKYKTLEAAFTVSSKRNLADAVSKAPQLVPKLDEVYNAAYNAADHAAPEDKYEAFVLHFSEALRIIAGTPEVHAVKPGA

SEQ ID NO: 72 - protein, 138 aa, Phleum pratense
MAAHKFMVAMFLAVAVVLGLATSPTAEGGKATTEEQKLIEDINASFRAAMATTANVPPADKYKTFEAAFTVSSKRNLADAVSKAPQLVPKLDEVYNAAYNAADHAAPEDKYEAFVLHFSEALHIIAGTPEVHAVKPGA

SEQ ID NO: 73 - protein, 132 aa, Phleum pratense
MVAMFLAVAVVLGLATSPTAEGGKATTEEQKLIEDVNASFRAAMATTANVPPADKYKTFEAAFTVSSKRNLADAVSKAPQLVPKLDEVYNAAYNAADHAAPEDKYEAFVLHFSEALRIIAGTPEVHAVKPGA

SEQ ID NO: 74 - protein, 78 aa, Phleum pratense
MADDMERIFKRFDTNGDGKISLSELTDALRTLGSTSADEVQRMMAEIDTDGDGFIDFNEFISFCNANPGLMKDVAKVF

SEQ ID NO: 75 - protein, 131 aa, Phleum pratense
MSWQTYVDEHLMCEIEGHHLASAAILGHDGTVWAQSADFPQFKPEEITGIMKDFDEPGHLAPTGMFVAGAKYMVIQGEPGRVIRGKKGAGGITIKKTGQALVVGIYDEPMTPGQCNMVVERLGDYLVEQGM

SEQ ID NO: 76 - protein, 227 aa, Vespula vulgaris
MEISGLVYLIIIVTIIDLPYGKANNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFMNEELYQTK

SEQ ID NO: 77 - protein, 300 aa, Vespula vulgaris
GPKCPFNSDTVSIIIETRENRNRDLYTLQTLQNHPEFKKKTITRPVVFITHGFTSSASEKNFINLAKALVDKDNYMVISIDWQTAACTNEYPGLKYAYYPTAASNTRLVGQYIATITQKLVKDYKISMANIRLIGHSLGAHVSGFAGKRVQELKLGKYSEIIGLDPARPSFDSNHCSERLCETDAEYVQIIHTSNYLGTEKILGTVDFYMNNGKNNPGCGRFFSEVCSHTRAVIYMAECIKHECCLIGIPRSKSSQPISRCTKQECVCVGLNAKKYPSRGSFYVPVESTAPFCNNKGKII

SEQ ID NO: 78 - protein, 336 aa, Vespula vulgaris
MEENMNLKYLLLFVYFVQVLNCCYGHGDPLSYELDRGPKCPFNSDTVSIIIETRENRNRDLYTLQTLQNHPEFKKKTITRPVVFITHGFTSSASETNFINLAKALVDKDNYMVISIDWQTAACTNEAAGLKYLYYPTAARNTRLVGQYIATITQKLVKHYKISMANIRLIGHSLGAHASGFAGKKVQELKLGKYSEIIGLDPARPSFDSNHCSERLCETDAEYVQIIHTSNYLGTEKTLGTVDFYMNNGKNQPGCGRFFSEVCSHSRAVIYMAECIKHECCLIGIPKSKSSQPISSCTKQECVCVGLNAKKYPSRGSFYVPVESTAPFCNNKGKII

SEQ ID NO: 79 - protein, 331 aa, Vespula vulgaris
SERPKRVFNIYWNVPTFMCHQYDLYFDEVTNFNIKRNSKDDFQGDKIAIFYDPGEFPALLSLKDGKYKKRNGGVPQEGNITIHLQKFIENLDKIYPNRNFSGIGVIDFERWRPIFRQNWGNMKIHKNFSIDLVRNEHPTWNKKMIELEASKRFEKYARFFMEETLKLAKKTRKQADWGYYGYPYCFNMSPNNLVPECDVTAMHENDKMSWLFNNQNVLLPSVYVRQELTPDQRIGLVQGRVKEAVRISNNLKHSPKVLSYWWYVYQDETNTFLTETDVKKTFQEIVINGGDGIIIWGSSSDVNSLSKCKRLQDYLLTVLGPIAINVTEAVN

SEQ ID NO: 80 - protein, 206 aa, Vespula vulgaris
KVNYCKIKCLKGGVHTACKYGTSTKPNCGKMVVKAYGLTEAEKQEILKVHNDFRQKVAKGLETRGNPGPQPPAKNMNNLVWNDELANIAQVWASQCNYGHDTCKDTEKYPVGQNIAKRSTTAALFDSPGKLVKMWENEVKDFNPNIEWSKNNLKKTGHYTQMVWAKTKEIGCGSVKYVKDEWYTHYLVCNYGPSGNFRNEKLYEKK

SEQ ID NO: 81 - protein, 160 aa, Betula pendula
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 82 - protein, 133 aa, Betula pendula
MSWQTYVDEHLMCDIDGQASNSLASAIVGHDGSVWAQSSSFPQFKPQEITGIMKDFEEPGHLAPTGLHLGGIKYMVIQGEAGAVIRGKKGSGGITIKKTGQALVFGIYEEPVTPGQCNMVVERLGDYLIDQGL

SEQ ID NO: 83 - protein, 205 aa, Betula pendula
MPCSTEAMEKAGHGHASTPRKRSLSNSSFRLRSESLNTLRLRRIFDLFDKNSDGIITVDELSRALNLLGLETDLSELESTVKSFTREGNIGLQFEDFISLHQSLNDSYFAYGGEDEDDNEEDMRKSILSQEEADSFGGFKVFDEDGDGYISARELQMVLGKLGFSEGSEIDRVEKMIVSVDSNRDGRVDFFEFKDMMRSVLVRSS

SEQ ID NO: 84 - protein, 85 aa, Betula pendula
MADDHPQDKAERERIFKRFDANGDGKISAAELGEALKTLGSITPDEVKHMMAEIDTDGDGFISFQEFTDFGRANRGLLKDVAKIF

SEQ ID NO: 85 - protein, 24 aa, Quercus alba
  MISC_FEATURE (5)..(17): Xaa = any amino acid
GVFTXESQETSVIAPAXLFKALFL

SEQ ID NO: 86 - protein, 40 aa, Carpinus betulus
  MISC_FEATURE (39)..(39): Xaa = any amino acid
GVFNYEAETPSVIPAARLFKSYVLDGDKLIPKVAPQAIXK

SEQ ID NO: 87 - protein, 44 aa, Alnus glutinosa
GVFNYEAETPSVIPAARLFKAFILDGDKLLPKVAPEAVSSVENI

SEQ ID NO: 88 - protein, 110 aa, Betual pendula
VQCMQVWPPLGLKKFETLSYLPPLSSEQLAKEVDYLLRKNLIPCLEFELEHGFVYREHNRSPGYYDGRYWTMWKLPMFGCNDSSQVLKELEECKKAYPSAFIRIIGFDDK

SEQ ID NO: 89 - protein, 626 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 90 - protein, 392 aa, Ambrosia artemisiifolia
MGIKHCCYILYFTLALVTLLQPVRSAEDLQQILPSANETRSLTTCGTYNIIDGCWRGKADWAENRKALADCAQGFAKGTIGGKDGDIYTVTSELDDDVANPKEGTLRFGAAQNRPLWIIFARDMVIRLDRELAINNDKTIDGRGAKVEIINAGFAIYNVKNIIIHNIIMHDIVVNPGGLIKSHDGPPVPRKGSDGDAIGISGGSQIWIDHCSLSKAVDGLIDAKHGSTHFTVSNCLFTQHQYLLLFWDFDERGMLCTVAFNKFTDNVDQRMPNLRHGFVQVVNNNYERWGSYALGGSAGPTILSQGNRFLASDIKKEVVGRYGESAMSESINWNWRSYMDVFENGAIFVPSGVDPVLTPEQNAGMIPAEPGEAVLRLTSSAGVLSCQPGAPC

SEQ ID NO: 91 - protein, 397 aa, Ambrosia artemisiifolia
MGIKHCCYILYFTLALVTLVQAGRLGEEVDILPSPNDTRRSLQGCEAHNIIDKCWRCKPDWAENRQALGNCAQGFGKATHGGKWGDIYMVTSDQDDDVVNPKEGTLRFGATQDRPLWIIFQRDMIIYLQQEMVVTSDKTIDGRGAKVELVYGGITLMNVKNVIIHNIDIHDVRVLPGGRIKSNGGPAIPRHQSDGDAIHVTGSSDIWIDHCTLSKSFDGLVDVNWGSTGVTISNCKFTHHEKAVLLGASDTHFQDLKMHVTLAYNIFTNTVHERMPRCRFGFFQIVNNFYDRWDKYAIGGSSNPTILSQGNKFVAPDFIYKKNVCLRTGAQEPEWMTWNWRTQNDVLENGAIFVASGSDPVLTAEQNAGMMQAEPGDMVPQLTMNAGVLTCSPGAPC

SEQ ID NO: 92 - protein, 397 aa, Ambrosia artemisiifolia
MGIKQCCYILYFTLALVALLQPVRSAEGVGEILPSVNETRSLQACEALNIIDKCWRGKADWENNRQALADCAQGFAKGTYGGKWGDVYTVTSNLDDDVANPKEGTLRFAAAQNRPLWIIFKNDMVINLNQELVVNSDKTIDGRGVKVEIINGGLTLMNVKNIIIHNINIHDVKVLPGGMIKSNDGPPILRQASDGDTINVAGSSQIWIDHCSLSKSFDGLVDVTLGSTHVTISNCKFTQQSKAILLGADDTHVQDKGMLATVAFNMFTDNVDQRMPRCRFGFFQVVNNNYDRWGTYAIGGSSAPTILCQGNRFLAPDDQIKKNVLARTGTGAAESMAWNWRSDKDLLENGAIFVTSGSDPVLTPVQSAGMIPAEPGEAAIKLTSSAGVFSCHPGAPC

SEQ ID NO: 93 - protein, 398 aa, Ambrosia artemisiifolia
MGIKHCCYILYFTLALVTLLQPVRSAEDVEEFLPSANETRRSLKACEAHNIIDKCWRCKADWANNRQALADCAQGFAKGTYGGKHGDVYTVTSDKDDDVANPKEGTLRFAAAQNRPLWIIFKRNMVIHLNQELVVNSDKTIDGRGVKVNIVNAGLTLMNVKNIIIHNINIHDIKVCPGGMIKSNDGPPILRQQSDGDAINVAGSSQIWIDHCSLSKASDGLLDITLGSSHVTVSNCKFTQHQFVLLLGADDTHYQDKGMLATVAFNMFTDHVDQRMPRCRFGFFQVVNNNYDRWGTYAIGGSSAPTILSQGNRFFAPDDIIKKNVLARTGTGNAESMSWNWRTDRDLLENGAIFLPSGSDPVLTPEQKAGMIPAEPGEAVLRLTSSAGVLSCHQGAPC

SEQ ID NO: 94 - protein, 396 aa, Ambrosia artemisiifolia
MGIKHCCYILYFTLALVTLLQPVRSAEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 95 - protein, 373 aa, Cryptomeria japonica
MDSPCLVALLVFSFVIGSCFSDNPIDSCWRGDSNWAQNRMKLADCAVGFGSSTMGGKGGDLYTVTNSDDDPVNPPGTLRYGATRDRPLWIIFSGNMNIKLKMPMYIAGYKTFDGRGAQVYIGNGGPCVFIKRVSNVIIHGLYLYGCSTSVLGNVLINESFGVEPVHPQDGDALTLRTATNIWIDHNSFSNSSDGLVDVTLTSTGVTISNNLFFNHHKVMSLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPHLTQNAGVLTCSLSKRC

SEQ ID NO: 96 - protein, 374 aa, Cryptomeria japonica
MDSPCLVALLVLSFVIGSCFSDNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRC

SEQ ID NO: 97 - protein, 514 aa, Cryptomeria japonica
MAMKLIAPMAFLAMQLIIMAAAEDQSAQIMLDSVVEKYLRSNRSLRKVEHSRHDAINIFNVEKYGAVGDGKHDCTEAFSTAWQAACKNPSAMLLVPGSKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKDIKLSDISLKLTSGKIASCLNDNANGYFSGHVIPACKNLSPSAKRKESKSHKHPKTVMVENMRAYDKGNRTRILLGSRPPNCTNKCHGCSPCKAKLVIVHRIMPQEYYPQRWICSCHGKIYHP

SEQ ID NO: 98 - protein, 514 aa, Cryptomeria japonica
MAMKFIAPMAFVAMQLIIMAAAEDQSAQIMLDSDIEQYLRSNRSLRKVEHSRHDAINIFNVEKYGAVGDGKHDCTEAFSTAWQAACKKPSAMLLVPGNKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKDIKLSDISLKLTSGKIASCLNDNANGYFSGHVIPACKNLSPSAKRKESKSHKHPKTVMVKNMGAYDKGNRTRILLGSRPPNCTNKCHGCSPCKAKLVIVHRIMPQEYYPQRWMCSRHGKIYHP

SEQ ID NO: 99 - protein, 373 aa, Cryptomeria japonica
MDSPCLVALLVLSFVIGSCFSDNPIDSCWRGDSNWAQNRMKLADCAVGFGSSTMGGKGGDLYTVTNSDDDPVNPPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRC

SEQ ID NO: 100 - protein, 374 aa, Cryptomeria japonica
MDSPCLVALLVFSFVIGSCFSDNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLYLYGCSTSVLGNVLINESFGVEPVHPQDGDALTLRTATNIWIDHNSFSNSSDGLVDVTLTSTGVTISNNLFFNHHKVMSLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPHLTQNAGVLTCSLSKRC

SEQ ID NO: 101 - protein, 174 aa, Canis familiaris
MKTLLLTIGFSLIAILQAQDTPALGKDTVAVSGKWYLKAMTADQEVPEKPDSVTPMILKAQKGGNLEAKITMLTNGQCQNITVVLHKTSEPGKYTAYEGQRVVFIQPSPVRDHYILYCEGELHGRQIRMAKLLGRDPEQSQEALEDFREFSRAKGLNQEILELAQSETCSPGGQ

SEQ ID NO: 102 - protein, 24 aa, Canis familiaris
EAYKSEIAHRYNDLGEEHFRGLVL

SEQ ID NO: 103 - protein, 265 aa, Canis familiaris
LSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVVEVSRKLGKVGTKCCKKPESERMSCADDFLS

SEQ ID NO: 104 - protein, 180 aa, Canis familiaris
MQLLLLTVGLALICGLQAQEGNHEEPQGGLEELSGRWHSVALASNKSDLIKPWGHFRVFIHSMSAKDGNLHGDILIPQDGQCEKVSLTAFKTATSNKFDLEYWGHNDLYLAEVDPKSYLILYMINQYNDDTSLVAHLMVRDLSRQQDFLPAFESVCEDIGLHKDQIVVLSDDDRCQGSRD

SEQ ID NO: 105 - protein, 187 aa, Equus caballus
MKLLLLCLGLILVCAQQEENSDVAIRNFDISKISGEWYSIFLASDVKEKIEENGSMRVFVDVIRALDNSSLYAEYQTKVNGECTEFPMVFDKTEEDGVYSLNYDGYNVFRISEFENDEHIILYLVNFDKDRPFQLFEFYAREPDVSPEIKEEFVKIVQKRGIVKENIIDLTKIDRCFQLRGNGVAQA

SEQ ID NO: 106 - protein, 29 aa, Equus caballus
  MISC_FEATURE (2)..(28): Xaa = any amino acid
SQXPQSETDYSQLSGEWNTIYGAASNIXK

SEQ ID NO: 107 - protein, 211 aa, Euroglyphus maynei
TYACSINSVSLPSELDLRSLRTVTPIRMQGGCGSCWAFSGVASTESAYLAYRNMSLDLAEQELVDCASQNGCHGDTIPRGIEYIQQNGVVQEHYYPYVAREQSCHRPNAQRYGLKNYCQISPPDSNKIRQALTQTHTAVAVIIGIKDLNAFRHYDGRTIMQHDNGYQPNYHAVNIVGYGNTQGVDYWIVRNSWDTTWGDNGYGYFAANINL

SEQ ID NO: 108 - protein, 211 aa, Euroglyphus maynei; identical to SEQ ID NO: 107

SEQ ID NO: 109 - protein, 211 aa, Euroglyphus maynei
ETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDL

SEQ ID NO: 110 - protein, 212 aa, Euroglyphus maynei
ETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQQCRRPNSQHYGISNYCQIYPPDVKQIREALTQTHTAIAVIIGIKDLRAFQHYDGRTIIQHDNGYQPNYHAVNIVGYGSTQGVDYWIVRNSWDTTWGDSGYGYFQAGNNL

SEQ ID NO: 111 - protein, 307 aa, Poa pratensis
MAVQKYTVALFLVALVVGPAASYAADLSYGAPATPAAPAAGYTPAAPAGAAPKATTDEQKMIEKINVGFKAAVAAAGGVPAANKYKTFVATFGAASNKAFAEALSTEPKGAAVDSSKAALTSKLDAAYKLAYKSAEGATPEAKYDDYVATLSEALRIIAGTLEVHGVKPAAEEVKATPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAATGTATAAVGAATGAATAAAGGYKV

SEQ ID NO: 112 - protein, 333 aa, Poa pratensis
MAVHQYTVALFLAVALVAGPAASYAADVGYGAPATLATPATPAAPAAGYTPAAPAGAAPKATTDEQKLIEKINAGFKAAVAAAAGVPAVDKYKTFVATFGTASNKAFAEALSTEPKGAAAASSNAVLTSKLDAAYKLAYKSAEGATPEAKYDAYVATLSEALRIIAGTLEVHAVKPAGEEVKAIPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAVTATATGAVGAATGAVGAATGAATAAAGGYKTGAATPTAGGYKV

SEQ ID NO: 113 - protein, 373 aa, Poa pratensis
MDKANGAYKTALKAASAVAPAEKFPVFQATFDKNLKEGLSGPDAVGFAKKLDAFIQTSYLSTKAAEPKEKFDLFVLSLTEVLRFMAGAVKAPPASKFPAKPAPKVAAYTPAAPAGAAPKATTDEQKLIEKINVGFKAAVAAAAGVPAASKYKTFVATFGAASNKAFAEALSTEPKGAAVASSKAVLTSKLDAAYKLAYKSAEGATPEAKYDAYVATLSEALRIIAGTLEVHGVKPAAEEVKAIPAGELQVIDKVDAAFKVAATAANAAPANDKFTVFEAAFNDAIKASTGGAYQSYKFIPALEAAVKQSYAATVATAPAVKYTVFETALKKAITAMSQAQKAAKPAAAVTGTATSAVGAATGAATAAAGGYKV

SEQ ID NO: 114 - protein, 685 aa, Periplaneta americana
MKTALVFAAVVAFVAARFPDHKDYKQLADKQFLAKQRDVLRLFHRVHQHNILNDQVEVGIPMTSKQTSATTVPPSGEAVHGVLQEGHARPRGEPFSVNYEKHREQAIMLYDLLYFANDYDTFYKTACWARDRVNEGMFMYSFSIAVFHRDDMQGVMLPPPYEVYPYLFVDHDVIHMAQKYWMKNAGSGEHHSHVIPVNFTLRTQDHLLAYFTSDVNLNAFNTYYRYYYPSWYNTTLYGHNIDRRGEQFYYTYKQIYARYFLERLSNDLPDVYPFYYSKPVKSAYNPNLRYHNGEEMPVRPSNMYVTNFDLYYIADIKNYEKRVEDAIDFGYAFDEHMKPHSLYHDVHGMEYLADMIEGNMDSPNFYFYGSIYHMYHSMIGHIVDPYHKMGLAPSLEHPETVLRDPVFYQLWKRVDHLFQKYKNRLPRYTHDELAFEGVKVENVDVGKLYTYFEQYDMSLDMAVYVNNVDQISNVDVQLAVRLNHKPFTYNIEVSSDKAQDVYVAVFLGPKYDYLGREYDLNDRRHYFVEMDRFPYHVGAGKTVIERNSHDSNIIAPERDSYRTFYKKVQEAYEGKSQYYVDKGHNYCGYPENLLIPKGKKGGQAYTFYVIVTPYVKQDEHDFEPYNYKAFSYCGVGSERKYPDNKPLGYPFDRKIYSNDFYTPNMYFKDVIIFHKKYDEVGVQGH

SEQ ID NO: 115 - protein, 446 aa, Periplaneta americana
INEIHSIIGLPPFVPPSRRHARRGVGINGLIDDVIAILPVDELKALFQEKLETSPDFKALYDAIRSPEFQSIISTLNAMQRSEHHQNLRDKGVDVDHFIQLIRALFGLSRAARNLQDDLNDFLHSLEPISPRHRHGLPRQRRRSARVSAYLHADDFHKIITTIEALPEFANFYNFLKEHGLDVVDYINEIHSIIGLPPFVPPSRRHARRGVGINGLIDDVIAILPVDELKALFQEKLETSPDFKALYDAIRSPEFQSIISTLNAMPEYQELLQNLRDKGVDVDHFIRVDQGTLRTLSSGQRNLQDDLNDFLALIPTDQILAIAMDYLANDAEVQELVAYLQSDDFHKIITTIEALPEFANFYNFLKEHGLDVVDYINEIHSIIGLPPFVPPSQRHARRGVGINGLIDDVIAILPVDELKALFQEKLETSPDFKALYDAIDLRSSRA

SEQ ID NO: 116 - protein, 352 aa, Blattella germanica
MIGLKLVTVLFAVATITHAAELQRVPLYKLVHVFINTQYAGITKIGNQNFLTVFDSTSCNVVVASQECVGGACVCPNLQKYEKLKPKYISDGNVQVKFFDTGSAVGRGIEDSLTISNLTTSQQDIVLADELSQEVCILSADVVVGIAAPGCPNALKGKTVLENFVEENLIAPVFSIHHARFQDGEHFGEIIFGGSDWKYVDGEFTYVPLVGDDSWKFRLDGVKIGDTTVAPAGTQAIIDTSKAIIVGPKAYVNPINEAIGCVVEKTTTRRICKLDCSKIPSLPDVTFVINGRNFNISSQYYIQQNGNLCYSGFQPCGHSDHFFIGDFFVDHYYSEFNWENKTMGFGRSVESV

SEQ ID NO: 117 - protein, 182 aa, Blattella germanica
AVLALCATDTLANEDCFRHESLVPNLDYERFRGSWIIAAGTSEALTQYKCWIDRFSYDDALVSKYTDSQGKNRTTIRGRTKFEGNKFTIDYNDKGKAFSAPYSVLATDYENYAIVEGCPAAANGHVIYVQIRFSVRRFHPKLGDKEMIQHYTLDQVNQHKKAIEEDLKHFNLKYEDLHSTCH

SEQ ID NO: 118 - protein, 200 aa, Blattella germanica
YKLTYCPVKALGEPIRFLLSYGEKDFEDYRFQEGDWPNLKPSMPFGKTPVLEIDGKQTHQSVAISRYLGKQFGLSGKDDWENLEIDMIVDTISDFRAAIANYHYDADENSKQKKWDPLKKETIPYYTKKFDEVVKANGGYLAAGKLTWADFYFVAILDYLNHMAKEDLVANQPNLKALREKVLGLPAIKAWVAKRPPTDL